SEQUENCE LISTING (34 sequences)

SEQ ID NO: 1 - DNA, 918 bp, Homo sapiens
  NOTCH1 Nucleic acid sequence encoding amino acids 1427-1732
cacatcctggactacagcttcgggggtggggccgggcgcgacatccccccgccgctgatcgaggaggcgtgcgagctgcccgagtgccaggaggacgcgggcaacaaggtctgcagcctgcagtgcaacaaccacgcgtgcggctgggacggcggtgactgctccctcaacttcaatgacccctggaagaactgcacgcagtctctgcagtgctggaagtacttcagtgacggccactgtgacagccagtgcaactcagccggctgcctcttcgacggctttgactgccagcgtgcggaaggccagtgcaaccccctgtacgaccagtactgcaaggaccacttcagcgacgggcactgcgaccagggctgcaacagcgcggagtgcgagtgggacgggctggactgtgcggagcatgtacccgagaggctggcggccggcacgctggtggtggtggtgctgatgccgccggagcagctgcgcaacagctccttccacttcctgcgggagctcagccgcgtgctgcacaccaacgtggtcttcaagcgtgacgcacacggccagcagatgatcttcccctactacggccgcgaggaggagctgcgcaagcaccccatcaagcgtgccgccgagggctgggccgcacctgacgccctgctgggccaggtgaaggcctcgctgctccctggtggcagcgagggtgggcggcggcggagggagctggaccccatggacgtccgcggctccatcgtctacctggagattgacaaccggcagtgtgtgcaggcctcctcgcagtgcttccagagtgccaccgacgtggccgcattcctgggagcgctcgcctcgctgggcagcctcaacatcccctacaagatcgaggccgtgcagagtgagaccgtggagccgcccccgccg

SEQ ID NO: 2 - protein, 306 aa, Homo sapiens
  NOTCH1 amino acids 1427-1732
HILDYSFGGGAGRDIPPPLIEEACELPECQEDAGNKVCSLQCNNHACGWDGGDCSLNFNDPWKNCTQSLQCWKYFSDGHCDSQCNSAGCLFDGFDCQRAEGQCNPLYDQYCKDHFSDGHCDQGCNSAECEWDGLDCAEHVPERLAAGTLVVVVLMPPEQLRNSSFHFLRELSRVLHTNVVFKRDAHGQQMIFPYYGREEELRKHPIKRAAEGWAAPDALLGQVKASLLPGGSEGGRRRRELDPMDVRGSIVYLEIDNRQCVQASSQCFQSATDVAAFLGALASLGSLNIPYKIEAVQSETVEPPPP

SEQ ID NO: 3 - DNA, 705 bp, Artificial Sequence
  52M51 Light chain polynucleotide sequence
atggcctggatttcacttatactctctctcctggctctcagctcaggggccatttcccaggctgttgtgactcaggaatctgcactcaccacatcacctggtgaaacagtcacactcacttgtcgctcaagtactggggctgttacaactagtaactacgccaactgggtccaagaaaaacctgatcatttattcactggtctaataggtggtaccaacaaccgagctccaggtgttcctgccagattctcaggctccctgattggagacaaggctgccctcaccatcacaggggcacagactgaggatgaggcaatatatttctgtgctctatggtacagcaaccactgggtgttcggtggaggaaccaaactgactgtcctaggccagcccaagtcttcgccatcagtcaccctgtttccaccttcctctgaagagctcgagactaacaaggccacactggtgtgtacgatcactgatttctacccaggtgtggtgacagtggactggaaggtagatggtacccctgtcactcagggtatggagacaacccagccttccaaacagagcaacaacaagtacatggctagcagctacctgaccctgacagcaagagcatgggaaaggcatagcagttacagctgccaggtcactcatgaaggtcacactgtggagaagagtttgtcccgtgctgactgttcctag

SEQ ID NO: 4 - protein, 234 aa, Artificial Sequence
  52M51 Light chain amino acid sequence
MAWISLILSLLALSSGAISQAVVTQESALTTSPGETVTLTCRSSTGAVTTSNYANWVQEKPDHLFTGLIGGTNNRAPGVPARFSGSLIGDKAALTITGAQTEDEAIYFCALWYSNHWVFGGGTKLTVLGQPKSSPSVTLFPPSSEELETNKATLVCTITDFYPGVVTVDWKVDGTPVTQGMETTQPSKQSNNKYMASSYLTLTARAWERHSSYSCQVTHEGHTVEKSLSRADCS

SEQ ID NO: 5 - DNA, 387 bp, Artificial Sequence
  52M51 Light chain variable region polynucleotide
atggcctggatttcacttatactctctctcctggctctcagctcaggggccatttcccaggctgttgtgactcaggaatctgcactcaccacatcacctggtgaaacagtcacactcacttgtcgctcaagtactggggctgttacaactagtaactacgccaactgggtccaagaaaaacctgatcatttattcactggtctaataggtggtaccaacaaccgagctccaggtgttcctgccagattctcaggctccctgattggagacaaggctgccctcaccatcacaggggcacagactgaggatgaggcaatatatttctgtgctctatggtacagcaaccactgggtgttcggtggaggaaccaaactgactgtcctaggc

SEQ ID NO: 6 - protein, 180 aa, Artificial Sequence
  52M51 Light chain variable region amino acid sequence
MAWISLILSLLALSSGAISQAVVTQESALTTSPGETVTLTCRSSTGAVTTSNYANWVQEKPDHLFTGLIGGTNNRAPGVPARFSGSLIGDKAALTITGAQTEDEAIYFCALWYSNHWVFGGGTKLTVLGQPKSSPSVTLFPPSSEELETNKATLVCTITDFYPGVVTVDWKVDGTPVTQG

SEQ ID NO: 7 - DNA, 330 bp, Artificial Sequence
  52M51 Light chain variable region polynucleotide sequence
caggctgttgtgactcaggaatctgcactcaccacatcacctggtgaaacagtcacactcacttgtcgctcaagtactggggctgttacaactagtaactacgccaactgggtccaagaaaaacctgatcatttattcactggtctaataggtggtaccaacaaccgagctccaggtgttcctgccagattctcaggctccctgattggagacaaggctgccctcaccatcacaggggcacagactgaggatgaggcaatatatttctgtgctctatggtacagcaaccactgggtgttcggtggaggaaccaaactgactgtcctaggc

SEQ ID NO: 8 - protein, 110 aa, Artificial Sequence
  Light chain variable region amino acid sequence
QAVVTQESALTTSPGETVTLTCRSSTGAVTTSNYANWVQEKPDHLFTGLIGGTNNRAPGVPARFSGSLIGDKAALTITGAQTEDEAIYFCALWYSNHWVFGGGTKLTVLG

SEQ ID NO: 9 - DNA, 1395 bp, Artificial Sequence
  52M51 Heavy chain polynucleotide sequence
atggaatggacctgggtctttctcttcctcctgtcagtaactgcaggtgtccactcccaggttcagctgcagcagtctggagctgagctgatgaagcctggggcctcagtgaagatatcctgcaaggctgctggctacacaatgagaggctactggatagagtggataaagcagaggcctggacatggccttgagtggattggacagattttacctggaactgggagaactaactacaatgagaagttcaagggcaaggccacattcactgcagatacatcctccaacacagccaacatgcaactcagcagcctgacatctgaggactctgccgtctattactgtgcaagatttgatggtaactacggttactatgctatggactactggggtcaaggatcctcagtcaccgtctcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagccctcggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtcctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatatccaaaaccaaaggcagaccgaaggctccacaggtgtacaccattccacctcccaaggagcagatggccaaggataaagtcagtctgacctgcatgataacagacttcttccctgaagacataacagtggagtggcagtggaatgggcagccagcggagaactacaagaacactcagcccatcatgaacacgaatggctcttacttcgtctacagcaagctcaatgtgcagaagagcaactgggaggcaggaaatactttcacctgctctgtgttacatgagggcctgcacaaccaccatactgagaagagcctctcccactctcctggtaaatga

SEQ ID NO: 10 - protein, 464 aa, Artificial Sequence
  52M51 Heavy chain amino acid sequence
MEWTWVFLFLLSVTAGVHSQVQLQQSGAELMKPGASVKISCKAAGYTMRGYWIEWIKQRPGHGLEWIGQILPGTGRTNYNEKFKGKATFTADTSSNTANMQLSSLTSEDSAVYYCARFDGNYGYYAMDYWGQGSSVTVSSAKTTPPSVYPLAPGSAAQTNSMVTLGCLVKGYFPEPVTVTWNSGSLSSGVHTFPAVLQSDLYTLSSSVTVPSSPRPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK

SEQ ID NO: 11 - DNA, 420 bp, Artificial Sequence
  52M51 Heavy chain variable region polynucleotide sequence
atggaatggacctgggtctttctcttcctcctgtcagtaactgcaggtgtccactcccaggttcagctgcagcagtctggagctgagctgatgaagcctggggcctcagtgaagatatcctgcaaggctgctggctacacaatgagaggctactggatagagtggataaagcagaggcctggacatggccttgagtggattggacagattttacctggaactgggagaactaactacaatgagaagttcaagggcaaggccacattcactgcagatacatcctccaacacagccaacatgcaactcagcagcctgacatctgaggactctgccgtctattactgtgcaagatttgatggtaactacggttactatgctatggactactggggtcaaggatcctcagtcaccgtctcctca

SEQ ID NO: 12 - protein, 180 aa, Artificial Sequence
  52M51 Heavy chain variable region amino acid sequence
MEWTWVFLFLLSVTAGVHSQVQLQQSGAELMKPGASVKISCKAAGYTMRGYWIEWIKQRPGHGLEWIGQILPGTGRTNYNEKFKGKATFTADTSSNTANMQLSSLTSEDSAVYYCARFDGNYGYYAMDYWGQGSSVTVSSAKTTPPSVYPLAPGSAAQTNSMVTLGCLVKGYFPEPVTVT

SEQ ID NO: 13 - DNA, 363 bp, Artificial Sequence
  52M51 Heavy chain variable region polynucleotide sequence
caggttcagctgcagcagtctggagctgagctgatgaagcctggggcctcagtgaagatatcctgcaaggctgctggctacacaatgagaggctactggatagagtggataaagcagaggcctggacatggccttgagtggattggacagattttacctggaactgggagaactaactacaatgagaagttcaagggcaaggccacattcactgcagatacatcctccaacacagccaacatgcaactcagcagcctgacatctgaggactctgccgtctattactgtgcaagatttgatggtaactacggttactatgctatggactactggggtcaaggatcctcagtcaccgtctcctca

SEQ ID NO: 14 - protein, 122 aa, Artificial Sequence
  52M51 Heavy chain variable region amino acid sequence
QVQLQQSGAELMKPGASVKISCKAAGYTMRGYWIEWIKQRPGHGLEWIGQILPGTGRTNYNEKFKGKATFTADTSSNTANMQLSSLTSEDSAVYYCARFDGNYGYYAMDYWGQGSSVTVSSA

SEQ ID NO: 15 - protein, 6 aa, Artificial Sequence
  52M51 Heavy chain CDR1
RGYWIE

SEQ ID NO: 16 - protein, 17 aa, Artificial Sequence
  52M51 Heavy chain CDR2
QILPGTGRTNYNEKFKG

SEQ ID NO: 17 - protein, 12 aa, Artificial Sequence
  52M51 Heavy chain CDR3
FDGNYGYYAMDY

SEQ ID NO: 18 - protein, 14 aa, Artificial Sequence
  52M51 Light chain CDR1
RSSTGAVTTSNYAN

SEQ ID NO: 19 - protein, 7 aa, Artificial Sequence
  52M51 Light chain CDR2
GTNNRAP

SEQ ID NO: 20 - protein, 16 aa, Artificial Sequence
  52M51 Light chain CDR3
ALWYSNHWVFGGGTKL

SEQ ID NO: 21 - DNA, 1401 bp, Artificial Sequence
  52M51-H4 Heavy chain polynucleotide sequence
atggattggacatggagggtgttctgcctcctcgctgtggctcctggagtcctgagccaggtccagctcgtccagagcggggctgaagtcaagaagcctggcgctagcgtcaaaatcagctgtaaggtcagcggatacacactgaggggatactggatcgagtgggtgaggcaggctccaggaaagggcctggaatggatcggccagatcctgcctggaaccggaaggacaaattacaatgagaagtttaagggaagggtcacaatgacagcagacacaagcacagacacagcttatatggaactcagctccctcagatccgaggacaccgctgtctactattgtgccaggttcgatggaaattacggatactatgccatggattactggggacaggggacaacggtcaccgtgagctcagccagcacaaagggccctagcgtcttccctctggctccctgcagcaggagcaccagcgagagcacagccgccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 22 - protein, 466 aa, Artificial Sequence
  52M51-H4 Heavy chain amino acid sequence
MDWTWRVFCLLAVAPGVLSQVQLVQSGAEVKKPGASVKISCKVSGYTLRGYWIEWVRQAPGKGLEWIGQILPGTGRTNYNEKFKGRVTMTADTSTDTAYMELSSLRSEDTAVYYCARFDGNYGYYAMDYWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 23 - protein, 141 aa, Artificial Sequence
  52M51-H4  Heavy chain variable region amino acid sequence
MDWTWRVFCLLAVAPGVLSQVQLVQSGAEVKKPGASVKISCKVSGYTLRGYWIEWVRQAPGKGLEWIGQILPGTGRTNYNEKFKGRVTMTADTSTDTAYMELSSLRSEDTAVYYCARFDGNYGYYAMDYWGQGTTVTVSSA

SEQ ID NO: 24 - protein, 122 aa, Artificial Sequence
  52M51-H4  Heavy chain variable region amino acid sequence
QVQLVQSGAEVKKPGASVKISCKVSGYTLRGYWIEWVRQAPGKGLEWIGQILPGTGRTNYNEKFKGRVTMTADTSTDTAYMELSSLRSEDTAVYYCARFDGNYGYYAMDYWGQGTTVTVSSA

SEQ ID NO: 25 - DNA, 720 bp, Artificial Sequence
  52M51-L3 Light chain polynucleotide sequence
atgagcgtccctacaatggcttggatgatgctcctgctgggactcctggcttatggaagcggagtggatagccaggccgtcgtcacacaggaacctagcctcaccgttagccctggaggaacagtcacactgacctgtaggagctccacaggagctgtgacaacaagcaattacgctaactggttccagcagaagcccggtcaagcccctagaaccctcatcggcggcaccaataacagagctcccggagtccccgccaggttctccggctccctcctgggtggcaaggctgctctgacactcagcggtgcccagccagaggatgaagcggagtactactgtgcactgtggtacagcaaccattgggttttcggaggcggaacaaagttaaccgtcctcgggcagcctaaggctgctcctagcgtcacactgttccccccatctagcgaggagctgcaggctaacaaggcaaccctcgtctgcctggttagcgacttctaccctggcgctgtcacagtggcctggaaagctgacggctcccctgtgaaagttggcgtcgaaaccacaaagccttctaagcagagcaataataaatatgccgcaagctcctacctctccctgactcctgagcagtggaaaagccataggagctactcctgccgggtcacacacgaaggaagcacagtggaaaagacagtcgcccctgctgagtgtagctga

SEQ ID NO: 26 - protein, 239 aa, Artificial Sequence
  52M51-L3 Light chain amino acid sequence
MSVPTMAWMMLLLGLLAYGSGVDSQAVVTQEPSLTVSPGGTVTLTCRSSTGAVTTSNYANWFQQKPGQAPRTLIGGTNNRAPGVPARFSGSLLGGKAALTLSGAQPEDEAEYYCALWYSNHWVFGGGTKLTVLGQPKAAPSVTLFPPSSEELQANKATLVCLVSDFYPGAVTVAWKADGSPVKVGVETTKPSKQSNNKYAASSYLSLTPEQWKSHRSYSCRVTHEGSTVEKTVAPAECS

SEQ ID NO: 27 - protein, 134 aa, Artificial Sequence
  52M51-L3 Light chain variable region amino acid sequence
MSVPTMAWMMLLLGLLAYGSGVDSQAVVTQEPSLTVSPGGTVTLTCRSSTGAVTTSNYANWFQQKPGQAPRTLIGGTNNRAPGVPARFSGSLLGGKAALTLSGAQPEDEAEYYCALWYSNHWVFGGGTKLTVLG

SEQ ID NO: 28 - protein, 115 aa, Artificial Sequence
  52M51-L3 Light chain variable region amino acid
SGVDSQAVVTQEPSLTVSPGGTVTLTCRSSTGAVTTSNYANWFQQKPGQAPRTLIGGTNNRAPGVPARFSGSLLGGKAALTLSGAQPEDEAEYYCALWYSNHWVFGGGTKLTVLG

SEQ ID NO: 29 - DNA, 720 bp, Artificial Sequence
  52M51-L4 Light chain polynucleotide sequence
atgagcgtccctacaatggcttggatgatgctcctgctgggactcctggcttatggaagcggagtggatagccagaccgtcgtcacacaggaacctagcttttccgttagccctggaggaacagtcacactgacctgtaggagctccacaggagctgtgacaacaagcaattacgctaactggtatcagcagactcccggtcaagcccctagaaccctcatcggcggcaccaataacagagctcccggagtccccgacaggttctccggctccatcctgggaaataaagctgctctgacaatcacaggtgcccaggctgacgatgaaagcgactactactgtgcactgtggtacagcaaccattgggttttcggaggcggaacaaagttaaccgtcctcgggcagcctaaggctgctcctagcgtcacactgttccccccatctagcgaggagctgcaggctaacaaggcaaccctcgtctgcctggttagcgacttctaccctggcgctgtcacagtggcctggaaagctgacggctcccctgtgaaagttggcgtcgaaaccacaaagccttctaagcagagcaataataaatatgccgcaagctcctacctctccctgactcctgagcagtggaaaagccataggagctactcctgccgggtcacacacgaaggaagcacagtggaaaagacagtcgcccctgctgagtgtagctga

SEQ ID NO: 30 - protein, 239 aa, Artificial Sequence
  52M51-L4 Light chain amino acid sequence
MSVPTMAWMMLLLGLLAYGSGVDSQTVVTQEPSFSVSPGGTVTLTCRSSTGAVTTSNYANWYQQTPGQAPRTLIGGTNNRAPGVPDRFSGSILGNKAALTITGAQADDESDYYCALWYSNHWVFGGGTKLTVLGQPKAAPSVTLFPPSSEELQANKATLVCLVSDFYPGAVTVAWKADGSPVKVGVETTKPSKQSNNKYAASSYLSLTPEQWKSHRSYSCRVTHEGSTVEKTVAPAECS

SEQ ID NO: 31 - protein, 134 aa, Artificial Sequence
  52M51-L4  Light chain variable region amino acid sequence
MSVPTMAWMMLLLGLLAYGSGVDSQTVVTQEPSFSVSPGGTVTLTCRSSTGAVTTSNYANWYQQTPGQAPRTLIGGTNNRAPGVPDRFSGSILGNKAALTITGAQADDESDYYCALWYSNHWVFGGGTKLTVLG

SEQ ID NO: 32 - protein, 115 aa, Artificial Sequence
  52M51-L4  Light chain variable region amino acid sequence
SGVDSQTVVTQEPSFSVSPGGTVTLTCRSSTGAVTTSNYANWYQQTPGQAPRTLIGGTNNRAPGVPDRFSGSILGNKAALTITGAQADDESDYYCALWYSNHWVFGGGTKLTVLG

SEQ ID NO: 33 - protein, 8 aa, Artificial Sequence
  FLAG-tag
DYKDDDDK

SEQ ID NO: 34 - DNA, 9309 bp, Homo sapiens
atgccgccgctcctggcgcccctgctctgcctggcgctgctgcccgcgctcgccgcacgaggcccgcgatgctcccagcccggtgagacctgcctgaatggcgggaagtgtgaagcggccaatggcacggaggcctgcgtctgtggcggggccttcgtgggcccgcgatgccaggaccccaacccgtgcctcagcaccccctgcaagaacgccgggacatgccacgtggtggaccgcagaggcgtggcagactatgcctgcagctgtgccctgggcttctctgggcccctctgcctgacacccctggacaatgcctgcctcaccaacccctgccgcaacgggggcacctgcgacctgctcacgctgacggagtacaagtgccgctgcccgcccggctggtcagggaaatcgtgccagcaggctgacccgtgcgcctccaacccctgcgccaacggtggccagtgcctgcccttcgaggcctcctacatctgccactgcccacccagcttccatggccccacctgccggcaggatgtcaacgagtgtggccagaagcccgggctttgccgccacggaggcacctgccacaacgaggtcggctcctaccgctgcgtctgccgcgccacccacactggccccaactgcgagcggccctacgtgccctgcagcccctcgccctgccagaacgggggcacctgccgccccacgggcgacgtcacccacgagtgtgcctgcctgccaggcttcaccggccagaactgtgaggaaaatatcgacgattgtccaggaaacaactgcaagaacgggggtgcctgtgtggacggcgtgaacacctacaactgccgctgcccgccagagtggacaggtcagtactgtaccgaggatgtggacgagtgccagctgatgccaaatgcctgccagaacggcgggacctgccacaacacccacggtggctacaactgcgtgtgtgtcaacggctggactggtgaggactgcagcgagaacattgatgactgtgccagcgccgcctgcttccacggcgccacctgccatgaccgtgtggcctccttctactgcgagtgtccccatggccgcacaggtctgctgtgccacctcaacgacgcatgcatcagcaacccctgtaacgagggctccaactgcgacaccaaccctgtcaatggcaaggccatctgcacctgcccctcggggtacacgggcccggcctgcagccaggacgtggatgagtgctcgctgggtgccaacccctgcgagcatgcgggcaagtgcatcaacacgctgggctccttcgagtgccagtgtctgcagggctacacgggcccccgatgcgagatcgacgtcaacgagtgcgtctcgaacccgtgccagaacgacgccacctgcctggaccagattggggagttccagtgcatctgcatgcccggctacgagggtgtgcactgcgaggtcaacacagacgagtgtgccagcagcccctgcctgcacaatggccgctgcctggacaagatcaatgagttccagtgcgagtgccccacgggcttcactgggcatctgtgccagtacgatgtggacgagtgtgccagcaccccctgcaagaatggtgccaagtgcctggacggacccaacacttacacctgtgtgtgcacggaagggtacacggggacgcactgcgaggtggacatcgatgagtgcgaccccgacccctgccactacggctcctgcaaggacggcgtcgccaccttcacctgcctctgccgcccaggctacacgggccaccactgcgagaccaacatcaacgagtgctccagccagccctgccgccacgggggcacctgccaggaccgcgacaacgcctacctctgcttctgcctgaaggggaccacaggacccaactgcgagatcaacctggatgactgtgccagcagcccctgcgactcgggcacctgtctggacaagatcgatggctacgagtgtgcctgtgagccgggctacacagggagcatgtgtaacatcaacatcgatgagtgtgcgggcaacccctgccacaacgggggcacctgcgaggacggcatcaatggcttcacctgccgctgccccgagggctaccacgaccccacctgcctgtctgaggtcaatgagtgcaacagcaacccctgcgtccacggggcctgccgggacagcctcaacgggtacaagtgcgactgtgaccctgggtggagtgggaccaactgtgacatcaacaacaatgagtgtgaatccaacccttgtgtcaacggcggcacctgcaaagacatgaccagtggctacgtgtgcacctgccgggagggcttcagcggtcccaactgccagaccaacatcaacgagtgtgcgtccaacccatgtctgaaccagggcacgtgtattgacgacgttgccgggtacaagtgcaactgcctgctgccctacacaggtgccacgtgtgaggtggtgctggccccgtgtgcccccagcccctgcagaaacggcggggagtgcaggcaatccgaggactatgagagcttctcctgtgtctgccccacgggctggcaagggcagacctgtgaggtcgacatcaacgagtgcgttctgagcccgtgccggcacggcgcatcctgccagaacacccacggcggctaccgctgccactgccaggccggctacagtgggcgcaactgcgagaccgacatcgacgactgccggcccaacccgtgtcacaacgggggctcctgcacagacggcatcaacacggccttctgcgactgcctgcccggcttccggggcactttctgtgaggaggacatcaacgagtgtgccagtgacccctgccgcaacggggccaactgcacggactgcgtggacagctacacgtgcacctgccccgcaggcttcagcgggatccactgtgagaacaacacgcctgactgcacagagagctcctgcttcaacggtggcacctgcgtggacggcatcaactcgttcacctgcctgtgtccacccggcttcacgggcagctactgccagcacgatgtcaatgagtgcgactcacagccctgcctgcatggcggcacctgtcaggacggctgcggctcctacaggtgcacctgcccccagggctacactggccccaactgccagaaccttgtgcactggtgtgactcctcgccctgcaagaacggcggcaaatgctggcagacccacacccagtaccgctgcgagtgccccagcggctggaccggcctttactgcgacgtgcccagcgtgtcctgtgaggtggctgcgcagcgacaaggtgttgacgttgcccgcctgtgccagcatggagggctctgtgtggacgcgggcaacacgcaccactgccgctgccaggcgggctacacaggcagctactgtgaggacctggtggacgagtgctcacccagcccctgccagaacggggccacctgcacggactacctgggcggctactcctgcaagtgcgtggccggctaccacggggtgaactgctctgaggagatcgacgagtgcctctcccacccctgccagaacgggggcacctgcctcgacctccccaacacctacaagtgctcctgcccacggggcactcagggtgtgcactgtgagatcaacgtggacgactgcaatccccccgttgaccccgtgtcccggagccccaagtgctttaacaacggcacctgcgtggaccaggtgggcggctacagctgcacctgcccgccgggcttcgtgggtgagcgctgtgagggggatgtcaacgagtgcctgtccaatccctgcgacgcccgtggcacccagaactgcgtgcagcgcgtcaatgacttccactgcgagtgccgtgctggtcacaccgggcgccgctgcgagtccgtcatcaatggctgcaaaggcaagccctgcaagaatgggggcacctgcgccgtggcctccaacaccgcccgcgggttcatctgcaagtgccctgcgggcttcgagggcgccacgtgtgagaatgacgctcgtacctgcggcagcctgcgctgcctcaacggcggcacatgcatctccggcccgcgcagccccacctgcctgtgcctgggccccttcacgggccccgaatgccagttcccggccagcagcccctgcctgggcggcaacccctgctacaaccaggggacctgtgagcccacatccgagagccccttctaccgttgcctgtgccccgccaaattcaacgggctcttgtgccacatcctggactacagcttcgggggtggggccgggcgcgacatccccccgccgctgatcgaggaggcgtgcgagctgcccgagtgccaggaggacgcgggcaacaaggtctgcagcctgcagtgcaacaaccacgcgtgcggctgggacggcggtgactgctccctcaacttcaatgacccctggaagaactgcacgcagtctctgcagtgctggaagtacttcagtgacggccactgtgacagccagtgcaactcagccggctgcctcttcgacggctttgactgccagcgtgcggaaggccagtgcaaccccctgtacgaccagtactgcaaggaccacttcagcgacgggcactgcgaccagggctgcaacagcgcggagtgcgagtgggacgggctggactgtgcggagcatgtacccgagaggctggcggccggcacgctggtggtggtggtgctgatgccgccggagcagctgcgcaacagctccttccacttcctgcgggagctcagccgcgtgctgcacaccaacgtggtcttcaagcgtgacgcacacggccagcagatgatcttcccctactacggccgcgaggaggagctgcgcaagcaccccatcaagcgtgccgccgagggctgggccgcacctgacgccctgctgggccaggtgaaggcctcgctgctccctggtggcagcgagggtgggcggcggcggagggagctggaccccatggacgtccgcggctccatcgtctacctggagattgacaaccggcagtgtgtgcaggcctcctcgcagtgcttccagagtgccaccgacgtggccgcattcctgggagcgctcgcctcgctgggcagcctcaacatcccctacaagatcgaggccgtgcagagtgagaccgtggagccgcccccgccggcgcagctgcacttcatgtacgtggcggcggccgcctttgtgcttctgttcttcgtgggctgcggggtgctgctgtcccgcaagcgccggcggcagcatggccagctctggttccctgagggcttcaaagtgtctgaggccagcaagaagaagcggcgggagcccctcggcgaggactccgtgggcctcaagcccctgaagaacgcttcagacggtgccctcatggacgacaaccagaatgagtggggggacgaggacctggagaccaagaagttccggttcgaggagcccgtggttctgcctgacctggacgaccagacagaccaccggcagtggactcagcagcacctggatgccgctgacctgcgcatgtctgccatggcccccacaccgccccagggtgaggttgacgccgactgcatggacgtcaatgtccgcgggcctgatggcttcaccccgctcatgatcgcctcctgcagcgggggcggcctggagacgggcaacagcgaggaagaggaggacgcgccggccgtcatctccgacttcatctaccagggcgccagcctgcacaaccagacagaccgcacgggcgagaccgccttgcacctggccgcccgctactcacgctctgatgccgccaagcgcctgctggaggccagcgcagatgccaacatccaggacaacatgggccgcaccccgctgcatgcggctgtgtctgccgacgcacaaggtgtcttccagatcctgatccggaaccgagccacagacctggatgcccgcatgcatgatggcacgacgccactgatcctggctgcccgcctggccgtggagggcatgctggaggacctcatcaactcacacgccgacgtcaacgccgtagatgacctgggcaagtccgccctgcactgggccgccgccgtgaacaatgtggatgccgcagttgtgctcctgaagaacggggctaacaaagatatgcagaacaacagggaggagacacccctgtttctggccgcccgggagggcagctacgagaccgccaaggtgctgctggaccactttgccaaccgggacatcacggatcatatggaccgcctgccgcgcgacatcgcacaggagcgcatgcatcacgacatcgtgaggctgctggacgagtacaacctggtgcgcagcccgcagctgcacggagccccgctggggggcacgcccaccctgtcgcccccgctctgctcgcccaacggctacctgggcagcctcaagcccggcgtgcagggcaagaaggtccgcaagcccagcagcaaaggcctggcctgtggaagcaaggaggccaaggacctcaaggcacggaggaagaagtcccaggacggcaagggctgcctgctggacagctccggcatgctctcgcccgtggactccctggagtcaccccatggctacctgtcagacgtggcctcgccgccactgctgccctccccgttccagcagtctccgtccgtgcccctcaaccacctgcctgggatgcccgacacccacctgggcatcgggcacctgaacgtggcggccaagcccgagatggcggcgctgggtgggggcggccggctggcctttgagactggcccacctcgtctctcccacctgcctgtggcctctggcaccagcaccgtcctgggctccagcagcggaggggccctgaatttcactgtgggcgggtccaccagtttgaatggtcaatgcgagtggctgtcccggctgcagagcggcatggtgccgaaccaatacaaccctctgcgggggagtgtggcaccaggccccctgagcacacaggccccctccctgcagcatggcatggtaggcccgctgcacagtagccttgctgccagcgccctgtcccagatgatgagctaccagggcctgcccagcacccggctggccacccagcctcacctggtgcagacccagcaggtgcagccacaaaacttacagatgcagcagcagaacctgcagccagcaaacatccagcagcagcaaagcctgcagccgccaccaccaccaccacagccgcaccttggcgtgagctcagcagccagcggccacctgggccggagcttcctgagtggagagccgagccaggcagacgtgcagccactgggccccagcagcctggcggtgcacactattctgccccaggagagccccgccctgcccacgtcgctgccatcctcgctggtcccacccgtgaccgcagcccagttcctgacgcccccctcgcagcacagctactcctcgcctgtggacaacacccccagccaccagctacaggtgcctgagcaccccttcctcaccccgtcccctgagtcccctgaccagtggtccagctcgtccccgcattccaacgtctccgactggtccgagggcgtctccagccctcccaccagcatgcagtcccagatcgcccgcattccggaggccttcaagtaaacggcgcgccccacgagaccccggcttcctttcccaagccttcgggcgtctgtgtgcgctctgtggatgccagggccgaccagaggagcctttttaaaacacatgtttttatacaaaataagaacgaggattttaattttttttagtatttatttatgtacttttattttacacagaaacactgcctttttatttatatgtactgttttatctggccccaggtagaaacttttatctattctgagaaaacaagcaagttctgagagccagggttttcctacgtaggatgaaaagattcttctgtgtttataaaatataaacaaagattcatgatttataaatgccatttatttattgattccttttttcaaaatccaaaaagaaatgatgttggagaagggaagttgaacgagcatagtccaaaaagctcctggggcgtccaggccgcgccctttccccgacgcccacccaaccccaagccagcccggccgctccaccagcatcacctgcctgttaggagaagctgcatccagaggcaaacggaggcaaagctggctcaccttccgcacgcggattaatttgcatctgaaataggaaacaagtgaaagcatatgggttagatgttgccatgtgttttagatggtttcttgcaagcatgcttgtgaaaatgtgttctcggagtgtgtatgccaagagtgcacccatggtaccaatcatgaatctttgtttcaggttcagtattatgtagttgttcgttggttatacaagttcttggtccctccagaaccaccccggccccctgcccgttcttgaaatgtaggcatcatgcatgtcaaacatgagatgtgtggactgtggcacttgcctgggtcacacacggaggcatcctacccttttctggggaaagacactgcctgggctgaccccggtggcggccccagcacctcagcctgcacagtgtcccccaggttccgaagaagatgctccagcaacacagcctgggccccagctcgcgggacccgaccccccgtgggctcccgtgttttgtaggagacttgccagagccgggcacattgagctgtgcaacgccgtgggctgcgtcctttggtcctgtccccgcagccctggcagggggcatgcggtcgggcaggggctggagggaggcgggggctgcccttgggccacccctcctagtttgggaggagcagatttttgcaataccaagtatagcctatggcagaaaaaatgtctgtaaatatgtttttaaaggtggattttgtttaaaaaatcttaatgaatgagtctgttgtgtgtcatgccagtgagggacgtcagacttggctcagctcggggagccttagccgcccatgcactggggacgctccgctgccgtgccgcctgcactcctcagggcagcctcccccggctctacgggggccgcgtggtgccatccccagggggcatgaccagatgcgtcccaagatgttgatttttactgtgttttataaaatagagtgtagtttacagaaaaagactttaaaagtgatctacatgaggaactgtagatgatgtatttttttcatcttttttgttaactgatttgcaataaaaatgatactgatggtgaaaaaaaaaaaaaaa

(19) United States
(12) Patent Application Publication
(10) Pub. No.: US 2015/0232570 A1
(43) Pub. Date: Aug. 20, 2015

(54) Methods of Treating Hematological Malignancies with Notch1 Antibodies

(71) Applicant: Oncomed Pharmaceuticals, Inc., Redwood City, CA (US)

(72) Inventors: Timothy C. Hoey, Hillsborough, CA (US); Ann M. Kapoun, Mountain View, CA (US); Min Wang, Burlingame, CA (US); Tzu-Ling Tang Lin, Redwood City, CA (US); Jennifer Anne Cain, Redwood City, CA (US); Jakob Dupont, Hillsborough, CA (US)

(73) Assignee: ONCOMED PHARMACEUTICALS, INC., Redwood City, CA (US)

(21) Appl. No.: 14/429,497

(22) PCT Filed: Sep. 20, 2013
(86) PCT No.: PCT/US2013/060878

§ 371 Date: Mar. 19, 2015

Related U.S. Application Data
(60) Provisional application No. 61/782,411, filed on Mar. 14, 2013.
(60) Provisional application No. 61/726,182, filed on Nov. 14, 2012.
(60) Provisional application No. 61/704,016, filed on Sep. 21, 2012.

(51) Int. Cl. C07K 16/30; A61K 45/06; C12Q 1/68; A61K 39/395

(52) U.S. Cl. CPC C07K 16/3061; A61K 39/39558; A61K 45/06; C12Q 1/6886; C07K 2317/565; C07K 2317/24; C07K 2317/73; C07K 2317/76; A61K 2039/505

(57) ABSTRACT

NOTCH1-binding antibodies and methods of using the antibodies for treating hematologic cancers are disclosed. Methods of using antibodies that bind to a non-ligand binding membrane proximal region of the extracellular domain of human NOTCH1 and methods of treating chronic lymphocytic leukemia (CLL), hairy cell leukemia, chronic myelogenous leukemia (CML), nonHodgkin lymphoma, diffuse large B-cell lymphoma (DLBCL), mantle cell lymphoma (MCL), or cutaneous T-cell lymphoma (CTCL) in a subject, comprising administering a therapeutically effective amount of a NOTCH1-binding antibody to the subject are also disclosed. Methods of identifying subjects suitable for such treatment methods are further disclosed.

### CROSS REFERENCE TO RELATED APPLICATIONS

This application claims the priority benefit of U.S. Provisional Application No. 61/704,016, filed Sep. 21, 2012, U.S. Provisional Application No. 61/726,182, filed Nov. 14, 2012, and U.S. Provisional Application No. 61/782,411, filed Mar. 14, 2013, each of which is hereby incorporated by reference herein in its entirety.

### FIELD OF THE INVENTION

The field of this invention generally relates to methods of using antibodies that bind human NOTCH1 for the treatment of hematologic diseases, as well as methods of selecting patients for such treatment.

### BACKGROUND OF THE INVENTION

The NOTCH signaling pathway is a universally conserved signal transduction system. It is involved in cell fate determination during development including embryonic pattern formation and post-embryonic tissue maintenance. In addition, NOTCH signaling has been identified as a critical factor in the maintenance of hematopoietic stem cells.

The mammalian NOTCH receptor family includes four members, NOTCH1, NOTCH2, NOTCH3 and NOTCH4. NOTCH receptors are large single-pass type I transmembrane proteins with several conserved structural motifs. The extracellular domain contains a variable number of epidermal growth factor (EGF)-like repeats involved in ligand binding and three cysteine-rich LIN-12/NOTCH repeats (LNRs) involved in NOTCH heterodimerization. The intracellular domain contains a RAM23 motif involved in binding NOTCH downstream signaling proteins, 7 cdc10/ankyrin repeats also involved in mediating downstream signaling and a PEST domain involved in NOTCH protein degradation.

Mammalian NOTCH ligands include Delta-like 1 (DLL1), Delta-like 3 (DLL3), Delta-like 4 (DLL4), Jagged1 and Jagged2. Similar to NOTCH receptors, NOTCH ligands are type I transmembrane proteins with several conserved structural motifs. Extracellular motifs common to all NOTCH ligands include a single Delta/Serrate/Lag-2 (DSL) domain involved in receptor binding, as well as a variable number of EGF-like repeats that may be involved in stabilizing receptor binding. The extracellular domain of Jagged proteins contains a cysteine-rich region which has partial homology to the von Willebrand factor type C domain and is likely involved in ligand dimerization. This motif is not present in DLL family members. (Leong et al., 2006, Blood, 107:2223-2233).

The extracellular domain of a NOTCH receptor interacts with the extracellular domain of a NOTCH ligand, typically on adjacent cells, resulting in two proteolytic cleavages of the NOTCH receptor. One extracellular cleavage is mediated by an ADAM (A Disintegrin And Metallopeptidase) protease and a second cleavage within the transmembrane domain is mediated by the gamma secretase complex. This latter cleavage generates the NOTCH intracellular domain (ICD), which translocates to the nucleus where it activates the CBF1, Suppressor of Hairless, Lag-2 (CSL) family of transcription factors as the major downstream effectors to increase transcription of nuclear basic helix-loop-helix transcription factors of the Hairy/Enhancer of Split (HES) family. (Artavanis et al., 1999, Science, 284:770; Brennan and Brown, 2003, Breast Cancer Res., 5:69; Iso et al., 2003, Arterioscler. Thromb. Vasc. Biol., 23:543).

The NOTCH pathway has been linked to the pathogenesis of both hematologic and solid tumors and cancers. Numerous cellular functions and microenvironmental cues associated with tumorigenesis have been shown to be modulated by NOTCH pathway signaling, including cell proliferation, apoptosis, adhesion, and angiogenesis. (Leong et al., 2006, Blood, 107:2223-2233). In addition, NOTCH receptors and/or NOTCH ligands have been shown to play potential oncogenic roles in a number of human cancers, including acute myelogenous leukemia, B cell chronic lymphocytic leukemia, Hodgkin lymphoma, multiple myeloma, T-cell acute lymphoblastic leukemia, brain cancer, breast cancer, cervical cancer, colon cancer, lung cancer, pancreatic cancer, prostate cancer and skin cancer. (Leong et al., 2006, Blood, 107:2223-2233).

The NOTCH1 gene in humans was first identified in a subset of T-cell acute lymphoblastic leukemias as a translocated locus resulting in activation of the NOTCH pathway (Ellisen et al., 1991, Cell, 66:649-61). It has been shown that more than 50% of human T-cell acute lymphoblastic leukemias have activating mutations that involve the extracellular heterodimerization domain and/or the C-terminal PEST domain of NOTCH1 (Weng et al., 2004, Science, 306:269-271; Pear & Aster, 2004, Curr. Opin. Hematol., 11:416-33). Constitutive activation of NOTCH1 signaling in T-cells in mouse models similarly generates T-cell lymphomas suggesting a causative role (Robey et al., 1996, Cell, 87:483-92; Pear et al., 1996, J. Exp. Med., 183:2283-91; Yan et al., 2001, Blood, 98:3793-9; Bellavia et al., 2000, EMBO J., 19:3337-48). Retrovirally-activated NOTCH2 has been implicated in thymic lymphoma induced by feline leukemia virus (Rohn et al., 1996, J. Virology, 70:8071-8080). Human T-cell acute lymphoblastic leukemia samples have been shown to express NOTCH3 and its target gene HES-1, which were not expressed in normal peripheral T-cells nor in non-T-cell leukemias (Bellavia et al., 2002, PNAS, 99:3788-3793). More recently, constitutive activation of NOTCH1 signaling has been reported in chronic lymphocytic leukemia and sequencing technologies have shown the some CLL patients harbor NOTCH1 mutations (Rosati et al, 2009, Blood, 113:856-865; Di lanni et al., 2009, Brit. J. Haem., 146:689-691; Fabbri et al., 2011, JEM, 208:1389-1401; Gianfelici, 2012, Haematologica, 97:328-330). Thus, the NOTCH pathway has been identified as a potential target for therapeutic intervention in several hematologic cancers.

Anti-NOTCH antibodies and their possible use as anti-cancer therapeutics have been reported. See, e.g., U.S. Patent Application Publication Nos. 2008/0131434 and 2009/0081238. See also International Publication Nos. WO 2008/057144, WO 2008/076960, WO 2008/150525, WO 2010/005566 and WO 2010/005567.

### SUMMARY OF THE INVENTION

The present invention provides methods of using antibodies that bind human NOTCH1 for the treatment of hematologic cancers. In some embodiments, the antibodies bind the non-ligand binding membrane proximal region of the extracellular domain of the human NOTCH1 receptor. The invention further provides methods of selecting subjects having hematologic cancers for treatment with the NOTCH1-binding antibodies. In some embodiments, the hematologic cancer is a leukemia or lymphoma. In some embodiments the hematologic cancer is chronic lymphocytic leukemia (CLL), hairy cell leukemia, chronic myelogenous leukemia (CML), non-Hodgkin lymphoma, diffuse large B-cell lymphoma (DLBCL), mantle cell lymphoma (MCL), or cutaneous T-cell lymphoma (CTCL). In some embodiments, NOTCH1 is activated in the hematologic cancer.

In one aspect, the invention provides methods of treating a hematologic cancer in a human subject comprising administering to the subject a therapeutically effective amount of an antibody that binds human NOTCH1.

In another aspect, the invention provides methods of inhibiting the growth of hematologic cancer cells comprising contacting the cancer cells with an effective amount of an antibody that binds human NOTCH1.

In certain embodiments of each of the aforementioned aspects or embodiments, as well as other aspects and/or embodiments described elsewhere herein, the hematologic cancer comprises cancer cells in which NOTCH1 is activated. In certain embodiments, the hematologic cancer comprises a NOTCH1 mutation, which may be an activating NOTCH1 mutation. By way of non-limiting example, the mutation may be in the sequence of the NOTCH1 gene that encodes the PEST domain and/or the HD domain of NOTCH1. In certain embodiments, the mutation is a truncation mutation in the PEST domain. In additional embodiments, the mutation may be in the sequence of the NOTCH1 gene that encodes an EGF repeat of the NOTCH extracellular domain. In additional embodiments, the mutation is in EGF repeat 36 of the NOTCH1 extracellular domain.

In a further aspect, the invention provides methods of treating a hematologic cancer in a human subject, comprising (a) determining that the subject's hematologic cancer comprises a NOTCH1 mutation, and (b) administering to the subject a therapeutically effective amount of an antibody that binds human NOTCH1.

In still another aspect, the invention provides methods of treating a hematologic cancer in a human subject, comprising (a) selecting a subject for treatment with an antibody that binds human NOTCH1 based, at least in part, on the subject having a hematologic cancer that comprises a NOTCH1 mutation, and (b) administering to the subject a therapeutically effective amount of the antibody.

In a farther aspect, the invention provides methods of treating a hematologic cancer in a human subject, comprising (a) identifying a subject that has a hematologic cancer comprising a NOTCH1 mutation, and (b) administering to the subject a therapeutically effective amount of an antibody that binds human NOTCH1.

In a further aspect, the invention provides methods of treating a hematologic cancer in a human subject, comprising (a) determining that NOTCH1 is activated in the subject's hematologic cancer, and (b) administering to the subject a therapeutically effective amount of an antibody that binds human NOTCH1.

In still another aspect, the invention provides methods of treating a hematologic cancer in a human subject, comprising (a) selecting a subject for treatment with an antibody that binds human NOTCH1 based, at least in part, on the subject having a hematologic cancer in which NOTCH1 is activated, and (b) administering to the subject a therapeutically effective amount of the antibody.

In a further aspect, the invention provides methods of treating a hematologic cancer in a human subject, comprising (a) identifying a subject that has a hematologic cancer in which NOTCH1 is activated, and (b) administering to the subject a therapeutically effective amount of an antibody that binds human NOTCH1.

In an additional aspect, the invention provides methods of selecting a human subject having a hematologic cancer for treatment with an antibody that binds to human NOTCH1, comprising determining whether the subject has a hematologic cancer that has a NOTCH1 mutation (or has cancer cells that have a NOTCH1 mutation), wherein if the cancer (or cells) have a NOTCH1 mutation, the subject is selected for treatment with the antibody.

In certain embodiments of each of the aforementioned aspects or embodiments, as well as other aspects and/or embodiments described elsewhere herein, the activation of NOTCH1 is determined or identified by detecting the presence of a NOTCH1 mutation in the subject's cancer cells (e.g., in a sample obtained from the subject). In certain embodiments, the presence of a NOTCH1 mutation is determined by sequencing (e.g., DNA amplification followed by sequencing or direct sequencing). In some alternative embodiments, the activation of NOTCH1 is determined by detecting elevated levels of the NOTCH1 intracellular domain (ICD) in the nucleus of the cancer cells (e.g., by NOTCH ICD IHC assay).

In an additional aspect, the invention provides methods of selecting a human subject having a hematologic cancer for treatment with an antibody that binds to human NOTCH1, comprising determining whether the subject has a hematologic cancer in which NOTCH1 is activated, wherein if NOTCH1 is activated in the hematologic cancer, the subject is selected for treatment with the antibody.

In certain embodiments of each of the aforementioned aspects or embodiments, as well as other aspects and/or embodiments described elsewhere herein, the NOTCH1 mutation is an activating NOTCH1 mutation. By way of non-limiting example, the mutation may be in the PEST domain and/or the HD domain of the NOTCH1 gene. In certain embodiments, the mutation is a truncation mutation in the PEST domain. In additional embodiments, the mutation is in an EGF repeat of the NOTCH extracellular domain. In additional embodiments, the mutation is in EGF repeat 36 of the NOTCH1 extracellular domain.

In certain embodiments of each of the aforementioned aspects or embodiments, as well as other aspects and/or embodiments described elsewhere herein, the methods further comprise a step of obtaining a body sample from the subject which is used to determine or identify the patient as having a hematologic cancer in which NOTCH1 is activated and/or the NOTCH1 gene is mutated (e.g., has an activating NOTCH1 mutation). In certain embodiments, the sample is whole blood, serum, plasma, or tissue.

In certain embodiments of each of the aforementioned aspects or embodiments, as well as other aspects and/or embodiments described elsewhere herein, the hematologic cancer is chronic lymphocytic leukemia (CLL), hairy cell leukemia, chronic myelogenous leukemia (CML), non-Hodgkin lymphoma, diffuse large B-cell lymphoma (DLBCL), mantle cell lymphoma (MCL), or cutaneous T-cell lymphoma (CTCL). In some embodiments, the hematologic cancer is chronic lymphocytic leukemia. In some embodiments, the hematologic cancer is mantle cell lymphoma. In some embodiments, the hematologic cancer is non-Hodgkin lymphoma. In some embodiments, the hematologic cancer is a cutaneous T-cell lymphoma. In some embodiments, the hematologic cancer is mycosis fungoides (a form of cutaneous T-cell lymphoma). In some embodiments, the hematologic cancer is transformed mycosis fungoides. In some embodiments, the hematologic cancer is Sézary Syndrome (a form of cutaneous T-cell lymphoma). In some embodiments, the subject to be treated has developed or is developing Richter's transformation (also referred to herein as Richter's syndrome). Alternatively, the subject to be treated has CLL and is at risk of developing Richter's transformation. Other non-limiting examples of hematologic cancers which may be treated using the NOTCH1-binding antibodies and methods provided herein include NK-cell leukemia, splenic marginal zone lymphoma, and follicular lymphoma. In some embodiments, the hematologic cancer may be refractory.

In certain embodiments of each of the aforementioned aspects or embodiments, as well as other aspects and/or embodiments described elsewhere herein, the NOTCH1-binding antibody binds the extracellular domain of human NOTCH1. In some embodiments, the antibody binds a non-ligand binding membrane proximal region of the extracellular domain of a human NOTCH1 receptor. In certain embodiments, the non-ligand binding membrane proximal region of a NOTCH1 receptor comprises about amino acid 1427 to about amino acid 1732 of a human NOTCH1 receptor. In some embodiments, the membrane proximal region of a NOTCH1 receptor bound by the antibody comprises at least a portion of SEQ ID NO:2. In some embodiments, the membrane proximal region of a NOTCH1 receptor bound by the antibody comprises SEQ ID NO:2.

In certain embodiments of each of the aforementioned aspects or embodiments, as well as other aspects and/or embodiments described elsewhere herein, the NOTCH1-binding antibody comprises: (a) a heavy chain CDR1 comprising RGYWIE (SEQ ID NO:15), or a variant thereof comprising 1, 2, 3, or 4 amino acid substitutions; (b) a heavy chain CDR2 comprising QILPGTGRTNYNEKFKG (SEQ ID NO:16), or a variant thereof comprising 1, 2, 3, or 4 amino acid substitutions; and (c) a heavy chain CDR3 comprising FDGNYGYYAMDY (SEQ ID NO:17), or a variant thereof comprising 1, 2, 3, or 4 amino acid substitutions. In certain embodiments, the antibody comprises (or further comprises) (a) a light chain CDR1 comprising RSSTGAVTTSNYAN (SEQ ID NO:18), or a variant thereof comprising 1, 2, 3, or 4 amino acid substitutions; (b) a light chain CDR2 comprising GTNNRAP (SEQ ID NO:19), or a variant thereof comprising 1, 2, 3, or 4 amino acid substitutions; and (c) a light chain CDR3 comprising ALWYSNHWVFGGGTKL (SEQ ID NO:20), or a variant thereof comprising 1, 2, 3, or 4 amino acid substitutions. In some embodiments, the antibody comprises (a) a heavy chain CDR1 comprising RGYWIE (SEQ ID NO:15), a heavy chain CDR2 comprising QILPGTGRTNYNEKFKG (SEQ ID NO:16), and a heavy chain CDR3 comprising FDGNYGYYAMDY (SEQ ID NO:17); and/or (b) a light chain CDR1 comprising RSSTGAVTTSNYAN (SEQ ID NO: 18), a light chain CDR2 comprising GTNNRAP (SEQ ID NO:19), and a light chain CDR3 comprising ALWYSNHWVFGGGTKL (SEQ ID NO:20).

In certain embodiments of each of the aforementioned aspects or embodiments, as well as other aspects and/or embodiments described elsewhere herein, the NOTCH1-binding antibody comprises: (a) a heavy chain variable region having at least about 90%, at least about 95%, at least about 98%, or 100% sequence identity to SEQ ID NO:14 or SEQ ID NO:24; and (b) a light chain variable region having at least about 90%, at least about 95%, at least about 98%, or 100% sequence identity to SEQ ID NO:8, SEQ ID NO:28, or SEQ ID NO:32. In some embodiments, the antibody comprises a heavy chain variable region comprising SEQ ID NO: 14 and a light chain variable region comprising SEQ ID NO:8. In some embodiments, the antibody comprises a heavy chain variable region comprising SEQ ID NO:24 and a light chain variable region comprising SEQ ID NO:28. In some embodiments, the antibody comprises a heavy chain variable region comprising SEQ ID NO:24 and a light chain variable region comprising SEQ ID NO:32. In some embodiments, the NOTCH1-binding antibody is a humanized form of 52M51, the antibody produced by the hybridoma deposited with the American Type Culture Collection (ATCC), 10801 University Boulevard, Manassas, Va., USA, under the conditions of the Budapest Treaty on Aug. 7, 2008, and assigned designation number PTA-9405. In some embodiments, the antibody is a humanized version of antibody 52M51, 52M51-H4L3, as encoded by the polynucleotide deposited with the ATCC, under the conditions of the Budapest Treaty on Oct. 15, 2008, and assigned designation number PTA-9549. In certain embodiments, the NOTCH1-binding antibody comprises the heavy chains and light chains of the 52M51 antibody or 52M51-H4L3 antibody (with or without the signal/leader sequence).

In certain embodiments of each of the aforementioned aspects or embodiments, as well as other aspects and/or embodiments described elsewhere herein, the NOTCH1-binding antibody used in the methods binds the same epitope on human NOTCH1 as 52M51 (or another NOTCH1-binding antibody described herein) binds, or an epitope on human NOTCH1 that overlaps with the epitope on human NOTCH1 that 52M51 (or another NOTCH1-binding antibody described herein) binds.

In certain embodiments of each of the aforementioned aspects or embodiments, as well as other aspects and/or embodiments described elsewhere herein, the antibody competes with antibody 52M51 (or another NOTCH1-binding antibody provided herein) for binding to human NOTCH1 (e.g., to the membrane proximal region of NOTCH1).

In certain embodiments of each of the aforementioned aspects or embodiments, as well as other aspects and/or embodiments described elsewhere herein, the antibody is a recombinant antibody. In some embodiments, the antibody is a monoclonal antibody. In some embodiments, the antibody is a chimeric antibody. In some embodiments, the antibody is a humanized antibody. In some embodiments, the antibody is a human antibody. In certain embodiments, the antibody is an antibody fragment. In certain embodiments, the antibody or antibody fragment is monovalent, monospecific, bivalent, bispecific, or multispecific. In certain embodiments, the antibody is conjugated to a cytotoxic moiety. In certain embodiments, the antibody is isolated. In still further embodiments, the antibody is substantially pure.

In certain embodiments of each of the aforementioned aspects or embodiments, as well as other aspects and/or embodiments described elsewhere herein, the NOTCH1-binding antibody is an antagonist of NOTCH1. In some embodiments, the antibody inhibits NOTCH1 signaling. In some embodiments, the antibody inhibits NOTCH1 activation. In some embodiments, the antibody inhibits activity of a constitutively activated NOTCH1. In some embodiments, the antibody inhibits cleavage within the membrane proximal region. In certain embodiments, the antibody inhibits cleavage of NOTCH1 (e.g., cleavage at the S2 site by a metalloprotease) and/or inhibits activation of NOTCH1 by ligand binding. In some embodiments, the antibody inhibits release or formation of the intracellular domain (ICD) of NOTCH1. In certain embodiments, the antibody inhibits growth of hematologic cancer cells. In some embodiments, the antibody inhibits growth of chronic lymphocytic leukemia cells.

In some embodiments, the methods comprise targeting cancer stem cells with the antibodies described herein. In certain embodiments, the methods comprise reducing the frequency of cancer stem cells in a hematologic cancer, reducing the number of cancer stem cells in a hematologic cancer, reducing the tumorigenicity of a hematologic cancer, and/or reducing the tumorigenicity of a hematologic cancer by reducing the number or frequency of cancer stem cells in the hematologic cancer.

In certain embodiments of each of the aforementioned aspects and/or embodiments, as well as other aspects or embodiments described herein, the methods further comprise administering to the subject at least one additional therapeutic agent or therapy. In certain embodiments of each of the aforementioned aspects or embodiments, as well as other aspects and/or embodiments described elsewhere herein, the antibody is administered to a subject in combination with at least one additional treatment for a hematologic cancer. In certain embodiments, the additional treatment for a hematologic cancer comprises radiation therapy, chemotherapy, immunotherapy, targeted therapy, surgery, stem cell transplant, photodynamic therapy, ultraviolet B radiation therapy, donor lymphocyte infusion (DLI) and/or an additional antibody therapeutic. Thus, the invention further provides a method of treating a hematologic cancer in a human comprising administering to the human therapeutically effective amounts of (a) an antibody that binds NOTCH1; and (b) at least one additional therapeutic agent or therapy.

In certain embodiments of each of the aforementioned aspects and/or embodiments, as well as other aspects or embodiments described herein, the human subject to which the antibody is administered has previously failed a cancer therapy.

The invention further provides methods of selecting a human subject having diffuse large B-cell lymphoma (DLBCL) for treatment with an antibody that binds to human NOTCH1 and inhibits NOTCH1 activation or signaling, comprising determining whether cancer cells from the subject contain a deletion at position 7444 of the human NOTCH1 gene or a substitution at position 4168 of the human NOTCH1 gene (e.g., C4168A, C4168G, and C4168T substitution), and selecting the subject whose cancer cells or cancer comprises the mutation for treatment with the antibody.

Methods of treating a human subject having DLBCL comprising an activating NOTCH1 mutation are further provided. In some embodiments, such methods comprise administering to the subject a therapeutically effective amount of an antibody that binds to human NOTCH1 and inhibits activation or signaling of NOTCH1. In some embodiments, the DLBCL comprises a deletion at position 7444 of the human NOTCH1 gene. In some embodiments, the DLBCL comprises a substitution at position 4168 of the human NOTCH1 gene (e.g., C4168A, C4168G, and C4168T substitution).

The invention also provides methods of treating a human subject having DLBCL, comprising determining that cancer cells from the subject comprise and activating NOTCH1 mutation, and administering to the subject a therapeutically effective amount of an antibody that binds to human NOTCH1 and inhibits activation or signaling of NOTCH1. In certain embodiments, the activating mutation comprises a deletion at position 7444 of NOTCH1. In certain embodiments, the activating mutation comprises a substitution at position 4168 of the human NOTCH1 gene (e.g., C4168A, C4168G, and C4168T substitution). In certain embodiments, the methods further comprise a step of obtaining a body sample from the subject which is used to determine whether DLBCL cells in the subject contain the mutation. In certain embodiments, the sample is whole blood, serum, plasma, or tissue. In certain embodiments, determining the presence of the mutation comprises sequencing.

In another aspect, the invention provides isolated polynucleotides comprising a sequence encoding a mutant human NOTCH1 receptor. In certain embodiments, the polynucleotides comprise a deletion at position 7444 of the human NOTCH1 gene. In certain embodiments, the activating mutation comprises a substitution at position 4168 of the human NOTCH1 gene (e.g., C4168A, C4168G, and C4168T substitution). Isolated polypeptides encoded by the polynucleotides are provided. Vectors comprising the polynucleotides (e.g., operably linked to a promoter sequence) and cells comprising the vectors are also provided.

Where aspects or embodiments of the invention are described in terms of a Markush group or other grouping of alternatives, including, but not limited to, groups of alternatives separated by “and/or” or “or,” the present invention encompasses not only the entire group listed as a whole, but each member of the group individually and all possible subgroups of the main group, but also the main group absent one or more of the group members. The present invention also envisages the explicit exclusion of one or more of any of the group members in the claimed invention. For example, language such as “X and/or Y” encompasses “X” individually, “Y” individually, as well as “X” and “Y” together.

### BRIEF DESCRIPTION OF THE DRAWINGS

FIG. 1: Identification of Antibodies Targeting the Membrane Proximal Region of NOTCH1 that Inhibit NOTCH Signaling. (A) Schematic of the NOTCH receptor and 52M antigen region. The 52M antigen includes the area of the NOTCH1 receptor subject to cleavage by furin during maturation of the receptor and cleavage by ADAM (A Disintegrin and Metalloprotease) proteases following ligand binding. Subsequent processing by gamma-secretase causes the release of the intracellular domain (ICD) of NOTCH1 that activates gene transcription in the nucleus. (B) Luciferase levels (y-axis) derived from NOTCH1-HeLa cells cultured in the presence of a soluble NOTCH ligand (hDLL4-Fc) and NOTCH1 antibodies. Results from non-transfected (NT) cells with and without hDLL4-Fc are shown on the far left of the x-axis. 52M NOTCH1 antibodies are shown along the x-axis and compared to DBZ, a NOTCH gamma-secretase inhibitor, and 21M18, an anti-DLL4 antibody. (C) Luciferase levels (y-axis) derived from NOTCH1-HeLa cells cultured in the presence of a soluble NOTCH ligand (hDLL4-Fc) and NOTCH1 receptor antibodies. Results from non-transfected (NT) cells with and without hDLL4-Fe are shown on the far left of the x-axis, 52M5 I murine hybridoma-derived antibody and humanized variant 52M51-H4L3 are shown along the x-axis in various concentrations as indicated. (D) Western blot analysis of ICD formation after ligand-mediated stimulation of NOTCH1-expressing HeLa cells.

FIG. 2: Validation of NOTCH1 L2482X mutation in DLBCL sample DLBCL-N020029 by Sanger sequencing.

FIG. 3: In vitro activity of L2482X NOTCH1 mutant in luciferase reporter assay.

FIG. 4: 52M51 anti-NOTCH1 antibody inhibits the activity of L2482X and P2514fs NOTCH1 mutant polypeptide as measured in a luciferase reporter assay. FIGS. 4A and B show the Firefly luciferase to Renilla luciferase activity ratio observed in L2482X NOTCH1_mutant polypeptide expressing PC3 cells after stimulation with DLL4 and JAG1, respectively, in the presence of increasing concentrations of 52M51 antibody. FIGS. 4C and D show the Firefly luciferase to Renilla luciferase activity ratio observed in P2514fs NOTCH1_mutant polypeptide expressing PC3 cells after stimulation with DLL4 and JAG1, respectively, in the presence of increasing concentrations of 52M5 antibody. All of FIGS. 4A-D also includes data obtained with control PC3 cells that did not express a recombinant NOTCH1 polypeptide.

FIG. 5: Validation of NOTCH1 P1390T mutation in DLBCL sample DLBCL_{—}10000487 by Sanger sequencing.

FIG. 6: Anti-NOTCH1 antibody (52M51) treatment reduces viability of REC1 mantle cell lymphoma cells.

FIG. 7: Viability of NOTCH1ΔPEST mutant (A-D) and NOTCH1 wild type (E-F) primary CLL cells following in vitro treatment with 52M51 anti-NOTCH1 antibody in the presence of recombinant human DLL4 protein.

### DETAILED DESCRIPTION OF THE INVENTION

The present invention provides novel methods of treatment for hematologic cancers and novel methods of inhibiting the growth of hematologic cancers with antibodies that bind human NOTCH1. The NOTCH1-binding antibodies include antagonists of human NOTCH1. Methods of selecting patients for treatment with the antibodies are also provided and, in some embodiments, comprise determining whether NOTCH1 is activated in the hematologic cancers which afflict the patients and/or whether the hematologic cancer cells from the patient comprise a NOTCH1 mutation.

In some embodiments, the antibodies bind to a non-ligand binding membrane proximal region of the extracellular domain of human NOTCH1 and inhibit tumor growth in vivo. The ligand binding region of NOTCH, which is necessary and sufficient for ligand binding, has been identified as EGF repeats 11 and 12, suggesting this region of the NOTCH receptor is important in NOTCH signaling and tumorigenesis (Rebay et al., 1991, Cell, 67:687; Lei et al., 2003, Dev., 130:6411; Hambleton et al., 2004, Structure, 12:2173). Unexpectedly, antibodies that bind outside the ligand binding domain of the extracellular domain of human NOTCH receptors have been found to inhibit tumor cell growth in vivo (see U.S. Patent Pub. No. 2008/0131434 and International Pub. Nos. WO 2010/005567 and WO 2011/088215). Thus, antibodies that bind outside the ligand binding domain of the extracellular domain of one or more of the human NOTCH receptors—NOTCH1, NOTCH2, NOTCH3, and NOTCH4—have value as potential cancer therapeutics.

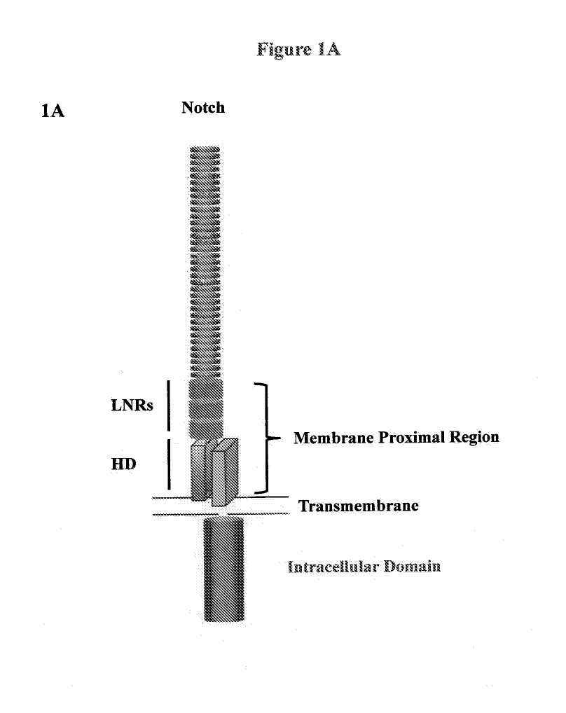

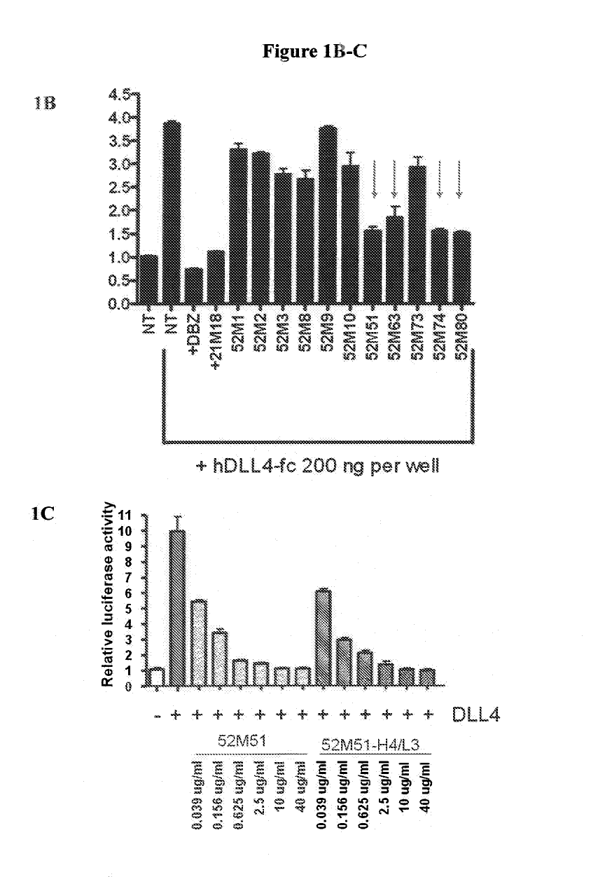

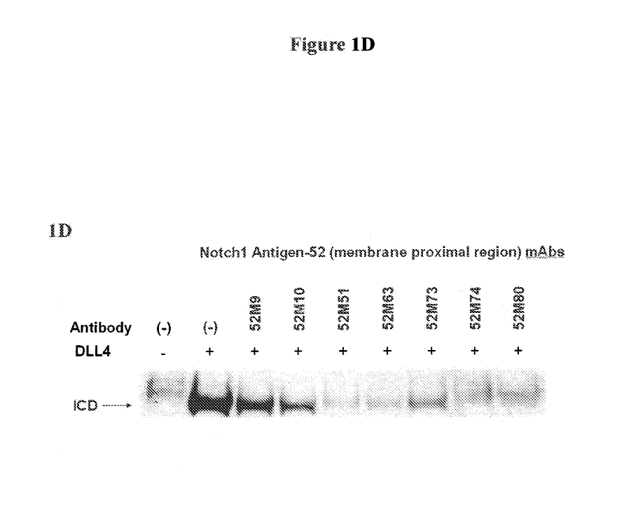

Monoclonal antibodies that specifically bind to the membrane proximal region of the extracellular domain of human NOTCH1, including the monoclonal antibody 52M51, have been identified (Example 1). Humanized 52M51 antibodies have been generated (Example 2). Several of the antibodies, including 52M51 and a humanized variant of 52M51, inhibit ligand-induced NOTCH1 signaling (Example 3 and FIGS. 1B and 1C), despite binding NOTCH1 in a region outside of the ligand-binding region. The ability of several of the antibodies to inhibit formation of the NOTCH intracellular domain (ICD) is demonstrated (Examples 3 and 7; FIG. 1D).

### I. DEFINITIONS

To facilitate an understanding of the present invention, a number of terms and phrases are defined below.

The term “antagonist” as used herein refers to any molecule that partially or fully blocks, inhibits, or neutralizes a biological activity of the NOTCH pathway. The term “antagonist” is used herein to include any molecule that partially or fully blocks, inhibits, or neutralizes the expression of a NOTCH receptor. Suitable antagonist molecules specifically include antagonist antibodies or antibody fragments.

The term “antibody” as used herein refers to an immunoglobulin molecule that recognizes and specifically binds a target, such as a protein, polypeptide, peptide, carbohydrate, polynucleotide, lipid, or combinations of the foregoing, through at least one antigen recognition site within the variable region of the immunoglobulin molecule. As used herein, the term encompasses intact polyclonal antibodies, intact monoclonal antibodies, antibody fragments (such as Fab, Fab′, F(ab′)2, and Fv fragments), single chain Fv (scFv) antibodies, multispecific antibodies such as bispecific antibodies generated from at least two intact antibodies, monospecific antibodies, monovalent antibodies, chimeric antibodies, humanized antibodies, human antibodies, fusion proteins comprising an antigen determination portion of an antibody, and any other modified immunoglobulin molecule comprising an antigen recognition site as long as the antibodies exhibit the desired biological activity. An antibody can be any of the five major classes of immunoglobulins: IgA, IgD, IgE, IgG, and IgM, or subclasses (isotypes) thereof (e.g., IgG1, IgG2 IgG3, IgG4, IgA1 and IgA2), based on the identity of their heavy-chain constant domains referred to as alpha, delta, epsilon, gamma, and mu, respectively. The different classes of immunoglobulins have different and well-known subunit structures and three-dimensional configurations. Antibodies can be naked or conjugated to other molecules, including but not limited to, toxins and radioisotopes.

The term “antibody fragment” refers to a portion of an antibody and refers to the antigenic determining variable regions or the antigen-binding regions of an antibody. Examples of antibody fragments include, but are not limited to, Fab, Fab′, F(ab′)2, and Fv fragments, linear antibodies, single chain antibodies, and multispecific antibodies formed from antibody fragments.

The term “variable region” of an antibody refers to the variable region of the antibody light chain or the variable region of the antibody heavy chain, either alone or in combination. The variable regions of the heavy and light chain each consist of four framework regions (FR) connected by three complementarity determining regions (CDRs), also known as “hypervariable regions”. The CDRs in each chain are held together in close proximity by the framework regions and, with the CDRs from the other chain, contribute to the formation of the antigen-binding site of antibodies. There are at least two techniques for determining CDRs: (1) an approach based on cross-species sequence variability (i.e., Kabat et al., 1991, Sequences of Proteins of Immunological Interest 5^{th} ed., National Institutes of Health, Bethesda Md.), and (2) an approach based on crystallographic studies of antigen-antibody complexes (Al-Lazikani et al., 1997, J. Molec. Biol., 273:927-948). In addition, combinations of these two approaches are sometimes used in the art to determine CDRs.

The term “monoclonal antibody” as used herein refers to a homogenous antibody population involved in the highly specific recognition and binding of a single antigenic determinant or epitope. This is in contrast to polyclonal antibodies that typically include a mixture of different antibodies directed against different antigenic determinants. The term “monoclonal antibody” encompasses both intact and full-length monoclonal antibodies as well as antibody fragments (e.g., Fab, Fab′, F(ab′)2, Fv), single chain (scFv) antibodies, fusion proteins comprising an antibody portion, and any other modified immunoglobulin molecule comprising an antigen recognition site. Furthermore, “monoclonal antibody” refers to such antibodies made by any number of techniques, including but not limited to, hybridoma production, phage selection, recombinant expression, and transgenic animals.

The term “humanized antibody” as used herein refers to forms of non-human (e.g., murine) antibodies that are specific immunoglobulin chains, chimeric immunoglobulins, or fragments thereof that contain minimal non-human sequences.

The term “human antibody” as used herein refers to an antibody produced by a human or an antibody having an amino acid sequence corresponding to an antibody produced by a human made using any of the techniques known in the art. This definition of a human antibody includes intact or full-length antibodies, and fragments thereof.

The term “chimeric antibody” as used herein refers to an antibody wherein the amino acid sequence of the immunoglobulin molecule is derived from two or more species. Typically, the variable region of both light and heavy chains corresponds to the variable region of antibodies derived from one species of mammals (e.g., mouse, rat, rabbit, etc.) with the desired specificity, affinity, and/or capability, while the constant regions are homologous to the sequences in antibodies derived from another species (usually human) to avoid eliciting an immune response in that species.

The terms “epitope” and “antigenic determinant” are used interchangeably herein and refer to that portion of an antigen capable of being recognized and specifically bound by a particular antibody. When the antigen is a polypeptide, epitopes can be formed both from contiguous amino acids and noncontiguous amino acids juxtaposed by tertiary folding of a protein. Epitopes formed from contiguous amino acids (also referred to as linear epitopes) are typically retained upon protein denaturing, whereas epitopes formed by tertiary folding (also referred to as conformational epitopes) are typically lost upon protein denaturing. An epitope typically includes at least 3, and more usually, at least 5 or 8-10 amino acids in a unique spatial conformation.

The terms “selectively binds” or “specifically binds” mean that an antibody reacts or associates more frequently, more rapidly, with greater duration, with greater affinity, or with some combination of the above to the epitope, protein, or target molecule than with alternative substances, including related and unrelated proteins. In certain embodiments “specifically binds” means, for instance, that an antibody binds a protein with a K_{D} of about 0.1 mM or less, but more usually less than about 1 uM. In certain embodiments, “specifically binds” means that an antibody binds a target at times with a K_{D} of at least about 0.1 μM or less and at other times at least about 0.01 μM or less. Because of the sequence identity between homologous proteins in different species, specific binding can include an antibody that recognizes a protein (e.g., a NOTCH receptor) in more than one species. Likewise, because of homology within certain regions of polypeptide sequences of different proteins, specific binding can include an antibody that recognizes more than one protein. It is understood that, in certain embodiments, an antibody that specifically binds a first target may or may not specifically bind a second target. As such, “specific binding” does not necessarily require (although it can include) exclusive binding, i.e. binding a single target. Thus, an antibody may, in certain embodiments, specifically bind more than one target. In certain embodiments, the multiple targets may be bound by the same antigen-binding site on the antibody. For example, an antibody may, in certain instances, comprise two identical antigen-binding sites, each of which specifically binds the same epitope on two or more proteins. In certain alternative embodiments, an antibody may be bispecific and comprise at least two antigen-binding sites with differing specificities. By way of non-limiting example, a bispecific antibody may comprise one antigen-binding site that recognizes an epitope on one protein and further comprises a second, different antigen-binding site that recognizes a different epitope on a second protein. Generally, but not necessarily, reference to binding means specific binding.

The terms “polypeptide” and “peptide” and “protein” are used interchangeably herein and refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids), as well as other modifications known in the art. It is understood that, because the polypeptides of this invention are based upon antibodies, in certain embodiments, the polypeptides can occur as single chains or as associated chains.

The terms “polynucleotide” and “nucleic acid” are used interchangeably herein and refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase.

The terms “identical” or percent “identity” in the context of two or more nucleic acids or polypeptides, refer to two or more sequences or subsequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity may be measured using sequence comparison software or algorithms or by visual inspection. Various algorithms and software that may be used to obtain alignments of amino acid or nucleotide sequences are well-known in the art. These include but are not limited to, BLAST, ALIGN, Megalign, BestFit, GCG Wisconsin Package, and variants thereof. In some embodiments, two nucleic acids or polypeptides of the invention are substantially identical, meaning they have at least 70%, at least 75%, at least 80%, at least 85%, at least 90%, and in some embodiments at least 95%, 96%, 97%, 98%, 99% nucleotide or amino acid residue identity, when compared and aligned for maximum correspondence, as measured using a sequence comparison algorithm or by visual inspection. In some embodiments, identity exists over a region of the sequences that is at least about 10, at least about 20, at least about 40-60 residues in length or any integral value therebetween. In some embodiments, identity exists over a longer region than 60-80 residues, such as at least about 90-100 residues, and in some embodiments the sequences are substantially identical over the full length of the sequences being compared, such as the coding region of a nucleotide sequence.

A “conservative amino acid substitution” is one in which one amino acid residue is replaced with another amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), non-polar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). For example, substitution of a phenylalanine for a tyrosine is a conservative substitution. Preferably, conservative substitutions in the sequences of the antibodies of the invention do not abrogate the binding of the antibody containing the amino acid sequence, to the antigen(s), i.e., the one or more NOTCH proteins to which the antibody binds. Methods of identifying nucleotide and amino acid conservative substitutions which do not eliminate antigen binding are well-known in the art.

The term “vector” as used herein means a construct, which is capable of delivering, and usually expressing, one or more gene(s) or sequence(s) of interest in a host cell. Examples of vectors include, but are not limited to, viral vectors, naked DNA or RNA expression vectors, plasmid, cosmid or phage vectors, DNA or RNA expression vectors associated with cationic condensing agents, and DNA or RNA expression vectors encapsulated in liposomes.

A polypeptide, antibody, polynucleotide, vector, cell, or composition which is “isolated” is a polypeptide, antibody, polynucleotide, vector, cell, or composition which is in a form not found in nature. Isolated polypeptides, antibodies, polynucleotides, vectors, cells, or compositions include those which have been purified to a degree that they are no longer in a form in which they are found in nature. In some embodiments, a polypeptide, antibody, polynucleotide, vector, cell, or composition which is isolated is substantially pure.

The term “substantially pure” as used herein refers to material which is at least 50% pure (i.e., free from contaminants), at least 90% pure, at least 95% pure, at least 98% pure, or at least 99% pure.

The terms “cancer” and “cancerous” as used herein refer to or describe the physiological condition in mammals in which a population of cells are characterized by unregulated cell growth. Examples of cancer include, but are not limited to, carcinoma, blastoma, sarcoma, and hematologic cancers such as lymphoma and leukemia. Hematologic cancers include, but are not limited to, chronic lymphocytic leukemia (CLL), hairy cell leukemia, chronic myelogenous leukemia (CML), non-Hodgkin lymphoma, diffuse large B-cell lymphoma (DLBCL), mantle cell lymphoma (MCL), and cutaneous T-cell lymphoma (CTCL). Cutaneous T-cell lymphomas include mycosis fungoides (e.g., transformed mycosis fungoides) and Sézary Syndrome. Hematologic cancers also include NK-cell leukemia, splenic marginal zone lymphoma, and follicular lymphoma.

The terms “tumor” and “neoplasm” as used herein refer to any mass of tissue that results from excessive cell growth or proliferation, either benign (non-cancerous) or malignant (cancerous) including pre-cancerous lesions.

The terms “proliferative disorder” and “proliferative disease” refer to disorders associated with abnormal cell proliferation such as cancer.

The term “metastasis” as used herein refers to the process by which a cancer spreads or transfers from the site of origin to other regions of the body with the development of a similar cancerous lesion at the new location. A “metastatic” or “metastasizing” cell may be one that loses adhesive contacts with neighboring cells and migrates via the bloodstream or lymph from the primary site of disease to invade neighboring body structures.

The terms “cancer stem cell” and “CSC” and “tumor stem cell” are used interchangeably herein and refer to cells from a cancer that: (1) have extensive proliferative capacity; 2) are capable of asymmetric cell division to generate one or more kinds of differentiated progeny with reduced proliferative or developmental potential; and (3) are capable of symmetric cell divisions for self-renewal or self-maintenance. These properties confer on the cancer stem cells the ability to form or establish a tumor or cancer upon serial transplantation into an immunocompromised host (e.g., a mouse) compared to the majority of tumor cells that fail to form tumors. Cancer stem cells undergo self-renewal versus differentiation in a chaotic manner to form tumors with abnormal cell types that can change over time as mutations occur. “Cancer stem cell” as used herein may comprise leukemia-initiating cells.

The terms “cancer cell” and “tumor cell” refer to the total population of cells derived from a cancer or tumor or pre-cancerous lesion, including both non-tumorigenic cells, which comprise the bulk of the cancer cell population, and tumorigenic stem cells (cancer stem cells). As used herein, the terms “cancer cell” or “tumor cell” will be modified by the term “non-tumorigenic” when referring solely to those cells lacking the capacity to renew and differentiate to distinguish those tumor cells from cancer stem cells.

The term “tumorigenic” as used herein refers to the functional features of a cancer stem cell including the properties of self-renewal (giving rise to additional tumorigenic cancer stem cells) and proliferation to generate all other tumor cells (giving rise to differentiated and thus non-tumorigenic tumor cells).

The term “tumorigenicity” as used herein of a tumor refers to the ability of a random sample of cells from the tumor to form palpable tumors upon serial transplantation into host animals hosts (e.g., immunocompromised mice).

The term “subject” refers to any animal (e.g., a mammal), including, but not limited to, humans, non-human primates, canines, felines, rodents, and the like, which is to be the recipient of a particular treatment. Typically, the terms “subject” and “patient” are used interchangeably herein in reference to a human subject.

The term “pharmaceutically acceptable” refers to a compound approved or approvable by a regulatory agency of the Federal government or a state government or listed in the U.S. Pharmacopeia or other generally recognized pharmacopeia for use in animals, including humans.

The terms “pharmaceutically acceptable excipient, carrier or adjuvant” or “acceptable pharmaceutical carrier” refer to an excipient, carrier or adjuvant that can be administered to a subject, together with at least one NOTCH1-binding antibody of the present disclosure, and which does not destroy the pharmacological activity thereof and is nontoxic when administered in doses sufficient to deliver a therapeutic effect.

The terms “effective amount” or “therapeutically effective amount” or “therapeutic effect” refer to an amount of an antibody, polypeptide, polynucleotide, small organic molecule, or other drug effective to “treat” a disease or disorder in a subject or mammal. In the case of cancer, the therapeutically effective amount of a drug (e.g., an antibody) has a therapeutic effect and as such can reduce the number of cancer cells; decrease tumorigenicity, tumorigenic frequency or tumorigenic capacity; reduce the number or frequency of cancer stem cells; reduce the tumor size; reduce the cancer cell population; inhibit or stop cancer cell infiltration into peripheral organs including, for example, the spread of cancer into soft tissue and bone; inhibit and stop tumor or cancer cell metastasis; inhibit and stop tumor or cancer cell growth; relieve to some extent one or more of the symptoms associated with the cancer; reduce morbidity and mortality; improve quality of life; or a combination of such effects. To the extent the agent, for example an antibody, prevents growth and/or kills existing cancer cells, it can be referred to as cytostatic and/or cytotoxic.

The terms “treating” or “treatment” or “to treat” or “alleviating” or “to alleviate” refer to both 1) therapeutic measures that cure, slow down, lessen symptoms of, and/or halt progression of a diagnosed pathologic condition or disorder and 2) prophylactic or preventative measures that prevent or slow the development of a targeted pathologic condition or disorder. Thus those in need of treatment include those already with the disorder; those prone to have the disorder; and those in whom the disorder is to be prevented. In some embodiments, a subject is successfully “treated” according to the methods of the present invention if the patient shows one or more of the following: a reduction in the number of or complete absence of cancer cells; a reduction in the tumor size; inhibition of or an absence of cancer cell infiltration into peripheral organs including the spread of cancer cells into soft tissue and bone; inhibition of or an absence of tumor or cancer cell metastasis; inhibition or an absence of cancer growth; relief of one or more symptoms associated with the specific cancer; reduced morbidity and mortality; improvement in quality of life; reduction in tumorigenicity; reduction in the number or frequency of cancer stem cells; or some combination of effects.

As used in the present disclosure and claims, the singular forms “a”, “an”, and “the” include plural forms unless the context clearly dictates otherwise.

It is understood that wherever embodiments are described herein with the language “comprising” otherwise analogous embodiments described in terms of “consisting of” and/or “consisting essentially of” are also provided.

The term “and/or” as used in a phrase such as “A and/or B” herein is intended to include: both A and B; A or B; A (alone); and B alone. Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

### II. NOTCH1-BINDING ANTIBODIES

The present invention provides methods of using antibodies that bind (e.g., specifically bind) to human NOTCH1. In certain embodiments, the antibodies bind the extracellular domain of NOTCH1. In some embodiments, the antibody is an antagonist of NOTCH1 or inhibits NOTCH1 signaling and/or activation of NOTCH1. In certain embodiments, the antibody is a monoclonal antibody.

The present invention also provides antibodies that specifically bind to a non-ligand binding membrane proximal region of the extracellular domain of human NOTCH1, compositions comprising those antibodies and methods for using those antibodies to treat hematologic cancers. In particular, in certain embodiments, the present invention provides antibodies, including antagonists, that bind NOTCH1 and methods of using the agents or antagonists to inhibit cancer growth and treat cancer in human patients. In certain embodiments, the antagonists are antibodies that specifically bind to a non-ligand binding region of the extracellular domain of human NOTCH1.

In some embodiments, the antibody binds a region of human NOTCH1 comprising about amino acid 1427 to about amino acid 1732. In some embodiments, the antibody binds a region comprising SEQ ID NO:2. In some embodiments, the antibody specifically binds a region within SEQ ID NO:2. In some embodiments, the antibody specifically binds an epitope within a region comprising SEQ ID NO:2. In certain embodiments, the antibody that binds NOTCH1 also specifically binds a non-ligand binding membrane proximal region of the extracellular domain of at least one additional NOTCH receptor. In some embodiments, the at least one additional NOTCH receptor is NOTCH2. In some embodiments, the at least one additional NOTCH receptor is NOTCH3. In some embodiments, the at least one additional NOTCH receptor is NOTCH4.

In certain embodiments, the antibody is an IgG antibody. In some embodiments, the antibody is an IgG1 antibody. In some embodiments, the antibody is an IgG2 antibody. In certain embodiments, the antibody is a monoclonal antibody. In certain embodiments, the antibody is a humanized antibody. In certain embodiments, the antibody is a human antibody. In certain embodiments, the antibody is an antibody fragment comprising an antigen-binding site. In some embodiments, the antibody is monovalent, monospecific, bivalent, bispecific, or multispecific. In some embodiments, the antibody is conjugated to a cytotoxic moiety. In some embodiments, the antibody is isolated. In some embodiments, the antibody is substantially pure.

In certain embodiments, the NOTCH1-binding antibody binds a non-ligand binding membrane proximal region of the extracellular domain of human NOTCH1 with a dissociation constant (K_{D}) of about 1 μM or less, about 100 nM or less, about 40 nM or less, about 20 nM or less, about 10 nM or less or about 1 nM or less. In certain embodiments, the NOTCH1-binding antibody binds human NOTCH1 with a K_{D} of about 40 nM or less, about 20 nM or less, about 10 nM or less, or about 1 nM or less. In some embodiments, the dissociation constant of the antibody to NOTCH1 is a dissociation constant determined using a NOTCH1 fusion protein comprising a proximal region of the NOTCH1 extracellular domain immobilized on a Biacore chip.

The NOTCH1-binding antibodies of the present invention can be assayed for specific binding by any method known in the art. The immunoassays which can be used include, but are not limited to, competitive and non-competitive assay systems using techniques such as Biacore analysis, FACS analysis, immunofluorescence, immunocytochemistry, Western blot analysis, radioimmunoassay, ELISA, “sandwich” immunoassay, immunoprecipitation assay, precipitation reaction, gel diffusion precipitin reaction, immunodiffusion assay, agglutination assay, complement-fixation assay, immunoradiometric assay, fluorescent immunoassay, and protein A immunoassay. Such assays are routine and well-known in the art (see, e.g., Ausubel et al., Eds., 1994, Current Protocols in Molecular Biology, Vol. 1, John Wiley & Sons, Inc., New York).

In some embodiments, the specific binding of a NOTCH1-binding antibody to human NOTCH1 may be determined using ELISA. In some embodiments, an ELISA assay comprises preparing NOTCH1 antigen, coating wells of a 96-well microtiter plate with antigen, adding to the wells the NOTCH1-binding antibody conjugated to a detectable compound such as an enzymatic substrate (e.g., horseradish peroxidase or alkaline phosphatase), incubating for a period of time and detecting the presence of the binding antibody. In some embodiments, the antibody is not conjugated to a detectable compound, but instead a second conjugated antibody that recognizes the NOTCH1-binding antibody is added to the well. In some embodiments, instead of coating the well with the NOTCH1 antigen, the antibody can be coated to the well, antigen is added to the coated well and then a second antibody conjugated to a detectable compound is added. One of skill in the art would be knowledgeable as to the parameters that can be modified and/or optimized to increase the signal detected, as well as other variations of ELISAs that can be used (see e.g., Ausubel et al., Eds., 1994, Current Protocols in Molecular Biology, Vol. 1, John Wiley & Sons, Inc., New York at 11.2.1).

In another example, the specific binding of an antibody to human NOTCH1 may be determined using FACS. A FACS screening assay may comprise generating a cDNA construct that expresses an antigen as a fusion protein, transfecting the construct into cells, expressing the antigen on the surface of the cells, mixing the NOTCH-binding antibody with the transfected cells, and incubating for a period of time. The cells bound by the NOTCH-binding antibody may be identified by using a secondary antibody conjugated to a detectable compound (e.g., PE-conjugated anti-Fc antibody) and a flow cytometer. One of skill in the art would be knowledgeable as to the parameters that can be modified to optimize the signal detected as well as other variations of FACS that may enhance screening (e.g., screening for blocking antibodies).

The binding affinity of an antibody to NOTCH1 and the on-off rate of an antibody-antigen interaction can be determined by competitive binding assays. In some embodiments, a competitive binding assay is a radioimmunoassay comprising the incubation of labeled antigen (e.g., ³H- or ¹²⁵I-labeled antigen), or fragment or variant thereof, with the antibody of interest in the presence of increasing amounts of unlabeled antigen followed by the detection of the antibody bound to the labeled antigen. The affinity of the antibody for the antigen and the on-off rates can be determined from the data by Scatchard plot analysis. In some embodiments, Biacore kinetic analysis is used to determine the binding affinities and on-off rates of antibodies that bind NOTCH (e.g., human NOTCH1, human NOTCH2, human NOTCH3, human NOTCH 4, and/or mouse NOTCH). Biacore kinetic analysis comprises analyzing the binding and dissociation of antibodies from antigens (e.g., NOTCH1 proteins) that have been immobilized on the surface of a Biacore chip. In some embodiments, Biacore kinetic analyses can be used to study binding of different antibodies in qualitative epitope competition binding assays.

In certain embodiments, the invention provides an antibody that specifically binds a non-ligand binding membrane proximal region of the extracellular domain of human NOTCH1, wherein the antibody comprises one, two, three, four, five, and/or six of the CDRs of antibody 52M51 (see Table 1). In some embodiments, the antibody comprises one or more of the CDRs of 52M51, two or more of the CDRs of 52M51, three or more of the CDRs of 52M51, four or more of the CDRs of 52M51, five or more of the CDRs of 52M51, or all six of the CDRs or 52M51. In some embodiments, the antibody comprises CDRs with up to four (i.e., 0, 1, 2, 3, or 4) amino acid substitutions per CDR. In certain embodiments, the heavy chain CDR(s) are contained within a heavy chain variable region. In certain embodiments, the light chain CDR(s) are contained within a light chain variable region.

TABLE 1

| | 52M51 |
|---|---|
| HCCDR1 | RGYWIE |
| | (SEQ ID NO: 15), |
| HCCDR2 | QILPGTGRTNYNEKFKG |
| | (SEQ ID NO: 16) |
| HCCDR3 | FDGNYGYYAMDY |
| | (SEQ ID NO: 17) |
| LCCDR1 | RSSTGAVTTSNYAN |
| | (SEQ ID NO: 18) |
| LCCDR2 | GTNNRAP |
| | (SEQ ID NO: 19) |
| LCCDR3 | ALWYSNHWVFGGGTKL |
| | (SEQ ID NO: 20) |

In certain embodiments, the antibody comprises (a) a heavy chain CDR1 comprising RGYWIE (SEQ ID NO:15), a heavy chain CDR2 comprising QILPGTGRTNYNEKFKG (SEQ ID NO: 16), and/or a heavy chain CDR3 comprising FDGNYGYYAMDY (SEQ ID NO: 17); and/or (b) a light chain CDR1 comprising RSSTGAVTTSNYAN (SEQ ID NO:18), a light chain CDR2 comprising GTNNRAP (SEQ ID NO:19), and/or a light chain CDR3 comprising ALWYSNHWVFGGGTKL (SEQ ID NO:20). In some embodiments, the antibody comprises a heavy chain variable region comprising: (a) a heavy chain CDR1 comprising RGYWIE (SEQ ID NO:15), or a variant thereof comprising 1, 2, 3, or 4 amino acid substitutions; (b) a heavy chain CDR2 comprising QILPGTGRTNYNEKFKG (SEQ ID NO: 16), or a variant thereof comprising 1, 2, 3, or 4 amino acid substitutions; and/or (c) a heavy chain CDR3 comprising FDGNYGYYAMDY (SEQ ID NO:17), or a variant thereof comprising 1, 2, 3, or 4 amino acid substitutions. In other embodiments, the antibody comprises (or further comprises) a light chain variable region comprising: (a) a light chain CDR1 comprising RSSTGAVTTSNYAN (SEQ ID NO:18), or a variant thereof comprising 1, 2, 3, or 4 amino acid substitutions; (b) a light chain CDR2 comprising GTNNRAP (SEQ ID NO:19) or a variant thereof comprising 1, 2, 3, or 4 amino acid substitutions; and/or (c) a light chain CDR3 comprising ALWYSNHWVFGGGTKL (SEQ ID NO:20), or a variant thereof comprising 1, 2, 3, or 4 amino acid substitutions. In some embodiments, the amino acid substitutions are conservative amino acid substitutions.

In some embodiments, the antibody comprises a heavy chain variable region having at least about 90% sequence identity to SEQ ID NO: 14, and/or a light chain variable region having at least about 90% sequence identity to SEQ ID NO:8. In some embodiments, the antibody comprises a heavy chain variable region having at least about 95%, at least about 97%, or at least about 99% sequence identity to SEQ ID NO: 14, and/or a light chain variable region having at least about 95%, at least about 97%, or at least about 99% sequence identity to SEQ ID NO:8. In some embodiments, the antibody comprises a heavy chain variable region having at least about 95% sequence identity to SEQ ID NO: 14, and/or a light chain variable region having at least about 95% sequence identity to SEQ ID NO:8. In some embodiments, the antibody comprises a heavy chain variable region comprising SEQ ID NO: 14, and/or a light chain variable region comprising SEQ ID NO:8. In some embodiments, the antibody comprises a heavy chain variable region comprising SEQ ID NO:14, and a light chain variable region comprising SEQ ID NO:8. In some embodiments, the antibody is a monoclonal antibody or antibody fragment.

In some embodiments, the antibody comprises a heavy chain variable region having at least about 90% sequence identity to SEQ ID NO:24, and/or a light chain variable region having at least about 90% sequence identity to SEQ ID NO:28. In some embodiments, the antibody comprises a heavy chain variable region having at least about 95%, at least about 97%, or at least about 99% sequence identity to SEQ ID NO:24, and/or a light chain variable region having at least about 95%, at least about 97%, or at least about 99% sequence identity to SEQ ID NO:28. In some embodiments, the antibody comprises a heavy chain variable region having at least about 95% sequence identity to SEQ ID NO:24, and/or a light chain variable region having at least about 95% sequence identity to SEQ ID NO:28. In some embodiments, the antibody comprises a heavy chain variable region comprising SEQ ID NO:24, and/or a light chain variable region comprising SEQ ID NO:28. In some embodiments, the antibody comprises a heavy chain variable region comprising SEQ ID NO:24, and a light chain variable region comprising SEQ ID NO:28. In some embodiments, the antibody is a monoclonal antibody or antibody fragment.

In some embodiments, the antibody comprises a heavy chain variable region having at least about 90% sequence identity to SEQ ID NO:24, and/or a light chain variable region having at least about 90% sequence identity to SEQ ID NO:32. In some embodiments, the antibody comprises a heavy chain variable region having at least about 95%, at least about 97%, or at least about 99% sequence identity to SEQ ID NO:24, and/or a light chain variable region having at least about 95%, at least about 97%, or at least about 99% sequence identity to SEQ ID NO:32. In some embodiments, the antibody comprises a heavy chain variable region having at least about 95% sequence identity to SEQ ID NO:24, and/or a light chain variable region having at least about 95% sequence identity to SEQ ID NO:32. In some embodiments, the antibody comprises a heavy chain variable region comprising SEQ ID NO:24, and/or a light chain variable region comprising SEQ ID NO:32. In some embodiments, the antibody comprises a heavy chain variable region comprising SEQ ID NO:24, and a light chain variable region comprising SEQ ID NO:32. In some embodiments, the antibody is a monoclonal antibody or antibody fragment.

In some embodiments, the NOTCH1-binding antibody is an antibody, 52M51, produced by the hybridoma cell line deposited with the ATCC under the conditions of the Budapest Treaty on Aug. 7, 2008 and assigned number PTA-9405. In some embodiments, the antibody is a humanized version of 52M51. In some embodiments, the antibody is a humanized version of 52M51, “52M51-H4L3”, as encoded by the DNA deposited with the ATCC under the conditions of the Budapest Treaty on Oct. 15, 2008 and assigned number PTA-9549. In some embodiments, the antibody is a humanized version of 52M51, “52M51-H4L4”. In some embodiments, the invention provides an antibody that binds the same epitope as the epitope to which antibody 52M51 binds. In other embodiments, the invention provides an antibody that competes with any of the antibodies described in the aforementioned embodiments and/or aspects, as well as other aspects/embodiments described elsewhere herein, for specific binding to a non-ligand binding membrane proximal region of the extracellular domain of human NOTCH1.

In certain embodiments, the antibody binds NOTCH1 and modulates NOTCH1 activity or signaling. In some embodiments, the antibody is an antagonist and modulates NOTCH1 activity or signaling. In certain embodiments, the antibody is an antagonist of NOTCH1 and inhibits NOTCH1 signaling. In certain embodiments, the antibody inhibits at least about 10%, at least about 20%, at least about 30%, at least about 50%, at least about 75%, at least about 90%, or about 100% of NOTCH1 signaling or activity. In some embodiments, the antibody inhibits activity and/or signaling of a constitutively activated NOTCH1. In some embodiments, the constitutively activated NOTCH1 is expressed in a hematologic cancer. In certain embodiments, the constitutively activated NOTCH1 is expressed in a chronic lymphocytic leukemia.

In certain embodiments, the antibody inhibits NOTCH activation. It is understood that a NOTCH1-binding antibody that inhibits NOTCH activation may, in certain embodiments, inhibit activation of one or more NOTCHs, but not necessarily inhibit activation of all NOTCHs. In certain alternative embodiments, activation of all human NOTCHs may be inhibited. In certain embodiments, activation of NOTCH1 and one or more additional NOTCHs selected from the group consisting of NOTCH2, NOTCH3, and NOTCH4 is inhibited. In certain embodiments, the inhibition of NOTCH activation by a NOTCH1-binding antibody is a reduction in the level of NOTCH1 activation of at least about 10%, at least about 25%, at least about 50%, at least about 75%, at least about 90%, or at least about 95%.

In vivo and in vitro assays for determining whether an antibody inhibits NOTCH activation are known in the art. In some embodiments, a cell-based, luciferase reporter assay utilizing a TCF/Luc reporter vector containing multiple copies of the TCF-binding domain upstream of a firefly luciferase reporter gene may be used to measure NOTCH signaling levels in vitro. In other embodiments, a cell-based, luciferase reporter assay utilizing a CBF/Luc reporter vector containing multiple copies of the CBF-binding domain upstream of a firefly luciferase reporter gene may be used. The level of NOTCH activation induced by a NOTCH ligand in the presence of a NOTCH1-binding antibody is compared to the level of NOTCH activation induced by a NOTCH ligand in the absence of an antibody. Non-limiting, specific examples of the use of such luciferase reporter assays to assess inhibition of NOTCH activation are provided in Example 3 and FIGS. 1B and 1C.

In certain embodiments, the antibodies have one or more of the following effects: inhibit proliferation of cancer cells, inhibit cancer cell growth, prevent or reduce metastasis of cancer cells, reduce the frequency of cancer stem cells in a tumor or cancer, trigger cell death of cancer cells (e.g., by apoptosis), reduce the tumorigenicity of cancer cells by reducing the frequency of cancer stem cells in the cancer cell population, differentiate tumorigenic cells to a non-tumorigenic state, or increase survival of a patient.

In certain embodiments, the antibodies are capable of inhibiting cancer cell growth. In certain embodiments, the antibodies are capable of inhibiting growth of cancer cells in vitro (e.g., contacting cancer cells with an antibody in vitro). In certain embodiments, the antibodies are capable of inhibiting cancer growth in vivo (e.g., in a xenograft mouse model and/or in a human having cancer).

In certain embodiments, the antibodies are capable of reducing the tumorigenicity of a hematologic cancer. In certain embodiments, the antibodies are capable of reducing the tumorigenicity of a hematologic cancer comprising cancer stem cells in an animal model, such as a mouse xenograft model. In certain embodiments, the antibodies are capable of reducing the tumorigenicity of a hematologic cancer comprising cancer stem cells in an animal model, such as a mouse xenograft model. In some embodiments, the antibody is capable of reducing the tumorigenicity of a hematologic cancer by reducing the frequency of cancer stem cells in the cancer. In certain embodiments, the number or frequency of cancer stem cells in a cancer is reduced by at least about two-fold, about three-fold, about five-fold, about ten-fold, about 50-fold, about 100-fold, or about 1000-fold. In certain embodiments, the reduction in the frequency of cancer stem cells is determined by a limiting dilution assay (LDA) using an animal model. Examples and guidance regarding the use of limiting dilution assays to determine a reduction in the number or frequency of cancer stem cells in a tumor can be found, e.g., in International Pub. No. WO 2008/042236 and U.S. Patent Pub. Nos. 2008/0064049 and 2008/0178305.

In certain embodiments, the antibody has a circulating half-life in a subject or mammal (e.g., mice, rats, cynomolgus monkeys, or humans) of at least about 5 hours, at least about 10 hours, at least about 24 hours, at least about 3 days, at least about 1 week, or at least about 2 weeks. In certain embodiments, the antibody is an IgG (e.g., IgG1 or IgG2) antibody that has a circulating half-life in a subject or mammal (e.g., mice, rats, cynomolgus monkeys, or humans) of at least about 5 hours, at least about 10 hours, at least about 24 hours, at least about 3 days, at least about 1 week, or at least about 2 weeks. Methods of increasing the half-life of antibodies are known in the art. In some embodiments, known methods of increasing the circulating half-life of IgG antibodies include the introduction of mutations in the Fc region which increase the pH-dependent binding of the antibody to the neonatal Fc receptor (FcRn) at pH 6.0 (see e.g., U.S. Patent Pub. Nos. 2005/0276799; 2007/0148164; and 2007/0122403). Known methods of increasing the circulating half-life of antibody fragments lacking the Fc region include, but are not limited to, techniques such as PEGylation.

In some embodiments, the NOTCH1-binding antibodies are polyclonal antibodies. Polyclonal antibodies can be prepared by any known method. In some embodiments, polyclonal antibodies are raised by immunizing an animal (e.g. a rabbit, rat, mouse, goat, or donkey) by multiple subcutaneous or intraperitoneal injections of the relevant antigen (e.g., a purified peptide fragment, full-length recombinant protein, or fusion protein). The antigen can be optionally conjugated to a carrier such as keyhole limpet hemocyanin (KLH) or serum albumin. The antigen (with or without a carrier protein) is diluted in sterile saline and usually combined with an adjuvant (e.g., Complete or Incomplete Freund's Adjuvant) to form a stable emulsion. After a sufficient period of time, polyclonal antibodies are recovered from blood, ascites and the like, of the immunized animal. The polyclonal antibodies can be purified from serum or ascites according to standard methods in the art including, but not limited to, affinity chromatography, ion-exchange chromatography, gel electrophoresis, and dialysis.

In some embodiments, the NOTCH1-binding antibodies are monoclonal antibodies. Monoclonal antibodies can be prepared using hybridoma methods known to one of skill in the art (see e.g., Kohler and Milstein, 1975, Nature 256:495-497). In some embodiments, using the hybridoma method, a mouse, hamster, or other appropriate host animal, is immunized as described above to elicit from lymphocytes the production of antibodies that will specifically bind to the immunizing antigen. In some embodiments, lymphocytes can be immunized in vitro. In some embodiments, the immunizing antigen can be a human protein or a portion thereof. In some embodiments, the immunizing antigen can be a mouse protein or a portion thereof.

Following immunization, lymphocytes are isolated and fused with a suitable myeloma cell line using, for example, polyethylene glycol, to form hybridoma cells that can then be selected away from unfused lymphocytes and myeloma cells. Hybridomas that produce monoclonal antibodies directed specifically against a chosen antigen may be identified by a variety of methods including, but not limited to, immunoprecipitation, immunoblotting, and in vitro binding assay (e.g., flow cytometry, enzyme-linked immunosorbent assay (ELISA), and radioimmunoassay (RIA)). The hybridomas can be propagated either in in vitro culture using standard methods (Goding, Monoclonal Antibodies: Principles and Practice, Academic Press, 1986) or in in vivo as ascites tumors in an animal. The monoclonal antibodies can be purified from the culture medium or ascites fluid according to standard methods in the art including, but not limited to, affinity chromatography, ion-exchange chromatography, gel electrophoresis, and dialysis.

In certain embodiments, monoclonal antibodies can be made using recombinant DNA techniques as known to one skilled in the art (see e.g., U.S. Pat. No. 4,816,567). The polynucleotides encoding a monoclonal antibody are isolated from mature B-cells or hybridoma cells, such as by RT-PCR using oligonucleotide primers that specifically amplify the genes encoding the heavy and light chains of the antibody, and their sequence is determined using conventional techniques. The isolated polynucleotides encoding the heavy and light chains are then cloned into suitable expression vectors which produce the monoclonal antibodies when transfected into host cells such as E. coli, simian COS cells, Chinese hamster ovary (CHO) cells, or myeloma cells that do not otherwise produce immunoglobulin protein. In other embodiments, recombinant monoclonal antibodies, or fragments thereof, can be isolated from phage display libraries expressing CDRs of the desired species (see e.g., McCafferty et al., 1990, Nature, 348:552-554; Clackson et al., 1991, Nature, 352:624-628; and Marks et al., 1991, J. Mol. Biol., 222:581-597).

The polynucleotide(s) encoding a monoclonal antibody can further be modified using recombinant DNA technology to generate alternative antibodies. In some embodiments, the constant domains of the light and heavy chains of, for example, a mouse monoclonal antibody can be substituted 1) for those regions of, for example, a human antibody to generate a chimeric antibody or 2) for a non-immunoglobulin polypeptide to generate a fusion antibody. In other embodiments, the constant regions are truncated or removed to generate the desired antibody fragment of a monoclonal antibody. Site-directed or high-density mutagenesis of the variable region can be used to optimize specificity, affinity, and/or other biological characteristics of a monoclonal antibody. In some embodiments, site-directed mutagenesis of the CDRs can be used to optimize specificity, affinity, and/or other biological characteristics of a monoclonal antibody.

In some embodiments, the NOTCH1-binding antibody is a humanized antibody. Typically, humanized antibodies are human immunoglobulins in which residues from the CDRs are replaced by residues from a CDR of a non-human species (e.g., mouse, rat, rabbit, or hamster) that have the desired specificity, affinity, and/or capability using methods known to one skilled in the art. In some embodiments, the Fv framework region residues of a human immunoglobulin are replaced with the corresponding residues in an antibody from a non-human species that has the desired specificity, affinity, and/or capability. In some embodiments, the humanized antibody can be further modified by the substitution of additional residues either in the Fv framework region and/or within the replaced non-human residues to refine and optimize antibody specificity, affinity, and/or capability. In general, the humanized antibody will comprise substantially all of at least one, and typically two or three, variable domains containing all, or substantially all, of the CDR regions that correspond to the non-human immunoglobulin whereas all, or substantially all, of the framework regions are those of a human immunoglobulin consensus sequence. In some embodiments, the humanized antibody can also comprise at least a portion of an immunoglobulin constant region or domain (Fc), typically that of a human immunoglobulin. In certain embodiments, such humanized antibodies are used therapeutically because they may reduce antigenicity and HAMA (human anti-mouse antibody) responses when administered to a human subject. One skilled in the art would be able to obtain a functional humanized antibody with reduced immunogenicity following known techniques (see e.g., U.S. Pat. Nos. 5,225,539; 5,585,089; 5,693,761; and 5,693,762).

In certain embodiments, the NOTCH1-binding antibody is a human antibody. Human antibodies can be directly prepared using various techniques known in the art. In some embodiments, immortalized human B lymphocytes immunized in vitro or isolated from an immunized individual that produces an antibody directed against a target antigen can be generated (see, e.g., Cole et al., Monoclonal Antibodies and Cancer Therapy, Alan R. Liss, p. 77 (1985); Boemer et al., 1991, J. Immunol., 147 (1):86-95; and U.S. Pat. Nos. 5,750,373; 5,567,610 and 5,229,275). In some embodiments, the human antibody can be selected from a phage library, where that phage library expresses human antibodies (Vaughan et al., 1996, Nature Biotechnology, 14:309-314; Sheets et al., 1998, PNAS, 95:6157-6162; Hoogenboom and Winter, 1991, J. Mol. Biol., 227:381; Marks et al., 1991, J. Mol. Biol., 222:581). Alternatively, phage display technology can be used to produce human antibodies and antibody fragments in vitro, from immunoglobulin variable (V) domain gene repertoires from unimmunized donors. Techniques for the generation and use of antibody phage libraries are also described in U.S. Pat. Nos. 5,969,108; 6,172,197; 5,885,793; 6,521,404; 6,544,731; 6,555,313; 6,582,915; 6,593,081; 6,300,064; 6,653,068; 6,706,484; and 7,264,963; and Rothe et al., 2008, J. Mol. Bio., 376:1182-1200. Affinity maturation strategies, such as chain shuffling (Marks et al., 1992, Bio/Technology, 10:779-783), are known in the art and may be employed to generate high affinity human antibodies.

In some embodiments, human antibodies can be made in transgenic mice containing human immunoglobulin loci that are capable, upon immunization, of producing the full repertoire of human antibodies in the absence of endogenous immunoglobulin production. This approach is described in U.S. Pat. Nos. 5,545,807; 5,545,806; 5,569,825; 5,625,126; 5,633,425; and 5,661,016.

In some embodiments, the NOTCH1-binding antibody is a bispecific antibody. Bispecific antibodies are capable of specifically recognizing and binding at least two different epitopes. The different epitopes can either be within the same molecule or on different molecules. In some embodiments, the bispecific antibodies are monoclonal human or humanized antibodies. In some embodiments, the antibodies can specifically recognize and bind a first antigen target, (e.g., NOTCH1) as well as a second antigen target, such as an effector molecule on a leukocyte (e.g., CD2, CD3, CD28, or B7) or a Fc receptor (e.g., CD64, CD32, or CD16) so as to focus cellular defense mechanisms to the cell expressing the first antigen target. In some embodiments, the antibodies can be used to direct cytotoxic agents to cells which express a particular target antigen, such as NOTCH1. These antibodies possess an antigen-binding arm and an arm which binds a cytotoxic agent or a radionuclide chelator, such as EOTUBE, DPTA, DOTA, or TETA. In certain embodiments, the bispecific antibody specifically binds NOTCH1, as well as at least one additional NOTCH receptor selected from the group consisting of NOTCH2, NOTCH3, and NOTCH4 or a NOTCH ligand selected from the group consisting of Jagged1, Jagged2, DLL1, DLL3, and DLL4.

Techniques for making bispecific antibodies are known by those skilled in the art, see for example, Millstein et al., 1983, Nature, 305:537-539; Brennan et al., 1985, Science, 229:81; Suresh et al., 1986, Methods in Enzymol., 121:120; Traunecker et al., 1991, EMBO J., 10:3655-3659; Shalaby et al., 1992, J. Exp. Med., 175:217-225; Kostelny et al., 1992, J. Immunol., 148:1547-1553; Gruber et al., 1994, J. Immunol., 152:5368; and U.S. Pat. No. 5,731,168). Bispecific antibodies can be intact antibodies or antibody fragments. Antibodies with more than two valencies are also contemplated. For example, trispecific antibodies can be prepared (Tutt et al., 1991, J. Immunol., 147:60). Thus, in certain embodiments the antibodies to NOTCH1 are multispecific.

In certain embodiments, the NOTCH1-binding antibody described herein may be monospecific. For example, in certain embodiments, each of the one or more antigen-binding sites that an antibody contains is capable of binding (or binds) a homologous epitope on NOTCH1. In certain embodiments, an antigen-binding site of a monospecific antibody described herein is capable of binding (or binds) NOTCH1 and a second NOTCH such as NOTCH2, NOTCH3 or NOTCH4 (i.e., the same epitope is found on NOTCH1 and, for example, on NOTCH2).

In certain embodiments, the NOTCH1-binding antibody is an antibody fragment. Antibody fragments may have different functions or capabilities than intact antibodies; for example, antibody fragments can have increased tumor penetration. Various techniques are known for the production of antibody fragments including, but not limited to, proteolytic digestion of intact antibodies. In some embodiments, antibody fragments include a F(ab′)2 fragment produced by pepsin digestion of an antibody molecule. In some embodiments, antibody fragments include a Fab fragment generated by reducing the disulfide bridges of an F(ab′)2 fragment. In other embodiments, antibody fragments include a Fab fragment generated by the treatment of the antibody molecule with papain and a reducing agent. In certain embodiments, antibody fragments are produced recombinantly. In some embodiments, antibody fragments include Fv or single chain Fv (scFv) fragments. Fab, Fv, and scFv antibody fragments can be expressed in and secreted from E. coli or other host cells, allowing for the production of large amounts of these fragments. In some embodiments, antibody fragments are isolated from antibody phage libraries as discussed herein. For example, methods can be used for the construction of Fab expression libraries (Huse et al., 1989, Science, 246:1275-1281) to allow rapid and effective identification of monoclonal Fab fragments with the desired specificity for a NOTCH1 protein or derivatives, fragments, analogs or homologs thereof. In some embodiments, antibody fragments are linear antibody fragments as described in U.S. Pat. No. 5,641,870. In certain embodiments, antibody fragments are monospecific or bispecific. In certain embodiments, the NOTCH1-binding antibody is a scFv. Various techniques can be used for the production of single-chain antibodies specific to NOTCH1 (see, e.g., U.S. Pat. No. 4,946,778).

It can further be desirable, especially in the case of antibody fragments, to modify an antibody in order to increase its serum half-life. This can be achieved, for example, by incorporation of a salvage receptor binding epitope into the antibody fragment by mutation of the appropriate region in the antibody fragment or by incorporating the epitope into a peptide tag that is then fused to the antibody fragment at either end or in the middle (e.g., by DNA or peptide synthesis).

For the purposes of the present invention, it should be appreciated that modified antibodies, or fragments thereof, can comprise any type of variable region that provides for the association of the antibody with a membrane proximal region of the extracellular domain of NOTCH1. In this regard, the variable region may be derived from any type of mammal that can be induced to mount a humoral response and generate immunoglobulins against a desired antigen (e.g., NOTCH1). As such, the variable region of the modified antibodies can be, for example, of human, murine, non-human primate (e.g. cynomolgus monkeys, macaques, etc.) or lupine origin. In some embodiments, both the variable and constant regions of the modified immunoglobulins are human. In other embodiments, the variable regions of compatible antibodies (usually derived from a non-human source) can be engineered or specifically tailored to improve the binding properties or reduce the immunogenicity of the molecule. In this respect, variable regions useful in the present invention can be humanized or otherwise altered through the inclusion of imported amino acid sequences.

In some embodiments, the variable domains in both the heavy and light chains are altered by at least partial replacement of one or more CDRs and, if necessary, by partial framework region replacement and sequence modification. Although the CDRs may be derived from an antibody of the same class or even subclass as the antibody from which the framework regions are derived, it is envisaged that the CDRs will be derived from an antibody of different class and preferably from an antibody from a different species. It may not be necessary to replace all of the CDRs with all of the CDRs from the donor variable region to transfer the antigen binding capacity of one variable domain to another. Rather, it may only be necessary to transfer those residues that are necessary to maintain the activity of the antigen binding site.

Alterations to the variable region notwithstanding, those skilled in the art will appreciate that the modified antibodies of this invention may comprise antibodies (e.g., full-length antibodies or antigen-binding fragments thereof) in which at least a fraction of one or more of the constant region domains has been deleted or otherwise altered so as to provide desired biochemical characteristics, such as increased cancer cell localization, increased tumor penetration, reduced serum half-life or increased serum half-life, when compared with an antibody of approximately the same immunogenicity comprising a native or unaltered constant region. In some embodiments, the constant region of the modified antibodies comprises a human constant region. Modifications to the constant region include additions, deletions or substitutions of one or more amino acids in one or more domains. The modified antibodies disclosed herein may comprise alterations or modifications to one or more of the three heavy chain constant domains (CH1, CH2 or CH3) and/or to the light chain constant domain (CL). In some embodiments, one or more domains are partially or entirely deleted from the constant regions of the modified antibodies. In other embodiments, the entire CH2 domain is removed (ΔCH2 constructs). In some embodiments, the omitted constant region domain is replaced by a short amino acid spacer (e.g., 10 aa residues) that provides some of the molecular flexibility typically imparted by the absent constant region.

In some embodiments, the modified antibodies are engineered to fuse the CH3 domain directly to the hinge region of the antibody. In other embodiments, a peptide spacer is inserted between the hinge region and the modified CH2 and/or CH3 domains. For example, constructs may be expressed wherein the CH2 domain has been deleted and the remaining CH3 domain (modified or unmodified) is joined to the hinge region with a 5-20 amino acid spacer. Such a spacer may be added to ensure that the regulatory elements of the constant domain remain free and accessible or that the hinge region remains flexible. However, it should be noted that amino acid spacers may, in some cases, prove to be immunogenic and elicit an unwanted immune response against the construct. Accordingly, in certain embodiments, any spacer added to the construct will be relatively non-immunogenic so as to maintain the desired biological qualities of the modified antibodies.

In some embodiments, the modified antibodies may have only a partial deletion of a constant domain or substitution of a few or even a single amino acid. For example, the mutation of a single amino acid in selected areas of the CH2 domain may be enough to substantially reduce Fc binding and thereby increase cancer cell localization and/or tumor penetration. Similarly, it may be desirable to simply delete that part of one or more constant region domains that control a specific effector function (e.g. complement C1q binding) to be modulated. Such partial deletions of the constant regions may improve selected characteristics of the antibody (serum half-life) while leaving other desirable functions associated with the subject constant region domain intact. Moreover, as alluded to above, the constant regions of the disclosed antibodies may be modified through the mutation or substitution of one or more amino acids that enhances the profile of the resulting construct. In this respect it may be possible to disrupt the activity provided by a conserved binding site (e.g., Fc binding) while substantially maintaining the configuration and immunogenic profile of the modified antibody. In certain embodiments, the modified antibodies comprise the addition of one or more amino acids to the constant region to enhance desirable characteristics such as decreasing or increasing effector function or provide for more cytotoxin or carbohydrate attachment.

In some embodiments, the NOTCH1-binding antibodies provide for altered effector functions that, in turn, affect the biological profile of the administered antibody. For example, in some embodiments, the deletion or inactivation (through point mutations or other means) of a constant region domain may reduce Fc receptor binding of the circulating modified antibody (e.g., NOTCH1 antibody) thereby increasing cancer cell localization and/or tumor penetration. In other embodiments, the constant region modifications increase or reduce the serum half-life of the antibody. In some embodiments, the constant region is modified to eliminate disulfide linkages or oligosaccharide moieties allowing for enhanced cancer cell localization.

In certain embodiments, a NOTCH1-binding antibody does not have one or more effector functions. In some embodiments, the antibody has no ADCC activity, and/or no CDC activity. In certain embodiments, the antibody does not bind to the Fc receptor and/or complement factors. In certain embodiments, the antibody has no effector function.

The present invention further embraces variants and equivalents which are substantially homologous to the chimeric, humanized and/or human antibodies, or antibody fragments thereof, set forth herein. These can contain, for example, conservative substitution mutations, i.e. the substitution of one or more amino acids by similar amino acids.

Thus, the present invention provides methods for generating an antibody that binds NOTCH1. In some embodiments, the method for generating an antibody that binds NOTCH1 comprises using hybridoma techniques. In some embodiments, the method comprises using an extracellular domain of human or mouse NOTCH1 as an immunizing antigen. In some embodiments, the method of generating an antibody that binds NOTCH1 comprises screening a human phage library. The present invention further provides methods of identifying an antibody that binds NOTCH1. In some embodiments, the antibody is identified by screening for binding to NOTCH1 with flow cytometry (FACS). In some embodiments, the antibody is identified by screening for inhibition or blocking of NOTCH1 activation. In some embodiments, the antibody is identified by screening for inhibition or blocking of NOTCH1 signaling.

In certain embodiments, the antibodies described herein are isolated. In certain embodiments, the antibodies described herein are substantially pure.

In certain embodiments, recombinant expression vectors are used to amplify and express DNA encoding NOTCH1-binding antibodies. For example, recombinant expression vectors can be replicable DNA constructs which have synthetic or cDNA-derived DNA fragments encoding a polypeptide chain of a NOTCH1-binding antibody, operatively linked to suitable transcriptional or translational regulatory elements derived from mammalian, microbial, viral or insect genes. A transcriptional unit generally comprises an assembly of (1) a genetic element or elements having a regulatory role in gene expression, for example, transcriptional promoters or enhancers, (2) a structural or coding sequence which is transcribed into mRNA and translated into protein, and (3) appropriate transcription and translation initiation and termination sequences. Regulatory elements can include an operator sequence to control transcription. The ability to replicate in a host, usually conferred by an origin of replication, and a selection gene to facilitate recognition of transformants can additionally be incorporated. DNA regions are “operatively linked” when they are functionally related to each other. For example, DNA for a signal peptide (secretory leader) is operatively linked to DNA for a polypeptide if it is expressed as a precursor which participates in the secretion of the polypeptide; a promoter is operatively linked to a coding sequence if it controls the transcription of the sequence; or a ribosome binding site is operatively linked to a coding sequence if it is positioned so as to permit translation. In some embodiments, structural elements intended for use in yeast expression systems include a leader sequence enabling extracellular secretion of translated protein by a host cell. In other embodiments, where recombinant protein is expressed without a leader or transport sequence, it can include an N-terminal methionine residue. This residue can optionally be subsequently cleaved from the expressed recombinant protein to provide a final product.

The choice of expression control sequence and expression vector depends upon the choice of host. A wide variety of expression host/vector combinations can be employed. Useful expression vectors for eukaryotic hosts include, for example, vectors comprising expression control sequences from SV40, bovine papilloma virus, adenovirus and cytomegalovirus. Useful expression vectors for bacterial hosts include known bacterial plasmids, such as plasmids from E. coli, including pCR1, pBR322, pMB9 and their derivatives, and wider host range plasmids, such as M13 and other filamentous single-stranded DNA phages.

Suitable host cells for expression of a NOTCH1-binding antibody (or a NOTCH1 polypeptide to use as an antigen in the generation of antibodies) include prokaryotes, yeast, insect or higher eukaryotic cells under the control of appropriate promoters. Prokaryotes include gram-negative or gram-positive organisms, for example, E. coli or Bacillus. Higher eukaryotic cells include established cell lines of mammalian origin as described below. Cell-free translation systems could also be employed.

Various mammalian or insect cell culture systems are used to express recombinant polypeptides. Expression of recombinant proteins in mammalian cells can be preferred because such proteins are generally correctly folded, appropriately modified and completely functional. Examples of suitable mammalian host cell lines include COS-7 (monkey kidney-derived), L-929 (murine fibroblast-derived), C127 (murine mammary tumor-derived), 3T3 (murine fibroblast-derived), CHO (Chinese hamster ovary-derived), HeLa (human cervical cancer-derived), BHK (hamster kidney fibroblast-derived) cell lines and variants thereof. Mammalian expression vectors can comprise non-transcribed elements such as an origin of replication, a suitable promoter and enhancer linked to the gene to be expressed, and other 5′ or 3′ flanking non-transcribed sequences, and 5′ or 3′ non-translated sequences, such as necessary ribosome binding sites, a polyadenylation site, splice donor and acceptor sites, and transcriptional termination sequences. Baculovirus systems for production of heterologous proteins in insect cells are well-known to those of skill in the art (see, e.g., Luckow and Summers, 1988, Bio/Technology, 6:47).

The proteins produced by a transformed host can be purified according to any suitable method. Such standard methods include chromatography (e.g., ion exchange, affinity and sizing column chromatography), centrifugation, differential solubility, or by any other standard technique for protein purification. Affinity tags such as hexahistidine, maltose binding domain, influenza coat sequence and glutathione-S-transferase can be attached to the protein to allow easy purification by passage over an appropriate affinity column. Isolated proteins can also be physically characterized using such techniques as proteolysis, high performance liquid chromatography (HPLC), nuclear magnetic resonance and x-ray crystallography.

In some embodiments, supernatants from expression systems which secrete recombinant protein into culture media can be first concentrated using a commercially available protein concentration filter, for example, an Amicon or Millipore Pellicon ultrafiltration unit. Following the concentration step, the concentrate can be applied to a suitable purification matrix. In other embodiments, an anion exchange resin can be employed, for example, a matrix or substrate having pendant diethylaminoethyl (DEAE) groups. The matrices can be acrylamide, agarose, dextran, cellulose or other types commonly employed in protein purification. In some embodiments, a cation exchange step can be employed. Suitable cation exchangers include various insoluble matrices comprising sulfopropyl or carboxymethyl groups. In some embodiments, a hydroxyapatite (CHT) media can be employed, including but not limited to, ceramic hydroxyapatite. In some embodiments, one or more reversed-phase HPLC steps employing hydrophobic RP-HPLC media, e.g., silica gel having pendant methyl or other aliphatic groups, can be employed to further purify a recombinant protein. Some or all of the foregoing purification steps, in various combinations, can also be employed to provide a homogeneous recombinant protein.

In some embodiments, recombinant protein produced in bacterial culture can be isolated, for example, by initial extraction from cell pellets, followed by one or more concentration, salting-out, aqueous ion exchange or size exclusion chromatography steps. HPLC can be employed for final purification steps. Microbial cells employed in expression of a recombinant protein can be disrupted by any convenient method, including freeze-thaw cycling, sonication, mechanical disruption, or use of cell lysing agents.

Methods known in the art for purifying antibodies also include, for example, those described in U.S. Patent Pub. Nos. 2008/0312425; 2008/0177048; and 2009/0187005.

In certain embodiments, the NOTCH1-binding antibodies can be used in any one of a number of conjugated (i.e. an immunoconjugate or radioconjugate) or non-conjugated forms. In certain embodiments, the antibodies can be used in a non-conjugated form to harness the subject's natural defense mechanisms including complement-dependent cytotoxicity and antibody dependent cellular toxicity to eliminate the malignant or cancer cells.

In some embodiments, the NOTCH1-binding antibody is conjugated to a cytotoxic agent. In some embodiments, the cytotoxic agent is a chemotherapeutic agent including, but not limited to, methotrexate, adriamycin, doxorubicin, melphalan, mitomycin C, chlorambucil, daunorubicin or other intercalating agents. In some embodiments, the cytotoxic agent is an enzymatically active toxin of bacterial, fungal, plant or animal origin, or fragments thereof, including, but not limited to, diphtheria A chain, nonbinding active fragments of diphtheria toxin, exotoxin A chain, ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Saponaria officinalis inhibitor, gelonin, mitogellin, restrictocin, phenomycin, enomycin, and the tricothecenes. In some embodiments, the cytotoxic agent is a radioisotope to produce a radioconjugate or a radioconjugated antibody. A variety of radionuclides are available for the production of radioconjugated antibodies including, but not limited to, ⁹⁰Y, ¹²⁵I, ¹³¹I, ¹²³I, ¹¹¹In, ¹³¹In, ¹⁰⁵Rh, ¹⁵³Sm, ⁶⁷Cu, ⁶⁷Ga, ¹⁶⁶Ho, ¹⁷⁷Lu, ¹⁸⁶Re, ¹⁸⁸Re and ²¹²Bi. Conjugates of an antibody and one or more small molecule toxins, such as a calicheamicin, maytansinoids, a trichothene, and CC1065, and the derivatives of these toxins that have toxin activity, can also be used. Conjugates of an antibody and cytotoxic agent are made using a variety of bifunctional protein-coupling agents such as N-succinimidyl-3-(2-pyridyidithiol) propionate (SPDP), iminothiolane (IT), bifunctional derivatives of imidoesters (such as dimethyl adipimidate HICL), active esters (such as disuccinimidyl suberate), aldehydes (such as glutaraldehyde), bis-azido compounds (such as bis(p-azidobenzoyi) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene).

Heteroconjugate antibodies are also within the scope of the present invention. Heteroconjugate antibodies are composed of two covalently joined antibodies. Such antibodies have, for example, been proposed to target immune cells to unwanted cells (U.S. Pat. No. 4,676,980). It is contemplated that the antibodies can be prepared in vitro using known methods in synthetic protein chemistry, including those involving cross-linking agents.

### III. NOTCH MUTATIONS AND/OR ACTIVATED NOTCH AND METHODS OF IDENTIFICATION

It has been reported that that approximately 8% of CLLs have NOTCH1 activating mutations, which increases up to 30% as CLL progresses toward Richter's syndrome (also known as diffuse large B-cell lymphoma—DLBCL). In addition, NOTCH1 activation at CLL diagnosis was observed to be an independent predictor of poor survival (Fabbri et al., 2011, JEM, 208:1389-1401. For mantle cell lymphoma it has been reported that about 12% of clinical samples contain a NOTCH1 coding sequence mutation that cluster in the PEST domain and predominantly consist of truncating or frame-shifting mutations. These NOTCH1 mutations were also associated with poor overall survival (Kridel et al., 2012, Blood, 119:1963-1971). Thus, it is beneficial to identify hematologic cancers that contain an activating NOTCH1 mutation and/or detect activated NOTCH1 in hematologic cancer.

In some embodiments of the present invention, the hematologic cancer comprises cancer cells in which NOTCH1 is activated. In certain embodiments, the hematologic cancer comprises a NOTCH1 mutation (i.e., a mutation in the NOTCH1 gene). In certain embodiments, the mutation is in a portion of the NOTCH1 gene encoding the NOTCH1 receptor (i.e., in the NOTCH1 coding sequence). In some embodiments, the mutation is an activating NOTCH1 mutation. By way of non-limiting example, the mutation may be in the sequence of the NOTCH1 gene that encodes the PEST domain and/or the HD domain of NOTCH1. In some embodiments, the mutation in the human NOTCH1 gene results in a truncation of the encoded NOTCH1 protein in the PEST domain. One non-limiting example of such a mutation is a deletion at position 7444 of NOTCH1 (e.g., in DLBCL cells). In some alternative embodiments, the mutation in the human NOTCH1 gene results in a truncation of the encoded NOTCH1 protein in the HD domain. For example, but not limited to a truncation at amino acid position 2482 of NOTCH1. In some further embodiments, the mutation is in the sequence of the NOTCH1 gene that encodes an EGF repeat of the NOTCH1 extracellular domain. In some embodiments, the mutation in the human NOTCH1 gene results in an amino acid substitution in an EGF repeat of the NOTCH1 extracellular domain. In one embodiment, the mutation is in a non-ligand binding EGF repeat. In one embodiment, the mutation is in EGF repeat 36. In one embodiment, the mutation is a substitution at residue 4168 of the NOTCH1 gene, for example but not limited to C4168A. In one embodiment, the mutation is a missense mutation resulting in an amino acid substitution. In one embodiment, the mutation results in an amino acid substitution at position 1390 of NOTCH1. In one embodiment, the amino acid substitution is selected from the group consisting of P1390T, P1390A and P1390S.

In a hematologic cancer, some, all, a majority, a minority, or none of the cancer cells may comprise a mutation in NOTCH1. If a cancer is said to “comprise” a mutation, at least some of the cancer cells have such mutation. In some embodiments, the mutation is heterozygous. In some embodiments, the mutation is somatic. In certain embodiments, about 1% or more of the cancer cells are identified as having the mutation, about 5% or more of the cancer cells are identified as having the mutation, about 10% or more of the cancer cells are identified as having the mutation, about 20% or more of the cancer cells are identified as having the mutation, or about 50% or more of the cancer cells are identified as having the mutation.

Methods for detecting a NOTCH1 mutation (or determining whether NOTCH1 is activated) for purposes of determining whether to select a patient for treatment with a NOTCH1-binding antibody may, in certain embodiments, comprise a step of obtaining a body sample from the subject. In certain embodiments, the sample is whole blood, serum, plasma, or tissue.

Methods for detecting a NOTCH1 mutation (or determining whether NOTCH1 is activated) in a hematologic cancer may comprise any method that determines the presence of the mutation at either the nucleic acid or protein level. Such methods are well known in the art and include but are not limited to Western blots, Northern blots, Southern blots, ELISA, immunoprecipitation, immunofluorescence, flow cytometry, immunocytochemistry, nucleic acid sequencing, nucleic acid hybridization techniques, nucleic acid reverse transcription methods, and nucleic acid amplification methods, such as PCR. In certain embodiments, the analyses can be based on PCR-based assays, using for instance one or more of the following approaches: size fractionation by gel electrophoresis, direct sequencing, single-strand conformation polymorphism (SSCP), high pressure liquid chromatography (including partially denaturing HPLC), allele-specific hybridization, amplification refractory mutation screening, NOTCH mutation screening by oligonucleotide microarray, restriction fragment polymorphism, MALDI-TOF mass spectrometry, or various related technologies. In some embodiments, the methods comprise Sanger sequencing or next generation sequencing.

In some embodiments, mutations in NOTCH1 (which may result in increased NOTCH1 signaling) are detected on a protein level using, for example, antibodies that are directed against mutated NOTCH1 receptors or downstream NOTCH1 targets. These antibodies can be used in various methods such as Western blot, ELISA, immunoprecipitation, immunohistochemistry, or immunocytochemistry techniques.

Methods for detecting the level of NOTCH ICD in tumor cells can comprise any method that detects the presence of a NOTCH ICD polypeptide in a biological sample. Such methods are well known in the art and include, but are not limited to, western blots, slot blots, ELISA, immunoprecipitation, immunofluorescence, flow cytometry, immunocytochemistry, immunohistochemistry (IHC), and mass spectroscopy. In one embodiment, the level of NOTCH ICD in a tumor sample is determined using IHC.

In some embodiments, the level of NOTCH1-ICD is determined using an agent that specifically binds to NOTCH1-ICD. Any molecular entity that displays specific binding to NOTCH1-ICD can be employed to determine the level of NOTCH1-ICD in a sample. Specific binding agents include, but are not limited to, antibodies, antibody mimetics, and polynucleotides (e.g., aptamers). One of skill understands that the degree of specificity required is determined by the particular assay used to detect NOTCH1-ICD. For example, an agent that specifically binds to both full length NOTCH and NOTCH1-ICD can be used in a method that involves the separation of polypeptides based on their size, e.g. Western blot. In some embodiments, a method employs an agent that specifically binds to NOTCH1-ICD to determine the level of NOTCH1-ICD in a sample. In some embodiments, the agent is an antibody. Anti-NOTCH ICD-specific antibodies can be generated according to any method known to one of skill in the art. In some embodiments, an anti-NOTCH1-ICD specific antibody specifically binds to NOTCH1-ICD, but does not significantly bind to NOTCH1. The anti-NOTCH1-ICD antibody can be a monoclonal antibody, polyclonal antibody, humanized antibody, human antibody, chimeric antibody, or an antigen-binding fragment thereof. In some embodiments, the antibody specifically binds to NOTCH1-ICD in a fixed and embedded tissue sample. The tissue sample can be a formalin fixed tissue sample. The tissue sample can be a paraffin embedded tissue sample. In some embodiments, the antibody specifically binds to NOTCH1-ICD in a cytospin preparation.

Techniques for detecting antibody binding are well known in the art. Antibody binding to a NOTCH1-ICD can be detected through the use of chemical reagents that generate a detectable signal that corresponds to the level of antibody binding and, accordingly, to the amount of NOTCH1-ICD. In some embodiments, NOTCH1-ICD antibody binding is detected through the use of a secondary antibody that is conjugated to a labeled polymer. Examples of labeled polymers include, but are not limited to, polymer-enzyme conjugates. The enzymes in these complexes are typically used to catalyze the deposition of a chromogen at the antigen-antibody binding site, thereby resulting in cell staining that corresponds to expression level of the mutation of interest. Enzymes of particular interest include horseradish peroxidase (HRP) and alkaline phosphatase (AP). Commercial antibody detection systems, such as, for example the Dako Envision+ system (Dako North America, Inc., Carpinteria, Calif.) and Mach 3 system (Biocare Medical, Walnut Creek, Calif.) are available to one of skill in the art.

Detection of antibody binding can be facilitated by coupling the NOTCH1-ICD antibody directly to a detectable label. Examples of detectable labels include various enzymes, prosthetic groups, fluorescent materials, luminescent materials, bioluminescent materials, and radioactive materials. Examples of suitable enzymes include HRP, AP, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; an example of bioluminescent materials include luciferase; and examples of suitable radioactive material include ¹²⁵I, ¹³¹I, ³⁵S, or ³H.

The level of antibody binding to NOTCH1-ICD can be quantified by various methods known in the art, for example, enzyme linked immunosorbent assays (ELISA), immunofluorescence, immunohistochemistry, and radioimmunoassay (RIA). The detection of NOTCH1-ICD levels is not limited to such techniques, and Western blot analyses, dot blot analyses, FACS analyses, and the like may also be used.

In some embodiments, the method comprises determining the amount of NOTCH1-ICD in a subcellular compartment, for example, in the nucleus. In some embodiments, the amount of NOTCH1-ICD in the nucleus is determined by isolating the nuclear protein fraction from a cellular sample. In some embodiments, the amount of NOTCH1-ICD in the nucleus is determined by microscopy. Such nuclear NOTCH1-ICD determination methods can be performed manually or in an automated fashion.

In some embodiments, the level of NOTCH1-ICD in tumor cell nuclei is determined by microscopy. NOTCH1-ICD levels can be determined, for example, by immunofluorescence, immunohistochemistry (IHC), and radioimmunoassay (RIA). In one embodiment, the level of NOTCH1-ICD in the nucleus is determined by IHC. The level of nuclear NOTCH1-ICD in a sample can be expressed by any scoring system known to the skilled artisan.

The amount of NOTCH1-ICD in a tumor sample may be scored based on the intensity of the NOTCH1-ICD specific staining or based on the percentage of NOTCH1-ICD positive cells. In some embodiments, the amount of nuclear NOTCH1-ICD in a sample is expressed as a proportion, e.g., percentage, of cells within the sample that comprise detectable amounts of NOTCH1-ICD. For example, the amount of nuclear NOTCH1-ICD in a sample can be expressed as 10%, 20%, 30%, 40%, etc. of the nuclei in the sample are NOTCH1-ICD positive. In some embodiments, the amount of nuclear NOTCH1-ICD in a sample is determined by assessing the staining intensity of the nuclei in the sample. For example, a sample can be characterized as negative, weakly stained, intermediately stained, or strongly stained based on the NOTCH1-ICD specific staining intensity of the nuclei.

In some embodiments, the amount of NOTCH1-ICD in the nuclei of a sample is determined by assessing both the intensity and frequency of the NOTCH1-ICD specific. In some embodiments, an “H-score” is used to characterize the amount of NOTCH1-ICD in a sample. A semi-quantitative intensity scale ranging from 0 for no staining to 3+ for the most intense staining is used to assign a staining intensity score to nuclei in the sample. The number of nuclei falling into each category, i.e., 0, 1+, 2+, and 3+ is counted. An H-Score is calculated for staining of the nuclei using the following formula: [(% at 0)*0]+[(% at 1+)*1]+[(% at 2+)*2]+[(% at 3+)*3]. The H-score will range from a score of 0 to 300.

Another aspect of the methods described herein is comparing the amount of NOTCH1-ICD detected in a test sample to a predetermined standard, for example, the NOTCH1-ICD level of a control sample. A control sample can be a sample obtained from the patient in a manner similar to the test samples wherein the control sample does not comprise hematologic cancer cells. A control sample can also be obtained in a manner similar to the test samples from a subject that does not have a cancer.

In some embodiments, the method comprises comparing the level of NOTCH1-ICD to a predetermined standard, or reference level, or control level. The terms “predetermined standard”, “reference level”, and “control level” are used interchangeably herein. In some embodiments, a predetermined standard is a baseline amount of NOTCH1-ICD measured in a comparable control sample, e.g., a sample that does not comprise cancer cells. In some embodiments, a predetermined standard is a baseline amount of NOTCH1-ICD measured in a sample comprising cancer cells that do not express elevated levels of NOTCH1-ICD. In some embodiments, a predetermined standard is a baseline amount of NOTCH1-ICD measured in a sample comprising cancer cells that do not respond to treatment with a NOTCH1 antagonist or inhibitor, e.g., an anti-NOTCH1 antibody. In some embodiments, a predetermined standard is a baseline amount of NOTCH1-ICD measured in an isolated cell line. The cell line can be derived from a cancer sample. The cell line can be recombinantly manipulated to express NOTCH1 or an increased amount of NOTCH1-ICD.

The present invention further provides isolated polynucleotides comprising a sequence that encodes a mutant human NOTCH1 receptor, wherein the sequence comprises a deletion at position 7444 of the human NOTCH1 gene. The present invention further provides isolated polynucleotides comprising a sequence that encodes a mutant human NOTCH1 receptor, wherein the sequence comprises a substitution at position 4168 of the human NOTCH1 gene. Isolated polypeptides encoded by such polynucleotides are also provided. Recombinant vectors comprising the polynucleotides, optionally operably linked to a promoter sequence, and isolated or recombinant cells comprising the polynucleotides are also provided.

### IV. METHODS OF USE AND PHARMACEUTICAL COMPOSITIONS

The NOTCH1-binding antibodies of the invention are useful in a variety of applications including, but not limited to, therapeutic treatment methods, such as the treatment of hematologic cancers. In certain embodiments, the antibodies are useful for modulating NOTCH1 activity, inhibiting NOTCH1 activity, inhibiting or blocking NOTCH1/NOTCH ligand interactions, inhibiting NOTCH1 signaling, and/or inhibiting NOTCH1 activation. In some embodiments, the antibodies are useful for blocking cleavage of NOTCH1, for inhibiting cleavage with the membrane proximal region, for inhibiting cleavage at the S2 site within the membrane proximal region, for inhibiting release of the intracellular domain of NOTCH1. In some embodiments, the antibodies are useful in inhibiting cancer cell growth, reducing cancer cell volume, reducing the cancer cell population, reducing the tumorigenicity of a hematologic cancer, reducing the frequency of cancer stem cells in a hematologic cancer, reducing the frequency of leukemia-initiating cells in a hematologic cancer, inducing death of cancer cells, and/or inducing differentiation. The methods of use may be in vitro, ex vivo, or in vivo methods. In certain embodiments, the NOTCH1-binding antibody is an antagonist of NOTCH1. In certain embodiments, the antibody is an antagonist of a NOTCH signaling pathway. In some embodiments, the antibody is an antagonist of NOTCH1 activation.

In certain embodiments, the NOTCH1-binding antibodies described herein are used in the treatment of a disease associated with NOTCH signaling and activation. In particular embodiments, the disease is a disease associated with a NOTCH signaling pathway. In particular embodiments, the disease is a disease associated with a constitutively activated NOTCH1. In some embodiments, cancer cell growth is associated with a NOTCH signaling pathway. In some embodiments, cancer cell growth is associated with NOTCH1 activation. In some embodiments, the disease is a hematologic cancer. In certain embodiments, the hematologic cancer is chronic lymphocytic leukemia, hairy cell leukemia, chronic myelogenous leukemia, non-Hodgkin lymphoma, diffuse large B-cell lymphoma, mantle cell lymphoma, or cutaneous T-cell lymphoma. In some embodiments, the disease is chronic lymphocytic leukemia. In certain embodiments, the hematologic cancer is Richter's transformation or Richter's syndrome. NOTCH1-binding antibodies may also be used for preventing or inhibiting Richter's transformation, as well as treating the condition. Other non-limiting examples of hematologic cancers which may be treated using the NOTCH1-binding antibodies and methods provided herein include NK-cell leukemia, splenic marginal zone lymphoma, and follicular lymphoma.

In certain embodiments, the subject has a hematologic cancer that is refractory (e.g., chemorefractory). For example, the subject may have been treated with one or more courses of other anti-cancer therapeutic agents or therapies (e.g., chemotherapy) prior to administration of the NOTCH1-binding antibody.

The present invention further provides methods for inhibiting growth of a hematologic cancer using the NOTCH1-binding antibodies described herein. In certain embodiments, the method of inhibiting growth of a hematologic cancer comprises contacting tumor cells with a NOTCH1-binding antibody in vitro. For example, an immortalized cell line or a cancer cell line that expresses NOTCH1 on the cell surface is cultured in medium to which is added the antibody to inhibit cancer cell growth.

In some embodiments, cancer cells are isolated from a patient sample such as, for example, a tissue biopsy, pleural effusion, or blood sample and cultured in medium to which is added a NOTCH1-binding antibody to inhibit cancer cell growth.

In some embodiments, the method of inhibiting growth of a hematologic cancer comprises contacting the cancer cells with a NOTCH1-binding antibody in vivo. In certain embodiments, contacting cancer cells with a NOTCH1-binding antibody is undertaken in an animal model.

In some embodiments, NOTCH1-binding antibodies are administered soon after the injection of the hematologic cancer cells to study the effect of the NOTCH1-binding antibodies upon engraftment of the cancer cells. In some embodiments, NOTCH1-binding antibodies are administered prior to the injection of the hematologic cancer cells. In some embodiments, NOTCH1-binding antibodies are administered after the hematologic cancer cells have engrafted into the mice to study the effect of the NOTCH1-binding antibodies upon an established hematologic cancer. In some embodiments, NOTCH1-binding antibodies are administered after the hematologic cancer cells have engrafted into preconditioned mice to study the effect of the NOTCH1-binding antibodies upon an established hematologic cancer. In some embodiments, the NOTCH1-binding antibodies are administered to the mice 1 day, 2 days, 3 days, 4 days, 5 days, etc. before the hematologic cancer cells are injected into the mice. In some embodiments, the NOTCH1-binding antibodies are administered to the mice 1 week, 2 weeks, 3 weeks, 4 weeks, 5 weeks, 6 weeks, etc. after engraftment of the hematologic cancer cells. After administration of NOTCH1-binding antibodies, the mice are observed for inhibition of cancer cell engraftment and/or inhibition of cancer cell growth. In some embodiments, cancer stem cells are isolated from a patient sample such as, for example, a tissue biopsy, pleural effusion, or blood sample and injected into immunocompromised mice that are then administered a NOTCH1-binding antibody to inhibit cancer cell growth. In some embodiments, cancer stem cells are isolated from a patient sample such as, for example, a tissue biopsy, pleural effusion, or blood sample and injected into irradiated, preconditioned immunocompromised mice that are then administered a NOTCH1-binding antibody to inhibit cancer cell growth. In some embodiments, the NOTCH1-binding antibody is administered at the same time or shortly after introduction of cancer cells into the animal to prevent cancer cell growth. In some embodiments, the NOTCH1-binding antibody is administered as a therapeutic after the cancer cells have engrafted and established a hematologic cancer. In some embodiments, the hematologic cancer cells comprise cancer stem cells. In some embodiments, the hematologic cancer cells comprise leukemia-initiating cells.

The invention provides methods of inhibiting the growth of a hematologic cancer in a subject, the method comprising administering to the subject a therapeutically effective amount of an antibody that specifically binds a non-ligand binding membrane proximal region of the extracellular domain of human NOTCH1. In some embodiments, the hematologic cancer comprises cancer stem cells. In some embodiments, the hematologic cancer comprises leukemia-initiating cells. In some embodiments, the method comprises targeting the cancer stem cells with the NOTCH1 antibodies described herein. In certain embodiments, the method of inhibiting growth of a hematologic cancer comprises administering a therapeutically effective amount of the NOTCH1 antibodies described herein.

In certain embodiments, the method of inhibiting growth of a hematologic cancer comprises reducing the frequency of cancer stem cells in the cancer, reducing the number of cancer stem cells in the cancer, reducing the tumorigenicity of the cancer, and/or reducing the tumorigenicity of the cancer by reducing the number or frequency of cancer stem cells in the cancer. In some embodiments, the method of inhibiting growth of a hematologic cancer comprises inhibiting the activity of a NOTCH1 receptor. In certain embodiments, the hematologic cancer includes, but is not limited to, chronic lymphocytic leukemia, hairy cell leukemia, chronic myelogenous leukemia, non-Hodgkin lymphoma, diffuse large B-cell lymphoma, mantle cell lymphoma, or cutaneous T-cell lymphoma. In some embodiments, the disease is chronic lymphocytic leukemia.

In certain embodiments, the method of treating a hematologic cancer comprises administering a therapeutically effective amount of an antibody conjugated to a cytotoxic moiety that specifically binds a non-ligand binding membrane proximal region of the extracellular domain of a human NOTCH1 receptor and inhibits cancer growth. In certain embodiments, the method of treating a hematologic cancer comprises administering a therapeutically effective amount of an antibody of any of the aspects and/or embodiments, as well as other aspects and/or embodiments described herein, in combination with radiation therapy. In certain embodiments, the method of treating a hematologic cancer comprises administering a therapeutically effective amount of an antibody of any of the aspects and/or embodiments, as well as other aspects and/or embodiments described herein, in combination with chemotherapy. In certain embodiments, the method of treating a hematologic cancer comprises administering a therapeutically effective amount of an antibody that specifically binds a non-ligand binding membrane proximal region of the extracellular domain of a human NOTCH1 receptor and inhibits cancer growth wherein the hematologic cancer includes, but is not limited to, chronic lymphocytic leukemia, hairy cell leukemia, chronic myelogenous leukemia, non-Hodgkin lymphoma, diffuse large B-cell lymphoma, mantle cell lymphoma, or cutaneous T-cell lymphoma.

In another aspect, the present invention provides a method of treating a hematologic cancer in a subject in need thereof comprising administering to a subject a therapeutically effective amount of an antibody that specifically binds a non-ligand binding membrane proximal region of the extracellular domain of a human NOTCH1 receptor protein and inhibits cancer growth in the subject. In certain embodiments, the method of treating a hematologic cancer comprises administering a therapeutically effective amount of a monoclonal antibody. In certain embodiments, the method of treating a hematologic cancer comprises administering a therapeutically effective amount of a chimeric antibody. In certain embodiments, the method of treating a hematologic cancer comprises administering a therapeutically effective amount of a humanized antibody. In certain embodiments, the method of treating a hematologic cancer comprises administering a therapeutically effective amount of a human antibody.

In certain embodiments, the method of inhibiting growth of a hematologic cancer comprises administering to a subject a therapeutically effective amount of a NOTCH1-binding antibody. In certain embodiments, the subject is a human. In certain embodiments, the subject has a hematologic cancer. In certain embodiments, the subject has had cancer cells removed. In some embodiments, the NOTCH1-binding antibody is antibody 52M51. In some embodiments, the NOTCH1-binding antibody is a humanized version of 52M51.

In certain embodiments, the hematologic cancer cell expresses NOTCH1 to which the NOTCH1-binding antibody binds. In certain embodiments, the tumor over-expresses a human NOTCH1. In some embodiments, the NOTCH1-binding antibody binds NOTCH1 and inhibits or reduces growth of the hematologic cancer. In some embodiments, the NOTCH1-binding antibody binds NOTCH1, interferes with NOTCH1/NOTCH ligand interactions, and inhibits or reduces growth of the hematologic cancer. In some embodiments, the NOTCH1-binding antibody binds NOTCH1, inhibits NOTCH activation and inhibits or reduces growth of the hematologic cancer. In some embodiments, the NOTCH1-binding antibody binds NOTCH1, and reduces the frequency of cancer stem cells in the hematologic cancer. In some embodiments, the NOTCH1-binding antibody binds a constitutively activated NOTCH1 and inhibits NOTCH1 activity.

In certain embodiments, the hematologic cancer is chronic lymphocytic leukemia, hairy cell leukemia, chronic myelogenous leukemia, non-Hodgkin lymphoma, diffuse large B-cell lymphoma, mantle cell lymphoma, or cutaneous T-cell lymphoma. In some embodiments, the disease is chronic lymphocytic leukemia. In certain embodiments, the hematologic cancer is hairy cell leukemia. In certain embodiments, the hematologic cancer is chronic myelogenous leukemia. In certain embodiments, the hematologic cancer is non-Hodgkin lymphoma. In certain embodiments, the hematologic cancer is mantle cell lymphoma. In certain embodiments, the hematologic cancer is cutaneous T-cell lymphoma. In certain embodiments, the subject is a human.

The present invention further provides methods for treating a hematologic cancer using the NOTCH1-binding antibodies described herein. In certain embodiments, the hematologic cancer is characterized by cells expressing NOTCH1 to which the NOTCH1-binding antibody binds. In certain embodiments, the hematologic cancer over-expresses human NOTCH1. In certain embodiments, the hematologic cancer is characterized by cells expressing NOTCH1, wherein the NOTCH1 antibody interferes with NOTCH ligand-induced NOTCH signaling and/or activation. In some embodiments, the NOTCH1-binding antibody binds NOTCH1 and inhibits or reduces growth of the hematologic cancer. In some embodiments, the NOTCH1-binding antibody binds NOTCH1, interferes with NOTCH1/NOTCH ligand interactions and inhibits or reduces growth of the hematologic cancer. In some embodiments, the NOTCH1-binding antibody binds NOTCH1, inhibits NOTCH1 activation and inhibits or reduces growth of the hematologic cancer. In some embodiments, the NOTCH-binding antibody binds NOTCH, and reduces the frequency of cancer stem cells in the hematologic cancer.

The present invention provides for methods of treating a hematologic cancer comprising administering a therapeutically effective amount of a NOTCH1-binding antibody to a subject (e.g., a subject in need of treatment). In certain embodiments, the subject is a human. In certain embodiments, the subject has a hematologic cancer. In certain embodiments, the subject has had cancer cells removed. In some embodiments, the NOTCH1-binding antibody is antibody 52M51. In some embodiments, the NOTCH1-binding antibody is a humanized version of 52M51.

In certain embodiments, the hematologic cancer is a cancer selected from the group consisting of chronic lymphocytic leukemia, hairy cell leukemia, chronic myelogenous leukemia, non-Hodgkin lymphoma, diffuse large B-cell lymphoma, mantle cell lymphoma, and cutaneous T-cell lymphoma. In some embodiments, the hemotologic cancer is chronic lymphocytic leukemia. In certain embodiments, the hematologic cancer is hairy cell leukemia. In certain embodiments, the hematologic cancer is chronic myelogenous leukemia. In certain embodiments, the hematologic cancer is non-Hodgkin lymphoma. In certain embodiments, the hematologic cancer is mantle cell lymphoma. In certain embodiments, the hematologic cancer is cutaneous T-cell lymphoma. In certain embodiments, the subject is a human.

The invention also provides a method of inhibiting NOTCH signaling or NOTCH activation in a cell comprising contacting the cell with an effective amount of a NOTCH1-binding antibody. In certain embodiments, the cell is a hematologic cancer cell. In certain embodiments, the method is an in vivo method wherein the step of contacting the cancer cell with the NOTCH-binding antibody comprises administering a therapeutically effective amount of the NOTCH1-binding antibody to the subject. In some embodiments, the method is an in vitro or ex vivo method. In certain embodiments, the NOTCH1-binding antibody inhibits NOTCH signaling. In some embodiments, the NOTCH1-binding antibody inhibits NOTCH activation. In certain embodiments, the NOTCH1-binding antibody interferes with a NOTCH1/NOTCH ligand interaction. In certain embodiments, the NOTCH1-binding antibody inhibits NOTCH activation of at least one additional NOTCH receptor selected from the group consisting of NOTCH2, NOTCH3, and NOTCH4. In some embodiments, the NOTCH1-binding antibody is an antibody. In some embodiments, the NOTCH1-binding antibody is antibody 52M51. In some embodiments, the NOTCH1-binding antibody is a humanized version of 52M51.

In addition, the invention provides a method of reducing the tumorigenicity of a hematologic cancer in a subject, comprising administering a therapeutically effective amount of a NOTCH1-binding antibody to the subject. In certain embodiments, the hematologic cancer comprises cancer stem cells. In certain embodiments, the hematologic cancer comprises leukemia-initiating cells. In certain embodiments, the frequency of cancer stem cells in the hematologic cancer is reduced by administration of the NOTCH1-binding antibody. The invention also provides a method of reducing the frequency of cancer stem cells in a hematologic cancer, comprising contacting the cancer cells with an effective amount of a NOTCH1-binding antibody. In some embodiments, the NOTCH1-binding antibody is antibody 52M51. In some embodiments, the NOTCH1-binding antibody is a humanized version of 52M51.

The invention also provides a method of treating a disease or disorder in a subject, wherein the disease or disorder is characterized by an increased level of cancer stem cells and/or progenitor cells. In some embodiments, the treatment methods comprise administering a therapeutically effective amount of the NOTCH1-binding antibody to the subject.

The present invention also provides methods of treating a hematologic cancer in a human subject, comprising: (a) determining that the subject's hematologic cancer comprises a NOTCH1 mutation, and (b) administering to the subject (e.g., a subject in need of treatment) a therapeutically effective amount of a Notch 1-binding antibody described herein. In certain embodiments, the subject has had a cancer treated. In certain embodiments, the subject has had a cancer removed. In some embodiments, the NOTCH1-binding antibody is antibody 52M51. In some embodiments, the NOTCH1-binding antibody is a humanized version of 52M51.

The present invention further provides methods of treating a hematologic cancer in a human subject, comprising: (a) selecting a subject for treatment based, at least in part, on the subject having a hematologic cancer that comprises a NOTCH1 mutation, and (b) administering to the subject a therapeutically effective amount of a NOTCH1-binding antibody described herein. In certain embodiments, the subject has had a cancer treated. In certain embodiments, the subject has had a cancer removed. In some embodiments, the NOTCH1-binding antibody is a humanized or chimeric version of the murine antibody 52M51. In some embodiments, the NOTCH1-binding antibody is antibody 52M51-H4L3.

The present invention further provides methods of treating a hematologic cancer in a human subject, comprising: (a) identifying a subject that has a hematologic cancer comprising a NOTCH1 mutation, and (b) administering to the subject a therapeutically effective amount of a NOTCH1-binding antibody described herein. In certain embodiments, the subject has had a cancer treated. In certain embodiments, the subject has had a cancer removed. In some embodiments, the NOTCH1-binding antibody is a humanized or chimeric version of the murine antibody 52M51. In some embodiments, the NOTCH1-binding antibody is antibody 52M51-H4L3.

The present invention further provides pharmaceutical compositions comprising NOTCH1-binding antibodies described herein. These pharmaceutical compositions find use in inhibiting growth of cancer cells, inhibiting growth of a hematologic cancer, and treating a hematologic cancer in human patients.

Formulations are prepared for storage and use by combining a purified antagonist (e.g., antibody) of the present invention with a pharmaceutically acceptable vehicle (e.g., carrier, excipient, etc.) (Remington: The Science and Practice of Pharmacy, 21^{st} Edition, University of the Sciences Philadelphia 2005). Suitable pharmaceutically acceptable vehicles include, but are not limited to, nontoxic buffers such as phosphate, citrate, and other organic acids; salts such as sodium chloride; antioxidants including ascorbic acid and methionine; preservatives such as octadecyldimethylbenzyl ammonium chloride; hexamethonium chloride; benzalkonium chloride; benzethonium chloride; phenol, butyl or benzyl alcohol; alkyl parabens, such as methyl or propyl paraben; catechol; resorcinol; cyclohexanol; 3-pentanol; and m-cresol; low molecular weight polypeptides (less than about 10 amino acid residues); proteins such as serum albumin, gelatin, or immunoglobulins; hydrophilic polymers such as polyvinylpyrolidone; amino acids such as glycine, glutamine, asparagine, histidine, arginine, or lysine; carbohydrates such as monosacchandes, disaccharides, glucose, mannose, or dextrins; chelating agents such as EDTA; sugars such as sucrose, mannitol, trehalose or sorbitol; salt-forming counter-ions such as sodium; metal complexes such as Zn-protein complexes; and/or non-ionic surfactants such as TWEEN or polyethylene glycol (PEG).

The pharmaceutical composition of the present invention can be administered in any number of ways for either local or systemic treatment. Administration can be topical (such as to mucous membranes including vaginal and rectal delivery) such as transdermal patches, ointments, lotions, creams, gels, drops, suppositories, sprays, liquids and powders; pulmonary such as by inhalation or insufflation of powders or aerosols (including by nebulizer), intratracheal, intranasal, epidermal and transdermal; oral; parenteral including intravenous, intraarterial, intratumoral, subcutaneous, intraperitoneal or intramuscular injection or infusion; or intracranial such as intrathecal or intraventricular.

The therapeutic formulation can be in unit dosage form. Such formulations include tablets, pills, capsules, powders, granules, solutions or suspensions in water or non-aqueous media, or suppositories for oral, parenteral, or rectal administration or for administration by inhalation. In solid compositions such as tablets the principal active ingredient is mixed with a pharmaceutical carrier. Conventional tableting ingredients include corn starch, lactose, sucrose, sorbitol, talc, stearic acid, magnesium stearate, dicalcium phosphate or gums, and other diluents (e.g., water) to form a solid preformulation composition containing a homogeneous mixture of a compound of the present invention, or a non-toxic pharmaceutically acceptable salt thereof. The solid preformulation composition is then subdivided into unit dosage forms of the type described herein. The tablets, pills, etc of the novel composition can be coated or otherwise compounded to provide a dosage form affording the advantage of prolonged action. For example, the tablet or pill can comprise an inner composition covered by an outer component. Furthermore, the two components can be separated by an enteric layer that serves to resist disintegration and permits the inner component to pass intact through the stomach or to be delayed in release. A variety of materials can be used for such enteric layers or coatings, such materials including a number of polymeric acids and mixtures of polymeric acids with such materials as shellac, cetyl alcohol and cellulose acetate.

Pharmaceutical formulations include antibodies of the present invention complexed with liposomes (Epstein, et al., 1985, PNAS, 82:3688; Hwang, et al., 1980, PNAS, 77:4030; and U.S. Pat. Nos. 4,485,045 and 4,544,545). Liposomes with enhanced circulation time are disclosed in U.S. Pat. No. 5,013,556. Some liposomes can be generated by the reverse phase evaporation with a lipid composition comprising phosphatidylcholine, cholesterol, and PEG-derivatized phosphatidylethanolamine (PEG-PE). Liposomes are extruded through filters of defined pore size to yield liposomes with the desired diameter.

The antibodies can also be entrapped in microcapsules. Such microcapsules are prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions as described in Remington: The Science and Practice of Pharmacy, 21^{st} Edition, University of the Sciences Philadelphia 2005.

In addition sustained-release preparations can be prepared. Suitable examples of sustained-release preparations include semi-permeable matrices of solid hydrophobic polymers containing the antibody, which matrices are in the form of shaped articles (e.g. films, or microcapsules). Examples of sustained-release matrices include polyesters, hydrogels such as poly(2-hydroxyethyl-methacrylate) or poly(vinylalcohol), polylactides (U.S. Pat. No. 3,773,919), copolymers of L-glutamic acid and 7 ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), sucrose acetate isobutyrate, and poly-D(-)-3-hydroxybutyric acid. In some embodiments the antibodies can be used to treat various conditions characterized by expression and/or increased responsiveness of cells to a cancer stem cell marker. Particularly it is envisioned that the antibodies against a cancer stem cell marker, for example NOTCH1, will be used to treat proliferative disorders including, but not limited to, benign and malignant tumors of the kidney, liver, bladder, breast, stomach, ovary, colon, rectum, prostate, lung, vulva, thyroid, head and neck, brain, and hematologic cancers such as leukemias and lymphomas. In certain embodiments, the hematologic cancer is chronic lymphocytic leukemia, hairy cell leukemia, chronic myelogenous leukemia, non-Hodgkin lymphoma, diffuse large B-cell lymphoma, mantle cell lymphoma, or cutaneous T-cell lymphoma.

In certain embodiments, in addition to administering a NOTCH1-binding antibody, the method or treatment further comprises administering at least one additional therapeutic agent or therapy. An additional therapeutic agent or therapy can be administered prior to, concurrently with, and/or subsequently to, administration of the NOTCH1-binding antibody. Pharmaceutical compositions comprising the NOTCH1-binding antibody and the additional therapeutic agent(s) are also provided. In some embodiments, the at least one additional therapeutic agent or therapy comprises 1, 2, 3, or more additional therapeutic agents or therapies.

Combination therapy with at least two therapeutic agents often uses agents that work by different mechanisms of action, although this is not required. Combination therapy using agents with different mechanisms of action may result in additive or synergetic effects. Combination therapy may allow for a lower dose of each agent than is used in monotherapy, thereby reducing toxic side effects. Combination therapy may decrease the likelihood that resistant cancer cells will develop. Combination therapy may allow for one agent to be targeted to tumorigenic cancer stem cells and a second agent to be targeted to non-tumorigenic cancer cells.

It will be appreciated that the combination of a NOTCH1-binding antibody and an additional therapeutic agent or therapy may be administered in any order or concurrently. In some embodiments, the NOTCH1-binding antibody will be administered to patients that have previously undergone treatment with a second therapeutic agent or therapy. In certain other embodiments, the NOTCH1-binding antibody and a second therapeutic agent or therapy will be administered substantially simultaneously or concurrently. For example, a subject may be given the NOTCH1-binding antibody while undergoing a course of treatment with a second therapeutic agent (e.g., chemotherapy). In certain embodiments, the NOTCH1-binding antibody will be administered within 1 year of the treatment with a second therapeutic agent. In certain alternative embodiments, the NOTCH1-binding antibody will be administered within 10, 8, 6, 4, or 2 months of any treatment with a second therapeutic agent. In certain other embodiments, the NOTCH1-binding antibody will be administered within 4, 3, 2, or 1 weeks of any treatment with a second therapeutic agent. In some embodiments, the NOTCH1-binding antibody will be administered within 5, 4, 3, 2, or 1 days of any treatment with a second therapeutic agent. It will further be appreciated that the two (or more) agents or treatments may be administered to the subject within a matter of hours or minutes (i.e., substantially simultaneously).

As is known to those of skill in the art, administration of any therapeutic agent may lead to side effects and/or toxicities. In some cases, the side effects and/or toxicities are so severe as to preclude administration of the particular agent at a therapeutically effective dose. In some cases, drug therapy must be discontinued, and other agents may be tried. However, many agents in the same therapeutic class often display similar side effects and/or toxicities, meaning that the patient either has to stop therapy, or if possible, suffer from the unpleasant side effects associated with the therapeutic agent.

Side effects from therapeutic agents may include, but are not limited to, hives, skin rashes, itching, nausea, vomiting, decreased appetite, diarrhea, chills, fever, fatigue, muscle aches and pain, headaches, low blood pressure, high blood pressure, hypokalemia, low blood counts, bleeding, and cardiac problems.

Thus, one aspect of the present invention is directed to methods of treating a hematologic cancer in a patient comprising administering an anti-NOTCH1 antibody using an intermittent dosing regimen, which may reduce side effects and/or toxicities associated with administration of the anti-NOTCH1 antibody. As used herein, “intermittent dosing” refers to a dosing regimen using a dosing interval of more than once a week, e.g., dosing once every 2 weeks, once every 3 weeks, once every 4 weeks, etc. In some embodiments, a method for treating a hematologic cancer in a human patient comprises administering to the patient an effective dose of an anti-NOTCH1 antibody according to an intermittent dosing regimen. In some embodiments, a method for treating a hematologic cancer in a human patient comprises administering to the patient an effective dose of an anti-NOTCH1 antibody according to an intermittent dosing regimen, and increasing the therapeutic index of the anti-NOTCH1 antibody. In some embodiments, the intermittent dosing regimen comprises administering an initial dose of an anti-NOTCH1 antibody to the patient, and administering subsequent doses of the anti-NOTCH1 antibody about once every 2 weeks. In some embodiments, the intermittent dosing regimen comprises administering an initial dose of an anti-NOTCH1 antibody to the patient, and administering subsequent doses of the anti-NOTCH1 antibody about once every 3 weeks. In some embodiments, the intermittent dosing regimen comprises administering an initial dose of an anti-NOTCH1 antibody to the patient, and administering subsequent doses of the anti-NOTCH1 antibody about once every 4 weeks.

In some embodiments, the subsequent doses in an intermittent dosing regimen are about the same amount or less than the initial dose. In other embodiments, the subsequent doses are a greater amount than the initial dose. As is known by those of skill in the art, doses used will vary depending on the clinical goals to be achieved. In some embodiments, the initial dose is about 0.25 mg/kg to about 20 mg/kg. In some embodiments, the initial dose is about 0.25, 0.5, 1, 2, 3, 4, 5, 6, 7, 8, 9, 10, 11, 12, 13, 14, 15, 16, 17, 18, 19, or 20 mg/kg. In certain embodiments, the initial dose is about 0.5 mg/kg. In certain embodiments, the initial dose is about 1 mg/kg. In certain embodiments, the initial dose is about 2.5 mg/kg. In certain embodiments, the initial dose is about 5 mg/kg. In certain embodiments, the initial dose is about 7.5 mg/kg. In certain embodiments, the initial dose is about 10 mg/kg. In certain embodiments, the initial dose is about 12.5 mg/kg. In certain embodiments, the initial dose is about 15 mg/kg. In certain embodiments, the initial dose is about 20 mg/kg. In some embodiments, the subsequent doses are about 0.25 mg/kg to about 15 mg/kg. In certain embodiments, the subsequent doses are about 0.5, 1, 2, 3, 4, 5, 6, 7, 8, 9, 10, 11, 12, 13, 14 or 15 mg/kg. In certain embodiments, the subsequent doses are about 0.5 mg/kg. In certain embodiments, the subsequent doses are about 1 mg/kg. In certain embodiments, the subsequent doses are about 2.5 mg/kg. In certain embodiments, the subsequent doses are about 5 mg/kg. In some embodiments, the subsequent doses are about 7.5 mg/kg. In some embodiments, the subsequent doses are about 10 mg/kg. In some embodiments, the subsequent doses are about 12.5 mg/kg.

In some embodiments, the intermittent dosing regimen comprises: (a) administering to the patient an initial dose of an anti-NOTCH1 antibody of about 2.5 mg/kg and (b) administering subsequent doses of about 2.5 mg/kg once every 2 weeks. In some embodiments, the intermittent dosing regimen comprises: (a) administering to the patient an initial dose of an anti-NOTCH1 antibody of about 5 mg/kg and (b) administering subsequent doses of about 5 mg/kg once every 2 weeks. In some embodiments, the intermittent dosing regimen comprises: (a) administering to the patient an initial dose of an anti-NOTCH1 antibody of about 2.5 mg/kg and (b) administering subsequent doses of about 2.5 mg/kg once every 3 weeks. In some embodiments, the intermittent dosing regimen comprises: (a) administering to the patient an initial dose of an anti-NOTCH1 antibody of about 5 mg/kg and (b) administering subsequent doses of about 5 mg/kg once every 3 weeks. In some embodiments, the intermittent dosing regimen comprises: (a) administering to the patient an initial dose of an anti-NOTCH1 antibody of about 2.5 mg/kg and (b) administering subsequent doses of about 2.5 mg/kg once every 4 weeks. In some embodiments, the intermittent dosing regimen comprises: (a) administering to the patient an initial dose of an anti-NOTCH1 antibody of about 5 mg/kg and (b) administering subsequent doses of about 5 mg/kg once every 4 weeks. In certain embodiments, the initial dose and the maintenance doses are different, for example, the initial dose is about 5 mg/kg and the subsequent doses are about 2.5 mg/kg. In certain embodiments, an intermittent dosing regimen may comprise a loading dose, for example, the initial dose is about 20 mg/kg and the subsequent doses are about 2.5 mg/kg or about 5 mg/kg administered once every 2 weeks, once every 3 weeks, or once every 4 weeks.

Another aspect of the present invention is directed to methods for reducing toxicity of an anti-NOTCH1 antibody in a human patient comprises administering to the patient the anti-NOTCH1 antibody using an intermittent dosing regimen. Another aspect of the present invention is directed to methods for reducing side effects of an anti-NOTCH1 antibody in a human patient comprises administering to the patient the anti-NOTCH1 antibody using an intermittent dosing regimen. Another aspect of the present invention is directed to methods for increasing the therapeutic index of an anti-NOTCH1 antibody in a human patient comprises administering to the patient the anti-NOTCH1 antibody using an intermittent dosing regimen.

The choice of delivery method for the initial and subsequent doses is made according to the ability of the animal or human patient to tolerate introduction of the anti-NOTCH1 antibody into the body. Thus, in any of the aspects and/or embodiments described herein, the administration of the anti-NOTCH1 antibody may be by intravenous injection or intravenously. In some embodiments, the administration is by intravenous infusion. In any of the aspects and/or embodiments described herein, the administration of the anti-NOTCH1 antibody may be by a non-intravenous route.

Therapeutic agents used for the treatment of hematologic cancers include, but are not limited to, antibiotics such as daunorubicin, doxorubicin, mitoxantrone and idarubicin; topoisomerase inhibitors such as etoposide, teniposide, and topotecan; DNA synthesis inhibitors such as carboplatin; DNA-damaging agents such as cyclophosphamide, bendamustine, chlorambucil, procarbazine, dacarbazine, and ifosfamide; cytotoxic enzymes such as asparaginase and pegaspargase; tyrosine kinases inhibitors such as imatinib mesylate, dasatinib, ponatinib, and nilotinib; antimetabolites such as azacitidine, clofarabine, cytarabine, cladribine, fludarabine, hydroxyurea, mercaptopurine, methotrexate, thioguanine, pralatrexate, and nelarabine; synthetic hormones such as prednisone, prednisolone and dexamethasone; antimitotic agents such as vincristine and vinblastine; monoclonal antibodies such as rituximab (e.g., RITUXAN), alemtuzumab, and ofatumumab; radioimmunotherapy agents such as Iodine I 131 tositumomab (e.g., BEXXAR) or ibritumomab tiuxetan (e.g., ZEVALIN); mTor inhibitors such as temsirolimus; histone deacetylase inhibitors such as vorinostat and romidepsin; hematopoietic stem cell mobilizers such as plerixafor; cytotoxic recombinant proteins such as denileukin difitox; protein synthesis inhibitors such as omacetaxine; immunomodulatory drugs such as thalidomide and lenalidomide; cyclin-dependent kinase inhibitors such as flavopiridol; and proteasome inhibitors such as bortezomib (e.g., VELCADE); as well as combinations thereof.

Therapeutic agents that may be administered in combination with the NOTCH1-binding antibodies include the above name therapeutic agents as well as other chemotherapeutic agents. Thus, in some embodiments, the method or treatment involves the combined administration of a NOTCH1-binding agent or antibody and a chemotherapeutic agent or cocktail of multiple different chemotherapeutic agents. Treatment with a NOTCH1-binding antibody can occur prior to, concurrently with, or subsequent to administration of chemotherapies. Combined administration can include co-administration, either in a single pharmaceutical formulation or using separate formulations, or consecutive administration in either order but generally within a time period such that all active agents can exert their biological activities simultaneously. Preparation and dosing schedules for such chemotherapeutic agents can be used according to manufacturers' instructions or as determined empirically by the skilled practitioner. Preparation and dosing schedules for such chemotherapy are also described in Chemotherapy Service Editor M. C. Perry, Williams & Wilkins, Baltimore, Md. (1992).

Chemotherapeutic agents useful in the instant invention include, but are not limited to, alkylating agents such as thiotepa and cyclophosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamime; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytosine arabinoside, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenishers such as folinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK; razoxane; sizofuran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (Ara-C); taxoids, e.g. paclitaxel and docetaxel; chlorambucil; gemcitabine; 6-thioguanine; mercaptopurine; platinum analogs such as cisplatin and carboplatin; vinblastine; platinum; etoposide; ifosfamide; mitomycin C; mitoxantrone; vincristine; vinorelbine; navelbine; novantrone; teniposide; daunomycin; aminopterin; xeloda; ibandronate; CPT11; topoisomerase inhibitor RFS 2000; difluoromethylornithine; retinoic acid; esperamicins; capecitabine; and pharmaceutically acceptable salts, acids or derivatives of any of the above. Chemotherapeutic agents also include anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above.

In certain embodiments, the chemotherapeutic agent is a topoisomerase inhibitor. Topoisomerase inhibitors are chemotherapy agents that interfere with the action of a topoisomerase enzyme (e.g., topoisomerase I or II). Topoisomerase inhibitors include, but are not limited to, doxorubicin HCl, daunorubicin citrate, mitoxantrone HIC1, actinomycin D, etoposide, topotecan HC, teniposide, and irinotecan, as well as pharmaceutically acceptable salts, acids, or derivatives of any of these.

In certain embodiments, the chemotherapeutic agent is an anti-metabolite. An anti-metabolite is a chemical with a structure that is similar to a metabolite required for normal biochemical reactions, yet different enough to interfere with one or more normal functions of cells, such as cell division. Anti-metabolites include, but are not limited to, gemcitabine, fluorouracil, capecitabine, methotrexate sodium, ralitrexed, pemetrexed, tegafur, cytosine arabinoside, thioguanine, 5-azacytidine, 6-mercaptopurine, azathioprine, 6-thioguanine, pentostatin, fludarabine phosphate, and cladribine, as well as pharmaceutically acceptable salts, acids, or derivatives of any of these.

In certain embodiments, the chemotherapeutic agent is an antimitotic agent, including, but not limited to, agents that bind tubulin. In some embodiments, the agent is a taxane. In certain embodiments, the agent is paclitaxel or docetaxel, or a pharmaceutically acceptable salt, acid, or derivative of paclitaxel or docetaxel. In certain alternative embodiments, the antimitotic agent comprises a vinca alkaloid, such as vincristine, binblastine, vinorelbine, or vindesine, or pharmaceutically acceptable salts, acids, or derivatives thereof.

In some embodiments, the treatment involves a NOTCH1-binding antibody described herein in combination with chemotherapeutic agent selected from the group consisting of prednisone, vincristine, daunorubicin, L-asparaginase, methotrexate and cyclophosphamide. In some embodiments, the additional therapeutic agent is imatinib, nelarabine or dastinib. In some embodiments, the additional therapeutic agent is idarubicin, cytosine arabinoside, mitoxantrone or gemtuzumab ozogamicin.

In certain embodiments, the treatment involves the combined administration of a NOTCH1-binding antibody of the present invention and radiation therapy. Treatment with the NOTCH1-binding antibody can occur prior to, concurrently with, or subsequent to administration of radiation therapy. Dosing schedules for such radiation therapy can be determined by the skilled medical practitioner. In some embodiments, the antibody is administered after radiation treatment. In some embodiments, the antibody is administered with radiation therapy.

In some embodiments, a second therapeutic agent comprises an antibody. Thus, treatment can involve the combined administration of a NOTCH1-binding antibody of the present invention with other antibodies against additional tumor-associated antigens including, but not limited to, antibodies that bind to EGFR, ErbB2, DLL4, NOTCH, or NF-κB. Exemplary anti-DLL4 antibodies are described, for example, in U.S. Pat. No. 7,750,124. Additional anti-DLL4 antibodies are described in, e.g., International Patent Pub. Nos. WO 2008/091222 and WO 2008/0793326, and U.S. Patent Application Pub. Nos. 2008/0014196; 2008/0175847; 2008/0181899; and 2008/0107648. Exemplary anti-NOTCH antibodies are described, for example, in U.S. Patent Application Pub. No. 2008/0131434. Combined administration can include co-administration, either in a single pharmaceutical formulation or using separate formulations, or consecutive administration in either order but generally within a time period such that all active agents can exert their biological activities simultaneously.

Furthermore, treatment with the NOTCH1-binding antibodies described herein can include combination treatment with one or more cytokines (e.g., lymphokines, interleukins, tumor necrosis factors, and/or growth factors) or can be accompanied by surgical removal of tumors, cancer cells or any other therapy deemed necessary by a treating physician. For the treatment of the disease, the appropriate dosage of an antibody of the present invention depends on the type of disease to be treated, the severity and course of the disease, the responsiveness of the disease, whether the antibody is administered for therapeutic or preventative purposes, previous therapy, patient's clinical history, and so on all at the discretion of the treating physician. The antibody can be administered one time or over a series of treatments lasting from several days to several months, or until a cure is effected or a diminution of the disease state is achieved (e.g. reduction in tumor size). Optimal dosing schedules can be calculated from measurements of drug accumulation in the body of the patient and will vary depending on the relative potency of an individual antagonist. The administering physician can easily determine optimum dosages, dosing methodologies and repetition rates. In general, dosage is from 0.01 μg to 100 mg per kg of body weight, and can be given once or more daily, weekly, monthly or yearly. The treating physician can estimate repetition rates for dosing based on measured residence times and concentrations of the antibody in bodily fluids or tissues.

### EXAMPLES

### Example 1

Antibodies were generated against a non-ligand binding region of NOTCH1, specifically the non-ligand binding membrane proximal region of the extracellular domain. In certain embodiments, recombinant polypeptide fragments of the human NOTCH1 extracellular domain were generated as antigens for antibody production. Standard recombinant DNA technology was used to isolate polynucleotides encoding the membrane proximal region of the extracellular domain of human NOTCH1 amino acids 1427-1732 (SEQ ID NO:1). These polynucleotides were separately ligated in-frame N-terminal to a human Fc and histidine-tag and cloned into a transfer plasmid vector for baculovirus-mediated expression in insect cells. Standard transfection, infection, and cell culture protocols were used to produce recombinant insect cells expressing the corresponding NOTCH1 polypeptide corresponding to a membrane proximal region comprising amino acids 1427-1732 (SEQ ID NO:2) (O'Reilly et al., 1994, Baculovirus Expression Vectors: A Laboratory Manual, Oxford: Oxford University Press).

NOTCH1 membrane proximal region (NOTCH1 amino acids 1472-1732) polypeptide was purified from insect cell lysates using protein A and Ni⁺⁺-chelate affinity chromatography as known to one skilled in the art. Purified NOTCH1 membrane proximal region polypeptide was dialyzed against PBS (pH=7), concentrated to approximately 1 mg/ml, and sterile filtered in preparation for immunization.

Mice (n=3) were immunized with purified NOTCH1 antigen protein (Antibody Solutions; Mountain View, Calif.) using standard techniques. Blood from individual mice was screened approximately 70 days after initial immunization for antigen recognition using ELISA and FACS analysis (described herein). The two animals with the highest antibody titers were selected for final antigen boost after which spleen cells were isolated for hybridoma production. Hybridoma cells were plated at 1 cell per well in 96 well plates, and the supernatant from each well screened by ELISA and FACS analysis against NOTCH1 membrane proximal region polypeptide. Several hybridomas with high antibody titer were selected and scaled up in static flask culture. Antibodies were purified from the hybridoma supernatant using protein A or protein G agarose chromatography. Purified monoclonal antibodies were tested again by FACS as described herein. Several antibodies that recognized the membrane proximal region of the extracellular domain of human NOTCH1 were isolated. A hybridoma cell line expressing antibody 52M51 was deposited with ATCC under the conditions of the Budapest Treaty on Aug. 7, 2008 and assigned ATTC Patent Deposit Designation PTA-9405. The nucleotide and predicted protein sequences of both the heavy chain (SEQ ID NO:9 and 10) and light chain (SEQ ID NO:3 and 4) of antibody 52M51 were determined.

### Human Antibodies

In alternative embodiments, human antibodies that specifically recognize the non-ligand binding membrane proximal region of the extracellular domain of a NOTCH1 receptor are isolated using phage display technology. In certain embodiments, a synthetic antibody library containing human antibody variable domains is screened for specific and high affinity recognition of a NOTCH receptor antigen described herein. In certain embodiments, a human Fab phage display library is screened using a series of recombinant proteins comprising the non-ligand binding membrane proximal region of the extracellular domain of a NOTCH 1 receptor. Briefly, 2×10¹³ Fab displaying phage particles are incubated with recombinant protein (passively immobilized) in round one, the non-specific phage are washed off, and then specific phage are eluted with either low pH (cells) or DTT (recombinant protein). The eluted output is used to infect TG1 F+ bacteria, rescued with helper phage, and then Fab display induced with IPTG (0.25 mM). This process is repeated for two additional rounds and then round three is screened in ELISA against passively immobilized antigen (5 μg/ml).

CDR cassettes in the library are specifically exchanged via unique flanking restriction sites for antibody optimization. Optimized human variable regions are then cloned into an Ig expression vector containing human IgG1 heavy-chain and kappa light-chain for expression of human antibodies in CHO cells.

### Epitope Mapping

To identify antibodies that recognize specific a non-ligand binding membrane proximal region of the NOTCH1 receptor extracellular domains, epitope mapping is performed. In certain embodiments, mammalian expression plasmid vectors comprising a CMV promoter upstream of polynucleotides that encode fragments of the extracellular NOTCH1 domain as Fc fusion proteins are generated using standard recombinant DNA technology. In certain embodiments, epitope mapping of the 52M series of non-ligand binding region antibodies is done using a series of fusion proteins and deletions of the membrane proximal region of the extracellular domain of a human NOTCH1 from about amino acid 1427 to about amino acid 1732. These recombinant fusion proteins are expressed in transiently transfected HEK 293 cells from which conditioned medium is collected twenty-four to forty-eight hours post-transfection for ELISA.

In certain embodiments, the NOTCH1 fusion protein fragments are separated on SDS-PAGE gels and probed with both anti-Fc antibodies to detect the presence of all fusion proteins versus anti-NOTCH1 antibodies to detect the domains recognized by each anti-NOTCH antibody.

### Example 2

Humanized antibodies against a membrane proximal region of the extracellular domain of a human NOTCH1 were generated. The variable domains of the murine monoclonal antibody 52M51 were isolated and sequenced from the hybridoma line using degenerate PCR essentially as described in Larrick, J. M., et al., 1989, Biochem. Biophys. Res. Comm. 160:1250 and Jones, S. T. & Bendig, M. M., 1991, Bio/Technology 9:88. Human heavy and light chain variable framework regions likely to be structurally similar to the parental 52M51 antibody amino acid sequences are then considered as reference human framework regions to help guide the design of novel synthetic frameworks. To identify the human framework regions bearing similarity to 52M51 murine frameworks, the predicted protein sequences encoded by the V_{H} and V_{L} murine variable domains of 52M51 are compared with human antibody sequences encoded by expressed human cDNA using BLAST searches for human sequence deposited in Genbank. Using this method, expressed human cDNA sequences (e.g., Genbank DA975021, DB242412) and germline Vh domains (e.g. IGHV1-24) were selected for further analysis in designing heavy chain frameworks. Similarly, expressed human cDNA sequences (e.g. Genbank CD709370, CD707373) and germline VI (e.g. IGLV7-46, IGLV8-61) were considered in designing light chain frameworks.

The amino acid differences between candidate humanized framework heavy chains and the parent murine monoclonal antibody 52M51 heavy chain variable domain and light chain variable domains were evaluated for likely importance, and a judgment made as to whether each difference in position contributes to proper folding and function of the variable domain. This analysis was guided by examination of solved crystal structures of other antibody fragments (e.g., the structure of Fab 2E8 as described in Trakhanov et al., 1999, Acta Crystallogr D Biol Crystallogr 55:122-28, as well as other protein crystal structures (e.g., protein data bank structures 1ADQ and 1GIG)). Structures were modeled using computer software including Jmol, quick PDB, and Pymol. Consideration was given to the potential impact of an amino acid at a given position on the packing of the β-sheet framework, the interaction between the heavy and light chain variable domains, the degree of solvent exposure of the amino acid side chain, and the likelihood that an amino acid would impact the positioning of the CDR loops. From this analysis, nine candidate V_{H} chains fused in-frame to the human IgG2 constant region and eight candidate VI chains fused in frame with the human IgLC1 constant region were conceived and chemically synthesized. The candidate heavy chains comprise: i) a synthetic framework designed to resemble natural human frameworks and ii) the parental 52M51 murine antibody CDRs.

The functionality of each candidate variant humanized heavy and light chain was tested by co-transfection into mammalian cells. Each of the nine candidate humanized 52M51 heavy chains described above was co-transfected into HEK 293 cells with the murine 52M51 light chain cDNA, and conditioned media was assayed by ELISA for NOTCH1-binding activity. The 52M51 heavy chain variant exhibiting the most robust binding was selected. This variant “52M51-H4” (SEQ ID NO:22) contains, in addition to murine CDRs, variation at 3 framework positions within the Vh framework, Kabat positions 20, 48, and 71 in comparison with an example human framework (e.g. IGHV1-24). The 52M51-H4 humanized heavy chain was then co-transfected into HEK293 cells with each of the eight candidate humanized light chains, and conditioned media was again assayed for antigen binding by ELISA. Two light chain variants “52M51-L3”(SEQ ID NO:26) and “52M51-L4” (SEQ ID NO:30) were found to exhibit better binding than the other candidates and were chosen for further study. Variant 52M51-L3 contains, in addition to murine CDRs, variation at 1 framework position at Kabat position 49 in comparison to an example human framework (e.g., IGLV7-46). Two humanized variant antibodies, 52M51-H4L3 and 52M51-H4L4, were developed. The polynucleotide encoding 52M51-H4L3 was deposited with the ATCC under the conditions of the Budapest Treaty on Oct. 15, 2008, and assigned designation number PTA-9549. Additional information about the murine 52M51 antibody, as well as the humanized variants 52M51-H4L3 and 52M51-1-4L4, is found in U.S. Patent Publication No. 2001/0311552, PCT Publication No. WO2010/005567, and PCT Publication No. WO2011/088215, each of which is hereby incorporated by reference herein in its entirety.

The affinities for human and mouse NOTCH11 were determined using a Biacore 2000 instrument. Briefly, recombinant human and mouse NOTCH1 proteins were immobilized on a CMS chip using standard amine based chemistry (NHS/EDC). Different antibody concentrations were injected over the protein surfaces and kinetic data were collected over time. The data was fit using the simultaneous global fit equation to yield dissociation constants (K_{D}, nM) for each NOTCH1 protein (Table 2).

TABLE 2
IgG Dissociation Constants (K_{D})

| Antibody | Human NOTCH1 (nM) | Mouse Notch1 (nM) |
|---|---|---|
| 52M51 | 2.86 | NB |
| 52M51-H4L3 | 4.33 | NB |
| 52M51-H4L4 | 7.35 | NB |

### Example 3

### NOTCH Receptor Signaling

The ability of NOTCH1 antibodies to block ligand-mediated NOTCH signaling was determined. HeLa cells engineered to over-express NOTCH1 (NOTCH1-HeLa) cultured in DMEM supplemented with antibiotics and 10% FCS were co-transfected with 1) pGL4 8×CBF firefly luciferase containing a NOTCH responsive promoter upstream of a firefly luciferase reporter gene to measure NOTCH signaling levels in response to DLL4 ligand; and 2) a Renilla luciferase reporter (Promega, Madison, Wis.) as an internal control for transfection efficiency. Transfected cells were added to cultures plates coated overnight with 200 ng/well of hDLL4-Fc protein, and antibodies to NOTCH1 were then added to the cell culture medium. Forty-eight hours following transfection, luciferase levels were measured using a dual luciferase assay kit (Promega, Madison Wis.) with firefly luciferase activity normalized to Renilla luciferase activity. The ability of antibodies to inhibit NOTCH1 pathway activation was thus determined. Antibodies 52M51, 52M63, 52M74, and 52M80, generated against a membrane proximal region of the extracellular domain of human NOTCH1 (FIG. 1A) significantly reduced luciferase activity indicative of reduced NOTCH1 signaling as compared to other NOTCH1 antibodies (FIG. 1B). Further, a humanized variant of antibody 52M51, variant 52M51-H4L3 displayed similar potency in reducing luciferase activity (FIG. 1C).

Cleavage of NOTCH receptors by furin, ADAM, and gamma-secretase results in formation of the NOTCH intracellular domain (ICD) that then triggers downstream NOTCH signaling in the nucleus. The ability of NOTCH1 antibodies to block ligand-mediated receptor activation was determined by Western blot analysis. NOTCH1-HeLa cells were grown in suspension culture in 293-SMII media (Invitrogen, Carlsbad Calif.). Cultured cells were transferred to 96-well plates in which select wells had been pre coated with human DLL4-Fc fusion protein (2 μg/ml) in DMEM plus 2% FBS and 1 μM MG132 (Calbiochem, San Diego Calif.). Antibodies generated against a membrane proximal region of the extracellular domain of human NOTCH1 were added to the cell culture medium, and cells were incubated at 37° C. for five hours. Wells were then aspirated and the cells resuspended in 2X SDS running buffer. Samples were sonicated at room temperature, and subjected to SDS-PAGE and Western blot analysis using an antibody specific for the cleaved NOTCH1 ICD according to the manufacturer's recommendations (Cell Signaling Technology, Danvers Mass.). Antibody 52M51 as well as antibodies 52M63, 52M74, and 52M80 significantly inhibited the generation of ICD after ligand stimulation (FIG. 1D).

### Example 4

### Identification of the L2482X NOTCH1 Mutation in DLBCL

DNA isolated from 30 chronic lymphoid leukemia (CLL) samples and 13 diffuse large B-cell lymphoma (DLBLC) samples was obtained. To detect the mutations in the portions of the NOTCH1 gene encoding the HD and PEST domains of NOTCH1, targeted sequencing of NOTCH1 (exon26, 27, 28, and 34) was performed using the Ion Torrent PGM sequencer (Life Technologies, Grand Island, N.Y.). Briefly, the genomic regions of the targeted NOTCH1 exons were PCR amplified and purified. The PCR products were pooled and a library was prepared according to Ion Torrent's standard protocol. The sequencing was performed on an Ion Torrent PGM sequencer and the sequences generated were mapped to UCSC human genome hg19 assembly using tMAPv1.5. After the removal of duplicate sequences, the nucleotide variations were called by Samtools (Li et al., 2009, Bioinformatics, 25:2078) and Varscan (Koboldt et al., 2009, Bioinformatics, 25:2283). The variant calls with <20× coverage, mapping quality <30 and variant calling quality <25 were omitted. The final variants were annotated by ANNOVAR (Wang et al, 2010, Nucleic Acids Research, 38:e164).

To validate the called variants, ˜250 bp amplicons spanning the mutated nucleotide sites were amplified by PCR and cloned using Topo-TA kit (Invitrogen/Life Technologies, Grand Island, N.Y.). 15-100 single clones were picked and DNA was isolated. The isolated DNA was sequenced using Sanger sequencing methods. The sequencing results were aligned to NOTCH1 Reference sequence NM_{—}017617 with Sequencher v4.10 (Gene Codes, Ann Arbor, Mich.).

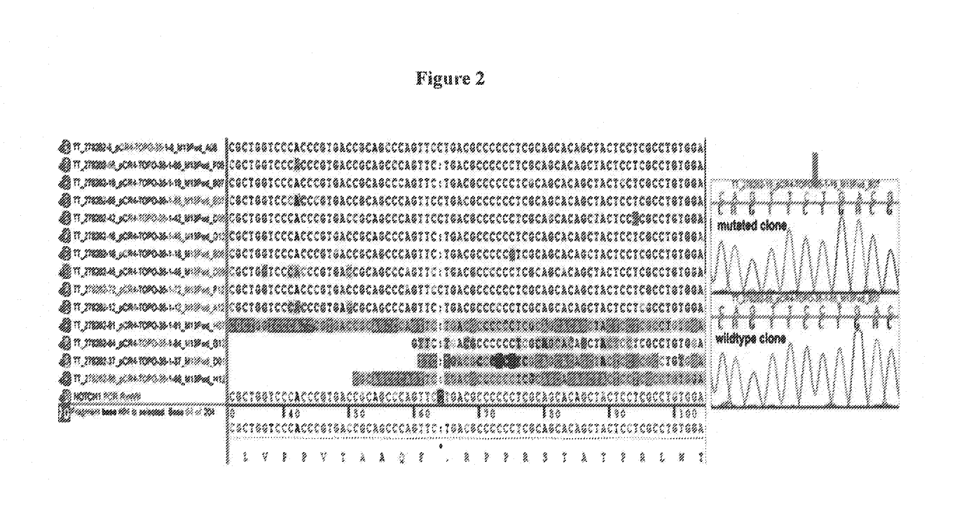

Five out of 30 CLL samples (17%) were reported to have an activating mutation in the NOTCH1 sequence encoding the PEST domain. One out of 13 DLBCL samples (13%) was identified to contain a novel mutation of the NOTCH1 PEST domain (wild-type NOTCH1 SEQ ID NO:34). Samtools and Varscan algorithms identified a heterozygous deletion of cytosine (C) at position 7444 (7444delC, p.L2482X, NM_{—}017617.3) in the NOTCH1 PEST domain in sample DLBCL-N020029. Visualization in the Integrative Genomics Viewer (IGV) showed the deletion of guanine (G) in the coding reverse strand in some sequence reads from this sample. The single cytosine deletion was validated by Sanger sequencing (FIG. 2). 10 of 15 single clones harbored the deletion. This mutation truncates the NOTCH1 PEST domain and is likely to be a NOTCH1 activating mutation.

### Example 5

### In Vitro Activity of DLBCL Mutant NOTCH1 Protein

A NOTCH1 construct containing the DLBCL mutation (deletion of cytosine at position 7444) was generated using Agilent QuikChange II XL Site-Directed Mutagenesis Kit (Agilent, La Jolla, Calif.). PCR primers were made using the Agilent primer design site. PCR reactions were run with 50 ng dsDNA NOTCH wild type template in pcDNA3.1 and other reaction ingredients per the protocol. Amplified DNA was digested with DpnI restriction enzyme and transformed using XL10-Gold Ultracompetent Cells. Colonies were selected and sequenced to confirm presence of the mutation. Full length sequencing was performed on the selected clones to ensure no additional mutations were present.

Human PC3 cells were transfected with an expression vector encoding a wild-type NOTCH1 or the NOTCH1 mutant protein described above, as well as plasmids encoding a NOTCH-dependent firefly luciferase reporter construct (8×CBS-luciferase) and a Renilla luciferase reporter (Promega, Madison, Wis.) as an internal control for transfection efficiency. Purified Jagged proteins were coated onto 96 well plates at 400 ng per well. After 24 hour incubation, transfected PC3 cells were collected, added to the wells, and incubated overnight. Luciferase activity was determined using the Dual-Glo luciferase assay kit (Promega, Madison, Wis.) with firefly luciferase activity normalized to Renilla luciferase activity.

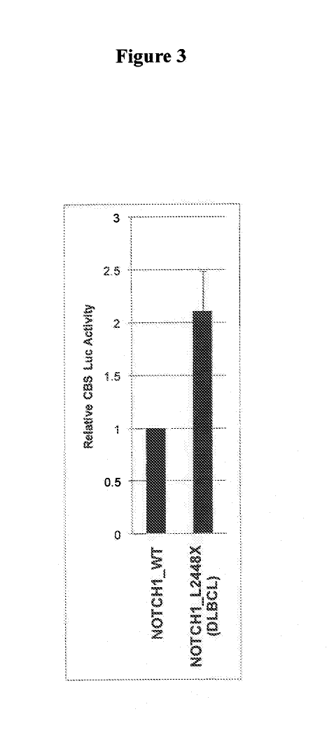

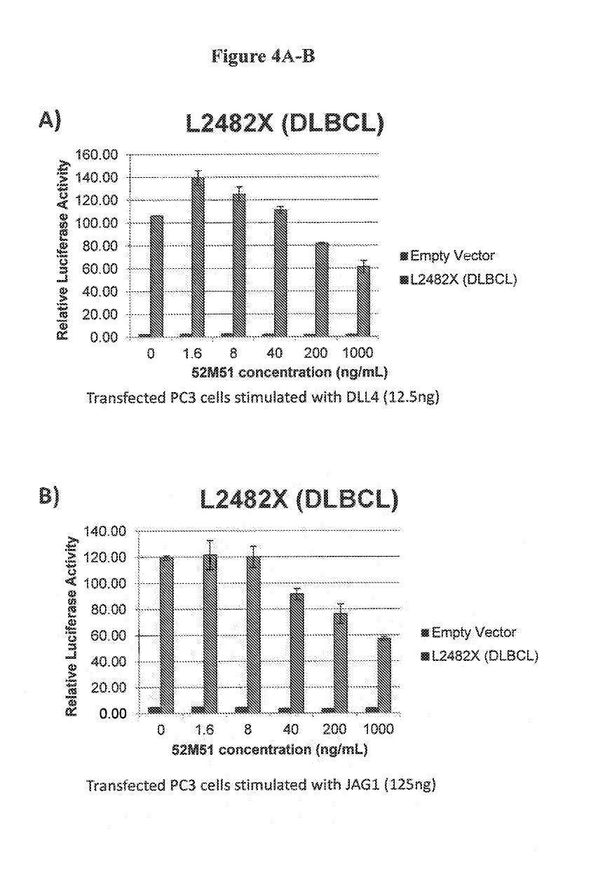

As shown in FIG. 3, the DLBCL NOTCH1 mutant protein has approximately 2-fold greater activity than the wild-type NOTCH1 protein, suggesting that the DLBCL NOTCH1 mutation is an activating mutation for NOTCH1 signaling.

### Example 6

### Development of CLL Xenograft Model

Four primary human CLL PBMC patient samples (LEU8, LEU9, LEU10, and LEU12), each harboring activating NOTCH 1 mutations (truncating mutations in the PEST domain), were used in a study to evaluate growth in vivo. Each sample was T-cell depleted using an anti-CD3 antibody conjugated to biotin, re-suspended in saline, and injected intravenously into the tail vein of ten 6-7 week old male NSG mice. Five of the ten mice were pre-conditioned with a sub-lethal dose of γ-irradiation (2.75 Gy) on the day of the injection. This was done to evaluate the requirement for pre-conditioning for in vivo engraftment. Whole body γ-irradiation was carried out in a Pantak HF320 X-ray machine.

The cell numbers injected per mouse for each sample were as follows: LEU8 (1.6×10⁵ cells/mouse), LEU9 (1×10⁶ cells/mouse), LEU10 (7×10⁴ cells/mouse), LEU12 (1.4×10⁶ cells/mouse). In addition, unfractionated LEU9 and LEU10 cells were injected intravenously into ten mice (five irradiated mice; five non-irradiated mice) each at 1×10⁶ cells per mouse. This was done alongside the previous groups to evaluate the requirement for autologous T-cells for engraftment.

Irradiated mice were maintained on antibiotic-containing water for up to one week prior and 2 weeks following irradiation. The antibiotics used were neomycin (1.1 mg/ml) and polymyxin B (110 ngm/l) with glucose 2 mg/ml. Mice were monitored twice weekly and body weights were taken weekly. Peripheral blood was collected from the submandibular vein two weeks after injection and analyzed for engraftment. Study endpoints include sacrifice of mice when >20% body weight loss and/or body conditioning score of <2 was observed. At sacrifice, spleen, bone marrow, liver and peripheral blood was collected and analyzed for level of human cell engraftment. Mouse lineage antibodies anti-CD45 and anti-H2K^{d} and human antibodies anti-CD19, anti-CD5, anti-CD45, anti-CD38 and anti-CD3 were used for detecting human cells by flow cytometry.

Based on the data at Day 65 post-injection, the results indicated that irradiation increases in vivo engraftment of the human CLL cells; 0-0.2% of human cells (mouse-lineage negative) were seen in the non-irradiated mice compared to 0.1-5% of human cells (mouse-lineage negative) in the irradiated mice. No difference was noted between the unfractionated and T-cell depleted groups, indicating that autologous T-cells do not improve engraftment in the context of activating NOTCH1 mutations. Five out of five mice from the LEU8 and two out of 4 mice from the LEU12 irradiated groups showed >10% decrease in body weights within one week (from Day 12 to Day 21) and were sacrificed on Day 22. At sacrifice, enlarged spleens were noted in all of the mice indicating that leukemic cells had engrafted in the mice. Flow cytometry analyses of the cells isolated from the spleens showed that 22.1% of the total cells were human cells (mouse-lineage negative) in the LEU8-engrafted mice and 18.9% of the total cells were human cells (mouse-lineage negative) in the LEU12-engrafted mice. 7.6% and 6.9% of the cells isolated from spleens stained positive for human marker CD19. The mice injected with the LEU9 and LEU10 CLL samples are continuing at day 65 and are being monitored weekly.

### Example 7

### Immunohistochemistry of Activated NOTCH1

A rabbit polyclonal antibody was developed which binds the cleavage site of NOTCH1 and specifically detects activated NOTCH1 (NOTCH1-ICD) within the nucleus.

NOTCH1-ICD immunohistochemistry (IHC) staining is performed with a tyramide signal amplification (TSA) modification of standard IHC protocol. Tissue samples are de-waxed and rehydrated then subjected to antigen retrieval in Dako TRS solution (Dako, Carpinteria, Calif.), under heat and pressure in a BioCare Decloaker benchtop pressure cooker. Alternatively, cells are attached to slides by standard methods, such as cytospin preparation. Endogenous peroxidase is blocked with 6% H₂O₂ in phosphate-buffered saline and the universal blocking agent CAS-Block is applied (Invitrogen/Life Technologies, Grand Island, N.Y.). Samples are incubated with primary anti-NOTCH1-ICD antibody overnight at 4° C. Sections are incubated with DAKO rabbit-HRP polymer (Dako, Carpinteria, Calif.), followed by FITC-labeled TSA substrate (Perkin Elmer, Waltham, Mass.). FITC is detected with HRP-conjugated anti-FITC antibodies (Rockland Immunochemicals, Gilbertsville, Pa.) and DAB substrate (Dako, Carpinteria, Calif.) added to visualize the antibody-detection complex.

The NOTCH1-ICD IHC assay may be used to monitor NOTCH pathway activity in hematologic cancers and pharmacodynamic response in treated subjects.

### Example 8

### Phase 1 Study

An open-label Phase 1 dose escalation study of anti-NOTCH1 antibody 52M51-H4L3 (also referred to as OMP-52M51) in patients with previously treated hematologic cancers is in the process of being initiated. The unselected patient population will include patients with relapsed and/or refractory CLL, MCL, DLBCL, mycosis fungoides, and Sézary syndrome. Prior to enrollment, patients will undergo screening to determine study eligibility. Samples from patients will be tested for NOTCH1 mutations. The study endpoints include the determination of the safety profile, pharmacokinetics (PK), pharmacodynamics (PD), preliminary efficacy, and to determine maximum tolerated dose (MTD). In the initial phase of the study, dose escalation is performed to determine the maximum tolerated dose of OMP-52M51. The drug is administered intravenously once every 4 weeks at dose levels of 0.25, 0.5, 1.0, 2.5, 5, and 10 mg/kg until disease progression or unacceptable tolerability. No dose escalation or reduction is allowed within a dose cohort. Three patients are treated at each dose level if no dose-limiting toxicities (DLTs) are observed. If 1 of 3 patients experience a DLT, the dose level is expanded to 6 patients. If 2 or more patients experience a DLT, no further patients are dosed at that level and 3 additional patients are added to the preceding dose cohort unless 6 patients are being treated at that dose level. Patients are assessed for DLTs for 28 days after the administration of the first dose of OMP-52M51. The MTD is defined as the highest dose level that resulted in less than 2 of 6 subjects experiencing a DLT.

After MTD is defined, an expansion cohort (n=20) is to be added to the study. The expansion cohort will be a patient population selected for CLL, MCL, and DLBCL cancers with a NOTCH1 mutation, and patients with mycosis fungoides and Sézary syndrome (no NOTCH1 mutation required). In addition, if signs of drug activity in the dose escalation phase in patients that do not have a NOTCH1 mutation are observed, a second expansion cohort including patients with these cancers (e.g., CLL, MCL, DLBCL) without a NOTCH1 mutation may be added to the study.

### Example 9

### NOTCH1 Mutation Assay

Patient samples will be tested for NOTCH1 mutations in the HD and PEST domains using a next generation sequencing assay (NGS). The NOTCH1 exons to be sequenced at a depth of 500× (after all duplications have been removed) are exon 26 (nt 4587-5018 in reference sequence NM_{—}017617) in the HD domain, exon 27 and 28 (nt 5019-5384) in the HD domain, and exon 34 (nt 6181-7668) in the PEST domain. Alternatively, patient samples will be tested for NOTCH1 mutations using Sanger sequencing of exon 34 in the PEST domain.

### Example 10

52M51 anti-NOTCH1 antibody inhibits ligand mediated signaling by the L2482X and P2514fs mutant NOTCH1 polypeptide.

In certain embodiments, the ability of the 52M51 NOTCH1 receptor antibody to block ligand-mediated signaling by a L2482X and P2514fs (Wang et al., N. Engl. J. Med (2011) 365:2497-506) mutant NOTCH1 polypeptide was determined. In certain embodiments, PC3 cells were co-transfected with (a) a vector expressing L2482X, P2514fs, or wild type NOTCH1, (b) the pGL4_{—}8×CBS vector comprising a Notch responsive promoter upstream of a firefly luciferase reporter gene, (c) pcDNA3_Mammal and (d) the pGL3_RL.CMV vector expressing Renilla luciferase. Control cells were transfected with an empty vector in place of (a). DNA transfection was carried out using OptiMEM and FuGENE 6. Transfection reagents were mixed and incubated at room temperature for 15 minutes before being added to the cells. Transfected PC3 cells were incubated overnight at 37° C./5% CO₂. At the time of adding the transfection reagents to the cells, 96-well plates were coated with hDLL4 (12.5 ng) or hJAG1 (125 ng) (R&D Systems; Minneapolis, Minn.) or no ligand in 30 μl PBS per well. Coated plates were stored overnight at 4′C. After 24 hour transient transfection, cells were collected and 70 μl/well were added to the 96-well coated plates before incubating overnight at 37° C./5% CO₂. At least 1 hour prior to addition of the transfected cells, 10 ul/well (1.6-1000 ng/ml) 52M51 anti-NOTCH1 antibody was added into the 96 well plates. NOTCH activity was assessed using the Dual-Glo luciferase Assay System (Promega; Madison, Wis.). NOTCH activity was calculated taking the ratio of Firefly luciferase to Renilla luciferase.

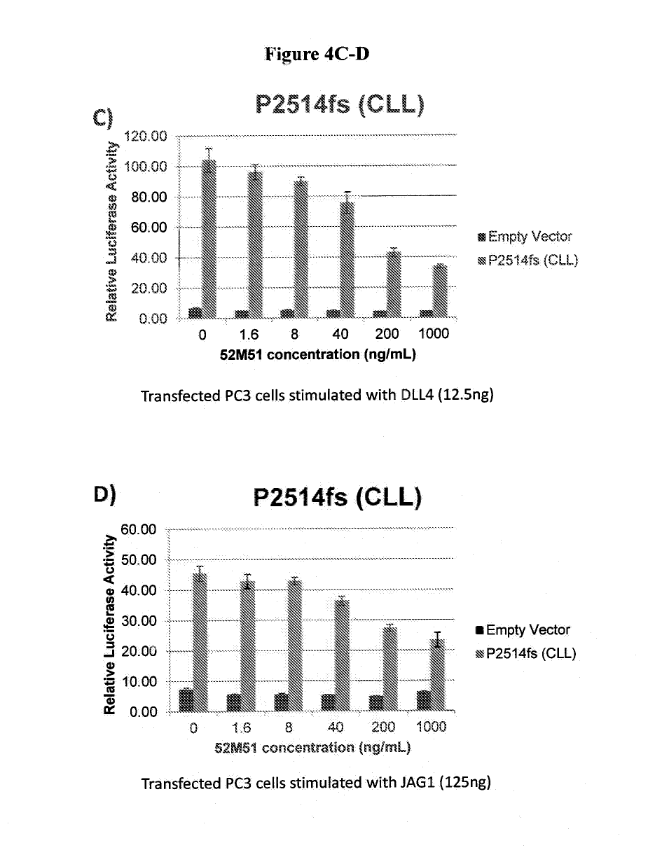

FIGS. 4A and B show the Firefly luciferase to Renilla luciferase activity ratio observed in L2482X NOTCH1 mutant polypeptide expressing PC3 cells after stimulation with DLL4 and JAG1, respectively. FIGS. 4C and D show the Firefly luciferase to Renilla luciferase activity ratio observed in P2514fs NOTCH1 mutant polypeptide expressing PC3 cells after stimulation with DLL4 and JAG1, respectively. PC3 cells expressing L2482X or P2514fs NOTCH1 had significantly higher Firefly luciferase to Renilla luciferase activity ratio than cells not expressing a recombinant NOTCH1. Further, the 52M51 anti-NOTCH1 antibody reduced the L2482X and P2514fs NOTCH1-mediated increase in Firefly luciferase to Renilla luciferase activity ratio in a dose dependent manner.

### Example 11

### Identification of the P1390T NOTCH1 Mutation in DLBCL

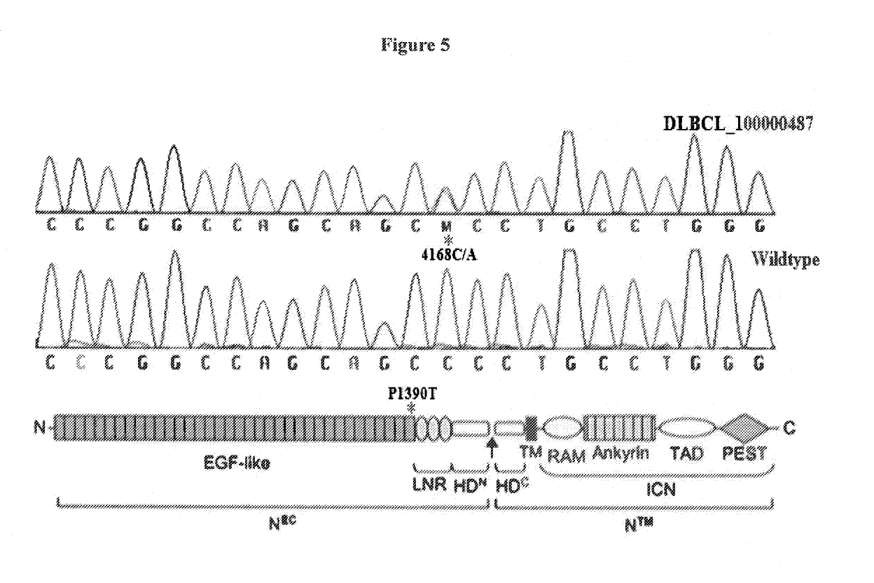

A heterozygous missense mutation (C4168A, p.P1390T, NM_{—}017617.3) was identified in a DLBCL tumor sample (DLBCL_{—}10000487) (FIG. 5). This mutation resides in NOTCH1 exon 25 and the last EGF-like domain, i.e., EGF domain 36. The C4168A missense mutation results in a proline to threonine substitution at amino acid position 1390 (P1390T). This mutation was predicted to be damaging using the PolyPhen-2 sequence analysis software.

### Example 12

### In Vitro Treatment of NOTCH1 Mutant Mantle Cell Lymphoma Cell Line and Primary NOTCH1 Mutant CLL Cells with 52M51 Anti-NOTCH1 Antibody

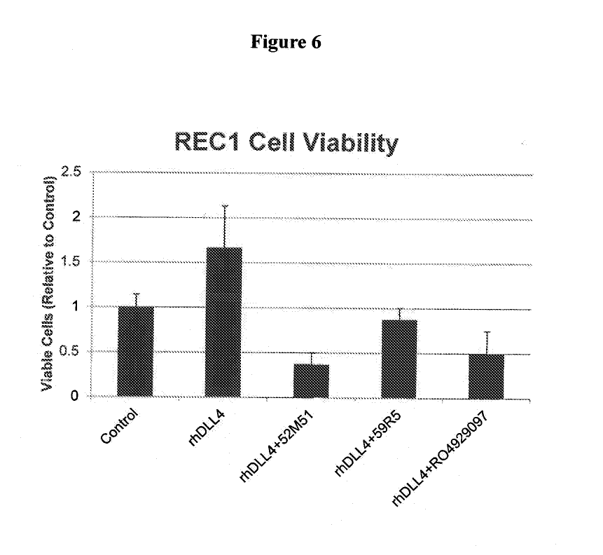

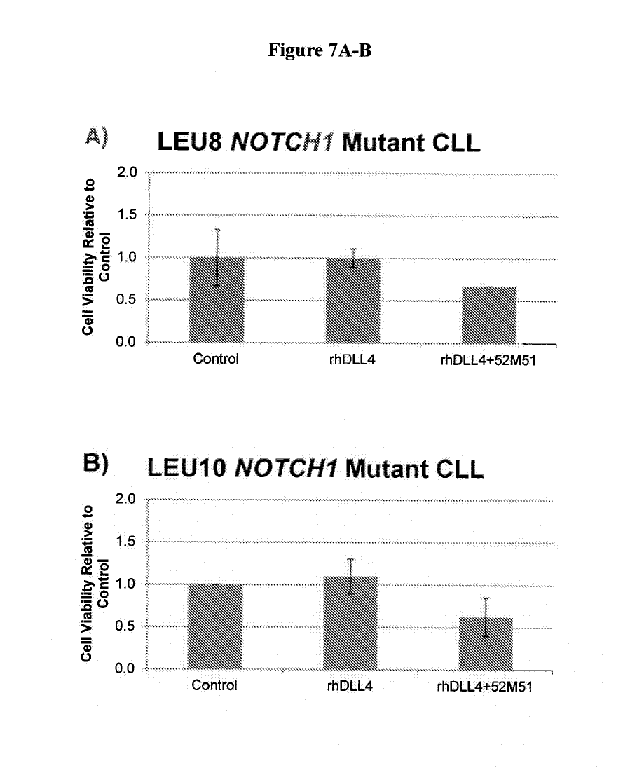

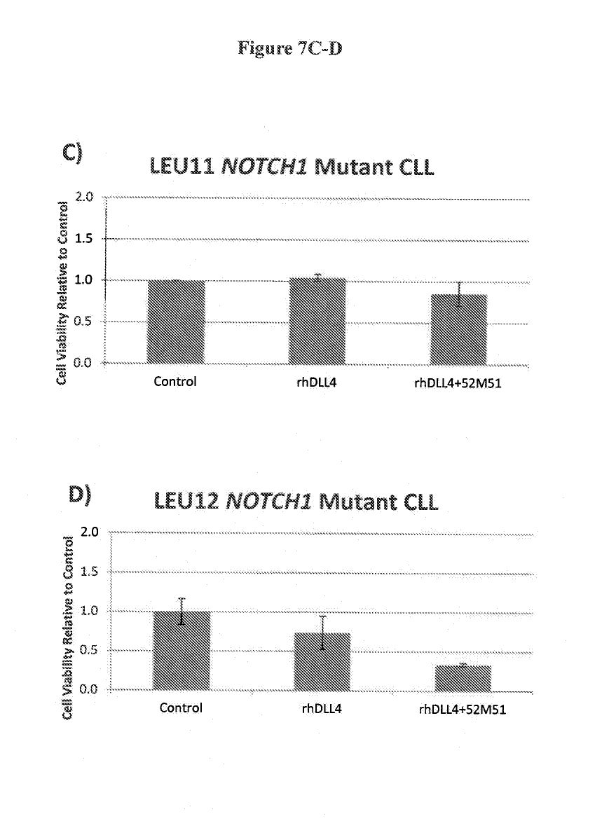

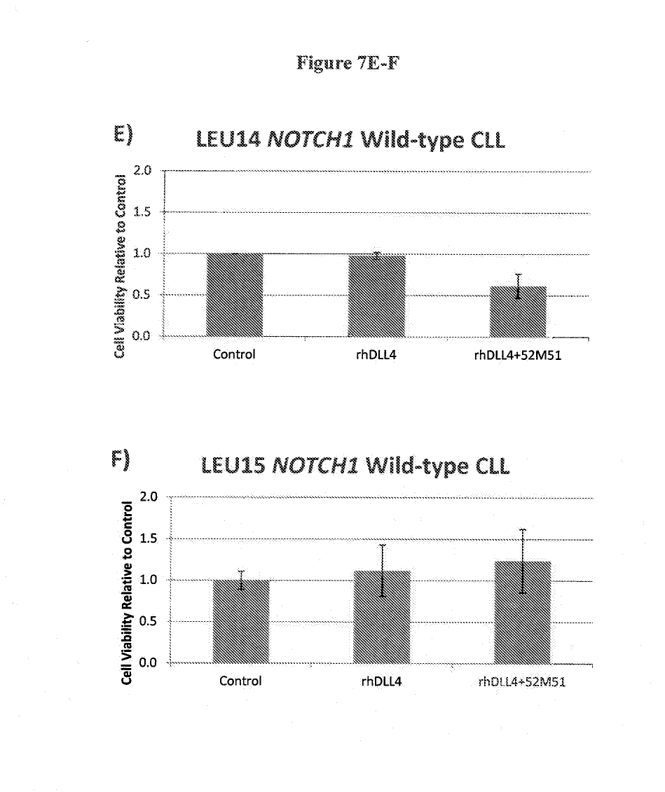

The extent to which 52M51 anti-NOTCH1 antibody treatment blocks the effects of NOTCH1 activation in NOTCH1 wild-type and NOTCH1ΔPEST mutant B cell malignancies was determined. One microgram of recombinant human DLL4 (R&D Systems) in 250 μl PBS was plated onto each well of a TC-treated 24-well plate by incubation at 4° C. for 4-6 hours. Control wells were incubated with 250 μl PBS. Subsequently, approximately one million primary CLL cells or thrice PBS-washed REC1 mantle cell lymphoma cells were plated in the presence of 25 μg/ml 52M51 anti-NOTCH1 antibody, 25 μg/ml 59R5 anti-NOTCH2/3 antibody (e.g., U.S. Pat. No. 8,226,943), or 10M RO4929097 gamma-secretase inhibitor. Primary CLL cells and REC1 cells were harvested 24 hours and 40 hours, respectively, after culture initiation and viable cells were counted by trypan blue exclusion (FIGS. 6 and 7).

Cells from four independent NOTCH1ΔPEST mutant CLL samples, two NOTCH1 wild-type CLL samples, and the REC1 NOTCH1ΔPEST mantle cell lymphoma cell line were stimulated with recombinant human DLL4 (rhDLL4) in the presence the 52M51 anti-NOTCH1 antibody. The number of viable CLL cells did not significantly change in response to rhDLL4 (FIG. 6 A-F). However, the number of viable cells was reduced in all four NOTCH1ΔPEST mutant CLL samples (LEU8, LEU10, LEU11 and LEU12 shown in FIG. 7 A-D) and in one of the two NOTCH1 wild-type CLL samples (LEU14 shown in FIG. 7E) when cultured in the presence of the 52M51 anti-NOTCH1 antibody and rhDLL4 for 24 hours. The number of viable REC1 cells increased following culture for 40 hours in the presence of rhDLL4 (FIG. 6). Treatment with the 52M51 anti-NOTCH1 antibody or gamma secretase inhibitor RO4929097, but not with the 59R5 anti-NOTCH2/3 antibody, suppressed the number of viable REC1 cells below levels seen in control cultures not stimulated with rhDLL4.

It is understood that the examples and embodiments described herein are for illustrative purposes only and that various modifications or changes in light thereof will be suggested to persons skilled in the art and are to be included within the spirit and purview of this application.

All publications, patents and patent applications cited herein are hereby incorporated by reference in their entirety for all purposes to the same extent as if each individual publication, patent or patent application were specifically and individually indicated to be so incorporated by reference.

SEQUENCES

SEQ ID NO: 1 NOTCH1 Nucleic acid sequence encoding amino acids 1427-1732.
CACATCCTGGACTACAGCTTCGGGGGTGGGGCCGGGCGCGACATCCCCCCGCCGCTGATC
GAGGAGGCGTGCGAGCTGCCCGAGTGCCAGGAGGACGCGGGCAACAAGGTCTGCAGCCTG
CAGTGCAACAACCACGCGTGCGGCTGGGACGGCGGTGACTGCTCCCTCAACTTCAATGAC
CCCTGGAAGAACTGCACGCAGTCTCTGCAGTGCTGGAAGTACTTCAGTGACGGCCACTGT
GACAGCCAGTGCAACTCAGCCGGCTGCCTCTTCGACGGCTTTGACTGCCAGCGTGCGGAA
GGCCAGTGCAACCCCCTGTACGACCAGTACTGCAAGGACCACTTCAGCGACGGGCACTGC
GACCAGGGCTGCAACAGCGCGGAGTGCGAGTGGGACGGGCTGGACTGTGCGGAGCATGTA
CCCGAGAGGCTGGCGGCCGGCACGCTGGTGGTGGTGGTGCTGATGCCGCCGGAGCAGCTG
CGCAACAGCTCCTTCCACTTCCTGCGGGAGCTCAGCCGCGTGCTGCACACCAACGTGGTC
TTCAAGCGTGACGCACACGGCCAGCAGATGATCTTCCCCTACTACGGCCGCGAGGAGGAG
CTGCGCAAGCACCCCATCAAGCGTGCCGCCGAGGGCTGGGCCGCACCTGACGCCCTGCTG
GGCCAGGTGAAGGCCTCGCTGCTCCCTGGTGGCAGCGAGGGTGGGCGGCGGCGGAGGGAG
CTGGACCCCATGGACGTCCGCGGCTCCATCGTCTACCTGGAGATTGACAACCGGCAGTGT
GTGCAGGCCTCCTCGCAGTGCTTCCAGAGTGCCACCGACGTGGCCGCATTCCTGGGAGCG
CTCGCCTCGCTGGGCAGCCTCAACATCCCCTACAAGATCGAGGCCGTGCAGAGTGAGACC
GTGGAGCCGCCCCCGCCG
SEQ ID NO: 2 NOTCH1 amino acids 1427-1732
HILDYSFGGGAGRDIPPPLIEEACELPECQEDAGNKVCSLQCNNHACGWDGGDCSLNFND
PWKNCTQSLQCWKYFSDGHCDSQCNSAGCLFDGFDCQRAEGQCNPLYDQYCKDHFSDGHC
DQGCNSAECEWDGLDCAEHVPERLAAGTLVVVVLMPPEQLRNSSFHFLRELSRVLHTNVV
FKRDAHGQQMIFPYYGREEELRKHPIKRAAEGWAAPDALLGQVKASLLPGGSEGGRRRRE
LDPMDVRGSIVYLEIDNRQCVQASSQCFQSATDVAAFLGALASLGSLNIPYKIEAVQSET
VEPPPP
Mouse antibody 52M51 sequences
SEQ ID NO: 3 52M51 Light chain polynucleotide sequence (Putative
signal sequence underlined)
ATGGCCTGGATTTCACTTATACTCTCTCTCCTGGCTCTCAGCTCAGGGGCCATTTCCCAG
GCTGTTGTGACTCAGGAATCTGCACTCACCACATCACCTGGTGAAACAGTCACACTCACT
TGTCGCTCAAGTACTGGGGCTGTTACAACTAGTAACTACGCCAACTGGGTCCAAGAAAAA
CCTGATCATTTATTCACTGGTCTAATAGGTGGTACCAACAACCGAGCTCCAGGTGTTCCT
GCCAGATTCTCAGGCTCCCTGATTGGAGACAAGGCTGCCCTCACCATCACAGGGGCACAG
ACTGAGGATGAGGCAATATATTTCTGTGCTCTATGGTACAGCAACCACTGGGTGTTCGGT
GGAGGAACCAAACTGACTGTCCTAGGCCAGCCCAAGTCTTCGCCATCAGTCACCCTGTTT
CCACCTTCCTCTGAAGAGCTCGAGACTAACAAGGCCACACTGGTGTGTACGATCACTGAT
TTCTACCCAGGTGTGGTGACAGTGGACTGGAAGGTAGATGGTACCCCTGTCACTCAGGGT
ATGGAGACAACCCAGCCTTCCAAACAGAGCAACAACAAGTACATGGCTAGCAGCTACCTG
ACCCTGACAGCAAGAGCATGGGAAAGGCATAGCAGTTACAGCTGCCAGGTCACTCATGAA
GGTCACACTGTGGAGAAGAGTTTGTCCCGTGCTGACTGTTCCTAG
SEQ ID NO: 4 52M51 Light chain amino acid sequence (Putative
signal sequence underlined)
MAWISLILSLLALSSGAISQAVVTQESALTTSPGETVTLTCRSSTGAVTTSNYANWVQEK
PDHLFTGLIGGTNNRAPGVPARFSGSLIGDKAALTITGAQTEDEAIYFCALWYSNHWVFG
GGTKLTVLGQPKSSPSVTLFPPSSEELETNKATLVCTITDFYPGVVTVDWKVDGTPVTQG
METTQPSKQSNNKYMASSYLTLTARAWERHSSYSCQVTHEGHTVEKSLSRADCS
SEQ ID NO: 5
52M51 Light chain variable region polynucleotide sequence (Putative
signal sequence underlined)
ATGGCCTGGATTTCACTTATACTCTCTCTCCTGGCTCTCAGCTCAGGGGCCATTTCCCAG
GCTGTTGTGACTCAGGAATCTGCACTCACCACATCACCTGGTGAAACAGTCACACTCACT
TGTCGCTCAAGTACTGGGGCTGTTACAACTAGTAACTACGCCAACTGGGTCCAAGAAAAA
CCTGATCATTTATTCACTGGTCTAATAGGTGGTACCAACAACCGAGCTCCAGGTGTTCCT
GCCAGATTCTCAGGCTCCCTGATTGGAGACAAGGCTGCCCTCACCATCACAGGGGCACAG
ACTGAGGATGAGGCAATATATTTCTGTGCTCTATGGTACAGCAACCACTGGGTGTTCGGT
GGAGGAACCAAACTGACTGTCCTAGGC
SEQ ID NO: 6 52M51 Light chain variable region amino acid sequence
(Putative signal sequence underlined)
MAWISLILSLLALSSGAISQAVVTQESALTTSPGETVTLTCRSSTGAVTTSNYANWVQEK
PDHLFTGLIGGTNNRAPGVPARFSGSLIGDKAALTITGAQTEDEAIYFCALWYSNHWVFG
GGTKLTVLGQPKSSPSVTLFPPSSEELETNKATLVCTITDFYPGVVTVDWKVDGTPVTQG
SEQ ID NO: 7 52M5 Light chain variable region polynucleotide sequence
without putative signal sequence
CAGGCTGTTGTGACTCAGGAATCTGCACTCACCACATCACCTGGTGAAACAGTCACACTC
ACTTGTCGCTCAAGTACTGGGGCTGTTACAACTAGTAACTACGCCAACTGGGTCCAAGAA
AAACCTGATCATTTATTCACTGGTCTAATAGGTGGTACCAACAACCGAGCTCCAGGTGTT
CCTGCCAGATTCTCAGGCTCCCTGATTGGAGACAAGGCTGCCCTCACCATCACAGGGGCA
CAGACTGAGGATGAGGCAATATATTTCTGTGCTCTATGGTACAGCAACCACTGGGTGTTC
GGTGGAGGAACCAAACTGACTGTCCTAGGC
SEQ ID NO: 8 52M51 Light chain variable region amino acid sequence
without putative signal sequence
QAVVTQESALTTSPGETVTLTCRSSTGAVTTSNYANWVQEKPDHLFTGLIGGTNNRAPGV
PARFSGSLIGDKAALTITGAQTEDEAIYFCALWYSNHWVFGGGTKLTVLG
SEQ ID NO: 9 52M51 Heavy chain polynucleotide sequence (Putative
signal sequence underlined)
ATGGAATGGACCTGGGTCTTTCTCTTCCTCCTGTCAGTAACTGCAGGTGTCCACTCCCAG
GTTCAGCTGCAGCAGTCTGGAGCTGAGCTGATGAAGCCTGGGGCCTCAGTGAAGATATCC
TGCAAGGCTGCTGGCTACACAATGAGAGGCTACTGGATAGAGTGGATAAAGCAGAGGCCT
GGACATGGCCTTGAGTGGATTGGACAGATTTTACCTGGAACTGGGAGAACTAACTACAAT
GAGAAGTTCAAGGGCAAGGCCACATTCACTGCAGATACATCCTCCAACACAGCCAACATG
CAACTCAGCAGCCTGACATCTGAGGACTCTGCCGTCTATTACTGTGCAAGATTTGATGGT
AACTACGGTTACTATGCTATGGACTACTGGGGTCAAGGATCCTCAGTCACCGTCTCCTCA
GCCAAAACGACACCCCCATCTGTCTATCCACTGGCCCCTGGATCTGCTGCCCAAACTAAC
TCCATGGTGACCCTGGGATGCCTGGTCAAGGGCTATTTCCCTGAGCCAGTGACAGTGACC
TGGAACTCTGGATCCCTGTCCAGCGGTGTGCACACCTTCCCAGCTGTCCTGCAGTCTGAC
CTCTACACTCTGAGCAGCTCAGTGACTGTCCCCTCCAGCCCTCGGCCCAGCGAGACCGTC
ACCTGCAACGTTGCCCACCCGGCCAGCAGCACCAAGGTGGACAAGAAAATTGTGCCCAGG
GATTGTGGTTGTAAGCCTTGCATATGTACAGTCCCAGAAGTATCATCTGTCTTCATCTTC
CCCCCAAAGCCCAAGGATGTCCTCACCATTACTCTGACTCCTAAGGTCACGTGTGTTGTG
GTAGACATCAGCAAGGATGATCCCGAGGTCCAGTTCAGCTGGTTTGTAGATGATGTGGAG
GTGCACACAGCTCAGACGCAACCCCGGGAGGAGCAGTTCAACAGCACTTTCCGCTCAGTC
AGTGAACTTCCCATCATGCACCAGGACTGGCTCAATGGCAAGGAGTTCAAATGCAGGGTC
AACAGTGCAGCTTTCCCTGCCCCCATCGAGAAAACCATATCCAAAACCAAAGGCAGACCG
AAGGCTCCACAGGTGTACACCATTCCACCTCCCAAGGAGCAGATGGCCAAGGATAAAGTC
AGTCTGACCTGCATGATAACAGACTTCTTCCCTGAAGACATAACAGTGGAGTGGCAGTGG
AATGGGCAGCCAGCGGAGAACTACAAGAACACTCAGCCCATCATGAACACGAATGGCTCT
TACTTCGTCTACAGCAAGCTCAATGTGCAGAAGAGCAACTGGGAGGCAGGAAATACTTTC
ACCTGCTCTGTGTTACATGAGGGCCTGCACAACCACCATACTGAGAAGAGCCTCTCCCAC
TCTCCTGGTAAATGA
SEQ ID NO: 10 52M51 Heavy chain amino acid sequence (Putative
signal sequence underlined)
MEWTWVFLFLLSVTAGVHSQVQLQQSGAELMKPGASVKTSCKAAGYTMRGYWIEWIKQRP
GHGLEWIGQILPGTGRTNYNEKFKGKATFTADTSSNTANMQLSSLTSEDSAVYYCARFDG
NYGYYAMDYWGQGSSVTVSSAKTTPPSVYPLAPGSAAQTNSMVTLGCLVKGYFPEPVTVT
WNSGSLSSGVHTFPAVLQSDLYTLSSSVTVPSSPRPSETVTCNVAHPASSTKVDKKIVPR
DCGCKPCICTVPEVSSVFIFPPKPKDVLTITLTPKVTCVVVDISKDDPEVQFSWFVDDVE
VHTAQTQPREEQFNSTFRSVSELPIMHQDWLNGKEFKCRVNSAAFPAPIEKTISKTKGRP
KAPQVYTIPPPKEQMAKDKVSLTCMITDFFPEDITVEWQWNGQPAENYKNTQPIMNTNGS
YFVYSKLNVQKSNWEAGNTFTCSVLHEGLHNHHTEKSLSHSPGK
SEQ ID NO: 11 52M51 Heavy chain variable region polynucleotide
sequence (Putative signal sequence underlined)
ATGGAATGGACCTGGGTCTTTCTCTTCCTCCTGTCAGTAACTGCAGGTGTCCACTCCCAG
GTTCAGCTGCAGCAGTCTGGAGCTGAGCTGATGAAGCCTGGGGCCTCAGTGAAGATATCC
TGCAAGGCTGCTGGCTACACAATGAGAGGCTACTGGATAGAGTGGATAAAGCAGAGGCCT
GGACATGGCCTTGAGTGGATTGGACAGATTTTACCTGGAACTGGGAGAACTAACTACAAT
GAGAAGTTCAAGGGCAAGGCCACATTCACTGCAGATACATCCTCCAACACAGCCAACATG
CAACTCACCAGCCTGACATCTGAGGACTCTGCCGTCTATTACTGTGCAAGATTTGATGGT
AACTACGGTTACTATGCTATGGACTACTGGGGTCAAGGATCCTCAGTCACCGTCTCCTCA
SEQ ID NO: 12 52M51 Heavy chain variable region amino acid sequence
(Putative signal sequence underlined)
MEWTWVFLFLLSVTAGVHSQVQLQQSGAELMKPGASVKISCKAAGYTMRGYWIEWIKQRP
GHGLEWIGQILPGTGRTNYNIKFKGKATFTADTSSNTANMQLSSLTSEDSAVYYCARFDG
NYGYYANDYWGQGSSVTVSSAKTTPPSVYPLAPGSAAQTNSMVTLGCLVKGYIPEPVTVT
SEQ ID NO: 13 52M51 Heavy chain variable region polynucleotide sequence
without putative signal sequence
CAGGTTCAGCTGCAGCAGTCTGGAGCTGAGCTGATGAAGCCTGGGGCCTCAGTGAAGATA
TCCTGCAAGGCTGCTGGCTACACAATGAGAGGCTACTGGATAGAGTGGATAAAGCAGAGG
CCTGGACATGGCCTTGAGTGGATTGGACAGATTTTACCTGGAACTGGGAGAACTAACTAC
AATGAGAAGTTCAAGGGCAAGGCCACATTCACTGCAGATACATCCTCCAACACAGCCAAC
ATGCAACTCAGCAGCCTGACATCTGAGGACTCTGCCGTCTATTACTGTGCAAGATTTGAT
GGTAACTACGGTTACTATGCTATGGACTACTGGGGTCAAGGATCCTCAGTCACCGTCTCC
TCA
SEQ ID NO: 14 52M51 Heavy chain variable region ammo acid sequence
without putative signal sequence
QVQLQQSGAELMKPGASVKISCKAAGYTMRGYWIEWIKQRPGHGLEWIGQILPGTGRTNY
NEKFKGKATFTADTSSNTANMQLSSLTSEDSAVYYCARFDGNYGYYAMDYWGQGSSVTVS
SA
SEQ ID NO: 15 52M51 Heavy chain CDR1
RGYWIE
SEQ ID NO: 16 52M51 Heavy chain CDR2
QILPGTGRTNYNEKEKG
SEQ ID NO: 17 52M51 Heavy chain CDR3
FDGNYGYYAMDY
SEQ ID NO: 18 52M51 Light chain CDR1
RSSTGAVTTSNYAN
SEQ ID NO: 19 52M51 Light chain CDR2
GTNNRAP
SEQ ID NO: 20 52M51 Light chain CDR3
ALWYSNEIWVEGGGTKL
Humanized 52M51 sgswems
SEQ ID NO: 21 52M51-H4 Heavy chain polynucleotide sequence (Putative
signal sequence underlined)
ATGGATTGGACATGGAGGGTGTTCTGCCTCCTCGCTGTGGCTCCTGGAGTCCTGAGCCAG
GTCCAGCTCGTCCAGAGCGGGGCTGAAGTCAAGAAGCCTGGCGCTAGCGTCAAAATCAGC
TGTAAGGTCAGCGGATACACACTGAGGGGATACTGGATCGAGTGGGTGAGGCAGGCTCCA
GGAAAGGGCCTGGAATGGATCGGCCAGATCCTGCCTGGAACCGGAAGGACAAATTACAAT
GAGAAGTTTAAGGGAAGGGTCACAATGACAGCAGACACAAGCACAGACACAGCTTATATG
GAACTCAGCTCCCTCAGATCCGAGGACACCGCTGTCTACTATTGTGCCAGGTTCGATGGA
AATTACGGATACTATGCCATGGATTACTGGGGACAGGGGACAACGGTCACCGTGAGCTCA
GCCAGCACAAAGGGCCCTAGCGTCTTCCCTCTGGCTCCCTGCAGCAGGAGCACCAGCGAG
AGCACAGCCGCCCTGGGCTGCCTGGTCAAGGACTACTTCCCCGAACCGGTGACGGTGTCG
TGGAACTCAGGCGCTCTGACCAGCGGCGTGCACACCTTCCCAGCTGTCCTACAGTCCTCA
GGACTCTACTCCCTCAGCAGCGTGGTGACCGTGCCCTCCAGCAACTTCGGCACCCAGACC
TACACCTGCAACGTAGATCACAAGCCCAGCAACACCAAGGTGGACAAGACAGTTGAGCGC
AAATGTTGTGTCGAGTGCCCACCGTGCCCAGCACCACCTGTGGCAGGACCGTCAGTCTTC
CTCTTCCCCCCAAAACCCAAGGACACCCTCATGATCTCCCGGACCCCTGAGGTCACGTGC
GTGGTGGTGGACGTGAGCCACGAAGACCCCGAGGTCCAGTTCAACTGGTACGTGGACGGC
GTGGAGGTGCATAATGCCAAGACAAAGCCACGGGAGGAGCAGTTCAACAGCACGTTCCGT
GTGGTCAGCGTCCTCACCGTTGTGCACCAGGACTGGCTGAACGGCAAGGAGTACAAGTGC
AAGGTCTCCAACAAAGGCCTCCCAGCCCCCATCGAGAAAACCATCTCCAAAACCAAAGGG
CAGCCCCGAGAACCACAGGTGTACACCCTGCCCCCATCCCGGGAGGAGATGACCAAGAAC
CAGGTCAGCCTGACCTGCCTGGTCAAAGGCTTCTACCCCAGCGACATCGCCGTGGAGTGG
GAGAGCAATGGGCAGCCGGAGAACAACTACAAGACCACACCTCCCATGCTGGACTCCGAC
GGCTCCTTCTTCCTCTACAGCAAGCTCACCGTGGACAAGAGCAGGTGGCAGCAGGGGAAC
GTCTTCTCATGCTCCGTGATGCATGAGGCTCTGCACAACCACTACACGCAGAAGAGCCTC
TCCCTGTCTCCGGGTAAATGA
SEQ ID NO: 22 52M51-H4 Heavy chain amino acid sequence (Putative
signal sequence underlined)
MDQTWRVFCLLAVAPGVLSQVQLVQSGAEVKKPGASVKISCKVSGYTLRGYWIEWVRQAP
GKGLEWIGQILPGTGRTNYNEKFKGRVTMTADTSTDTAYMELSSLRSEDTAVYYCARFDG
NYGYYAMDYWGQGTTVTVSSASTKGPSVFPLAPCSRSTSESTAALGCLVKDYFPEPVTVS
WNSGALTSGVHTFPAVLQSSGLYSLSSVVTVPSSNFGTQTYTCNVDHKPSNTKVDKTVER
KCCVECPPCPAPPVAGPSVFLFPPKPKDTLMISRTPEVTCVVVDVSHEDPEVQFNWYVDG
VEVHNAKTKPREEQFNSTFRVVSVLTVVHQDWLNGKEYKCKVSNKGLPAPIEKTISKTKG
QPREPQVYTLPPSREEMTKNQVSLTCLVKGFYPSDIAVEWESNGQPENNYKTTPPMIDSD
GSFFLYSKLTVDKSRWQQGNVFSCSVMHEALHNYTQKSLSLSPGK
SEQ ID NO: 23 52M51-H4 Heavy chain variable region amino acid sequence
(Putative signal sequence underlined)
MDWTWRVFCLLAVAPGVLSQVQLVQSGAEVKKPGASVKISCKVSGYTLRGYWIEWVRQAP
GKGLEWIGQILPGTGRTNYEKFKGRVTMTADTSTDTAYMFELSSLRSEDTAVYYCARFDG
NYGYYAMDYWGQGTTVTVSSA
SEQ ID NO: 24 52M51-H4 Heavy chain variable region amino acid sequence
without putative signal sequence
QVQLVQSGAEVKKPGASVKISCKVSGYTLRGYWIEWVRQAPGKGLEWIGQILPGTGRTNY
NEKFKGRVTMTADTSTDTAYMELSSLRSEDTAVYYCARFDGNYGYYAMDYWGQGTTVTVS
SA
SEQ ID NO: 25 52M51-L3 Light chain polynucleotide sequence (Putative
signal sequence underlined)
ATGAGCGTCCCTACAATGGCTTGGATGATGCTCCTGCTGGGACTCCTGGCTTATGGAAGC
GGAGTGGATAGCCAGGCCGTCGTCACACAGGAACCTAGCCTCACCGTTAGCCCTGGAGGA
ACAGTCACACTGACCTGTAGGAGCTCCACAGGAGCTGTGACAACAAGCAATTACGCTAAC
TGGTTCCAGCAGAAGCCCGGTCAAGCCCCTAGAACCCTCATCGGCGGCACCAATAAGAGA
GCTCCCGGAGTCCCCGCCAGGTTCTCCGGCTCCCTCCTGGGTGGCAAGGCTGCTCTGACA
CTCAGCGGTGCCCAGCCAGAGGATGAAGCGGAGTACTACTGTGCACTGTGGTACAGCAAC
CATTGGGTTTTCGGAGGCGGAACAAAGTTAACCGTCCTCGGGCAGCCTAAGGCTGCTCCT
AGCGTCACACTGTTCCCCCCATCTAGCGAGGAGCTGCAGGCTAACAAGGCAACCCTCGTC
TGCCTGGTTAGCGACTTCTACCCTGGCGCTGTCACAGTGGCCTGGAAAGCTGACGGCTCC
CCTGTGAAAGTTGGCGTCGAAACCACAAAGCCTTCTAAGCAGAGCAATAATAAATATGCC
GCAAGCTCCTACCTCTCCCTGACTCCTGAGCAGTGGAAAAGCCATAGGAGCTACTCCTGC
CGGGTCACACACGAAGGAAGCACAGTGGAAAAGACAGTCGCCCCTGCTGAGTGTAGCTGA
SEQ ID NO: 26 52M51-L3 Light chain amino acid sequence (Putative
signal sequence underlined)
MSVPTMAWMMLLLGLLAYGSGVDSQAVVTQEPSLTVSPGGTVTLTCRSSTGAVTTSNYAN
WFQQKPGQAPRTLIGGTNNRAPGVPARFSGSLLGGKAALTLSGAQPEDEAEYYCALWYSN
HWVFGGGTKLTVLGQPKAAPSVTLFPPSSEELQANKATLVCLVSDFYPGAVTVAWKADGS
PVKVGVETTKPSKQSNNKYAASSYLSLTPEQWKSHRSYSCRVTHEGSTVEKTVAPAECS
SEQ ID NO: 27 52M51-L3 Light chain variable region amino acid sequence
(Putative signal sequence underlined)
MSVPTMAWMMLLLGLLAYGSGVDSQAVVTQEPSLTVSPGGTVTLTCRSSTGAVTTSNYAN
WFQQKPGQAPRTLIGGTNNRAPGVPARFSGSLLGGKAALTLSGAQPEDEAEYYCALWYSN
HWVFGGGTKLTVLG
SEQ ID NO: 28 52M51-L3 Light chain variable region amino acid sequence
without putative signal sequence
SGVDSQAVVTQEPSLTVSPGGTVTLTCRSSTGAVTTSNYANWFQQKPGQAPRTLIGGTNN
RAPGVPARFSGSLLGGKAALTLSGAQPEDEAEYYCALWYSNHWVFGGGTKLTVLG
SEQ ID NO: 29 52M51-L4 Light chain polynucleotide sequence (Putative
signal sequence underlined)
ATGAGCGTCCCTACAATGGCTTGGATGATGCTCCTGCTGGGACTCCTGGCTTATGGAAGC
GGAGTGGATAGCCAGACCGTCGTCACACAGGAACCTAGCTTTTCCGTTAGCCCTGGAGGA
ACAGTCACACTGACCTGTAGGAGCTCCACAGGAGCTGTGACAACAAGCAATTACGCTAAC
TGGTATCAGCAGACTCCCGGTCAAGCCCCTAGAACCCTCATCGGCGGCACCAATAACAGA
GCTCCCGGAGTCCCCGACAGGTTCTCCGGCTCCATCCTGGGAAATAAAGCTGCTCTGACA
ATCACAGGTGCCCAGGCTGACGATGAAAGCGACTACTACTGTGCACTGTGGTACAGCAAC
CATTGGGTTTTCGGAGGCGGAACAAAGTTAACCGTCCTCGGGCAGCCTAAGGCTGCTCCT
AGCGTCACACTGTTCCCCCCATCTAGCGAGGAGCTGCAGGCTAACAAGGCAACCCTCGTC
TGCCTGGTTAGCGACTTCTACCCTGGCGCTGTCACAGTGGCCTGGAAAGCTGACGGCTCC
CCTGTGAAAGTTGGCGTCGAAACCACAAAGCCTTCTAAGCAGAGCAATAATAAATATGCC
GCAAGCTCCTACCTCTCCCTGACTCCTGAGCAGTGGAAAAGCCATAGGAGCTACTCCTGC
CGGGTCACACACGAAGGAAGCACAGTGGAAAAGACAGTCGCCCCTGCTGAGTGTAGCTGA
SEQ ID NO: 30 52M51-L4 Light chain amino acid sequence (Putative
signal sequence underlined)
MSVPTMAWMMLLLGLLAYGSGVDSQTVVTQEPSFSVSPGGTVTLTCRSSTGAVTTSNYAN
WYQQTPGQAPRTLIGGTNNRAPGVPDRFSGSILGNKAALTITGAQADDESDYYCALWYSN
HWVFGGGTKLTVLGQPKAAPSVTLFPPSSEELQANKATLVCLVSDFYPGAVTVAWKADGS
PVKVGVETTKPSKQSNNKYAASSYLSLTPEQWKSHRSYSCRVTHEGSTVEKTVAPAECS
SEQ ID NO: 31 52M51-L4 Light chain variable region amino acid sequence
(Putative signal sequence underlined)
MSVPTMAWMMLLLGLLAYGSGVDSQTVVTQEPSFSVSPGGTVTLTCRSSTGAVTTSNYAN
WYQQTPGQAPRTLIGGTNNRAPGVPDRFSGSILGNKAALTITGAQADDESDYYCALWYSN
HWVFGGGTKLTVLG
SEQ ID NO: 32 52M51-L4 Light chain variable region amino acid sequence
without putative signal sequence
SGVDSQTVVTQEPSFSVSPGGTVTLTCRSSTGAVTTSNYANWYQQTPGQAPRTLIGGTNN
RAPGVPDRFSGSILGNKAALTITGAQADDESDYYCALWYSNHWVFGGGTKLTVLG
SEQ ID NO: 33 FLAG-tag
DYKDDDDK
SEQ ID NO: 34 Human NOTCH1 gene (wild-type)
ATGCCGCCGCTCCTGGCGCCCCTGCTCTGCCTGGCGCTGCTGCCCGCGCTCGCCGCACGA
GGCCCGCGATGCTCCCAGCCCGGTGAGACCTGCCTGAATGGCGGGAAGTGTGAAGCGGCC
AATGGCACGGAGGCCTGCGTCTGTGGCGGGGCCTTCGTGGGCCCGCGATGCCAGGACCCC
AACCCGTGCCTCAGCACCCCCTGCAAGAACGCCGGGACATGCCACGTGGTGGACCGCAGA
GGCGTGGCAGACTATGCCTGCAGCTGTGCCCTGGGCTTCTCTGGGCCCCTCTGCCTGACA
CCCCTGGACAATGCCTGCCTCACCAACCCCTGCCGCAACGGGGGCACCTGCGACCTGCTC
ACGCTGACGGAGTACAAGTGCCGCTGCCCGCCCGGCTGGTCAGGGAAATCGTGCCAGCAG
GCTGACCCGTGCGCCTCCAACCCCTGCGCCAACGGTGGCCAGTGCCTGCCCTTCGAGGCC
TCCTACATCTGCCACTGCCCACCCAGCTTCCATGGCCCCACCTGCCGGCAGGATGTCAAC
GAGTGTGGCCAGAAGCCCGGGCTTTGCCGCCACGGAGGCACCTGCCACAACGAGGTCGGC
TCCTACCGCTGCGTCTGCCGCGCCACCCACACTGGCCCCAACTGCGAGCGGCCCTACGTG
CCCTGCAGCCCCTCGCCCTGCCAGAACGGGGGCACCTGCCGCCCCACGGGCGACGTCACC
CACGAGTGTGCCTGCCTGCCAGGCTTCACCGGCCAGAACTGTGAGGAAAATATCGACGAT
TGTCCAGGAAACAACTGCAAGAACGGGGGTGCCTGTGTGGACGGCGTGAACACCTACAAC
TGCCGCTGCCCGCCAGAGTGGACAGGTCAGTACTGTACCGAGGATGTGGACGAGTGCCAG
CTGATGCCAAATGCCTGCCAGAACGGCGGGACCTGCCACAACACCCACGGTGGCTACAAC
TGCGTGTGTGTCAACGGCTGGACTGGTGAGGACTGCAGCGAGAACATTGATGACTGTGCC
AGCGCCGCCTGCTTCCACGGCGCCACCTGCCATGACCGTGTGGCCTCCTTCTACTGCGAG
TGTCCCCATGGCCGCACAGGTCTGCTGTGCCACCTCAACGACGCATGCATCAGCAACCCC
TGTAACGAGGGCTCCAACTGCGACACCAACCCTGTCAATGGCAAGGCCATCTGCACCTGC
CCCTCGGGGTACACGGGCCCGGCCTGCAGCCAGGACGTGGATGAGTGCTCGCTGGGTGCC
AACCCCTGCGAGCATGCGGGCAAGTGCATCAACACGCTGGGCTCCTTCGAGTGCCAGTGT
CTGCAGGGCTACACGGGCCCCCGATGCGAGATCGACGTCAACGAGTGCGTCTCGAACCCG
TGCCAGAACGACGCCACCTGCCTGGACCAGATTGGGGAGTTCCAGTGCATCTGCATGCCC
GGCTACGAGGGTGTGCACTGCGAGGTCAACACAGACGAGTGTGCCAGCAGCCCCTGCCTG
CACAATGGCCGCTGCCTGGACAAGATCAATGAGTTCCAGTGCGAGTGCCCCACGGGCTTC
ACTGGGCATCTGTGCCAGTACGATGTGGACGAGTGTGCCAGCACCCCCTGCAAGAATGGT
GCCAAGTGCCTGGACGGACCCAACACTTACACCTGTGTGTGCACGGAAGGGTACACGGGG
ACGCACTGCGAGGTGGACATCGATGAGTGCGACCCCGACCCCTGCCACTACGGCTCCTGC
AAGGACGGCGTCGCCACCTTCACCTGCCTCTGCCGCCCAGGCTACACGGGCCACCACTGC
GAGACCAACATCAACGAGTGCTCCAGCCAGCCCTGCCGCCACGGGGGCACCTGCCAGGAC
CGCGACAACGCCTACCTCTGCTTCTGCCTGAAGGGGACCACAGGACCCAACTGCGAGATC
AACCTGGATGACTGTGCCAGCAGCCCCTGCGACTCGGGCACCTGTCTGGACAAGATCGAT
GGCTACGAGTGTGCCTGTGAGCCGGGCTACACAGGGAGCATGTGTAACATCAACATCGAT
GAGTGTGCGGGCAACCCCTGCCACAACGGGGGCACCTGCGAGGACGGCATCAATGGCTTC
ACCTGCCGCTGCCCCGAGGGCTACCACGACCCCACCTGCCTGTCTGAGGTCAATGAGTGC
AACAGCAACCCCTGCGTCCACGGGGCCTGCCGGGACACCCTCAACGGGTACAAGTGCGAC
TGTGACCCTGGGTGGAGTGGGACCAACTGTGACATCAACAACAATGAGTGTGAATCCAAC
CCTTGTGTCAACGGCGGCACCTGCAAAGACATGACCAGTGGCTACGTGTGCACCTGCCGG
GAGGGCTTCAGCGGTCCCAACTGCCAGACCAACATCAACGAGTGTGCGTCCAACCCATGT
CTGAACCAGGGCACGTGTATTGACGACGTTGCCGGGTACAAGTGCAACTGCCTGCTGCCC
TACACAGGTGCCACGTGTGAGGTGGTGCTGGCCCCGTGTGCCCCCAGCCCCTGCAGAAAC
GGCGGGGAGTGCAGGCAATCCGAGGACTATGAGAGCTTCTCCTGTGTCTGCCCCACGGGC
TGGCAAGGGCAGACCTGTGAGGTCGACATCAACGAGTGCGTTCTGAGCCCGTGCCGGCAC
GGCGCATCCTGCCAGAACACCCACGGCGGCTACCGCTGCCACTGCCAGGCCGGCTACAGT
GGGCGCAACTGCGAGACCGACATCGACGACTGCCGGCCCAACCCGTGTCACAACGGGGGC
TCCTGCACAGACGGCATCAACACGGCCTTCTGCGACTGCCTGCCCGGCTTCCGGGGCACT
TTCTGTGAGGAGGACATCAACGAGTGTGCCAGTGACCCCTGCCGCAACGGGGCCAACTGC
ACGGACTGCGTGGACAGCTACACGTGCACCTGCCCCGCAGGCTTCAGCGGGATCCACTGT
GAGAACAACACGCCTGACTGCACAGAGAGCTCCTGCTTCAACGGTGGCACCTGCGTGGAC
GGCATCAACTCGTTCACCTGCCTGTGTCCACCCGGCTTCACGGGCAGCTACTGCCAGCAC
GATGTCAATGAGTGCGACTCACAGCCCTGCCTGCATGGCGGCACCTGTCAGGACGGCTGC
GGCTCCTACAGGTGCACCTGCCCCCAGGGCTACACTGGCCCCAACTGCCAGAACCTTGTG
CACTGGTGTGACTCCTCGCCCTGCAAGAACGGCGGCAAATGCTGGCAGACCCACACCCAG
TACCGCTGCGAGTGCCCCAGCGGCTGGACCGGCCTTTACTGCGACGTGCCCAGCGTGTCC
TGTGAGGTGGCTGCGCAGCGACAAGGTGTTGACGTTGCCCGCCTGTGCCAGCATGGAGGG
CTCTGTGTGGACGCGGGCAACACGCACCACTGCCGCTGCCAGGCGGGCTACACAGGCAGC
TACTGTSAGGACCTGGTGGACGAGTGCTCACCCAGCCCCTGCCAGAACGGGGCCACCTGC
ACGGACTACCTGGGCGGCTACTCCTGCAAGTGCGTGGCCGGCTACCACGGGGTGAACTGC
TCTGAGGAGATCGACGAGTGCCTCTCCCACCCCTGCCAGAACGGGGGCACCTGCCTCGAC
CTCCCCAACACCTACAAGTGCTCCTGCCCACGGGGCACTCAGGGTGTGCACTGTGAGATC
AACGTGGACGACTGCAATCCCCCCGTTGACCCCGTGTCCCGGAGCCCCAAGTGCTTTAAC
AACGGCACCTGCGTGGACCAGGTGGGCGGCTACAGCTGCACCTGCCCGCCGGGCTTCGTG
GGTGAGCGCTGTGAGGGGGATGTCAACGAGTGCCTGTCCAATCCCTGCGACGCCCGTGGC
AGCCAGAACTGCGTGCAGCGCGTCAATGACTTCCACTGCGAGTGCCGTGCTGGTCACACC
GGGCGCCGCTGCGAGTCCGTCATCAATGGCTGCAAAGGCAAGCCCTGCAAGAATGGGGGC
ACCTGCGCCGTGGCCTCCAACACCGCCCGCGGGTTCATCTGCAAGTGCCCTGCGGGCTTC
GAGGGCGCCACGTGTGAGAATGACGCTCGTACCTGCGGCAGCCTGCGCTGCCTCAACGGC
GGCACATGCATCTCCGGCCCGCGCAGCCCCACCTGCCTGTGCCTGGGCCCCTTCACGGGC
CCCGAATGCCAGTTCCCGGCCAGCAGCCCCTGCCTGGGCGGCAACCCCTGCTACAACCAG
GGGACCTGTGAGCCCACATCCGAGAGCCCCTTCTACCGTTGCCTGTGCCCCGCCAAATTC
AACGGGCTCTTGTGCCACATCCTGGACTACAGCTTCGGGGGTGGGGCCGGGCGCGACATC
CCCCCGCCGCTGATCGAGGAGGCGTGCGAGCTGCCCGAGTGCCAGGAGGACGCGGGCAAC
AAGGTCTGCAGCCTGCAGTGCAACAACCACGCGTGCGGCTGGGACGGCGGTGACTGCTCC
CTCAACTTCAATGACCCCTGGAAGAACTGCACGCAGTCTCTGCAGTGCTGGAAGTACTTC
AGTGACGGCCACTGTGACAGCCAGTGCAACTCAGCCGGCTGCCTCTTCGACGGCTTTGAC
TGCCAGCGTGCGGAAGGCCAGTGCAACCCCCTGTACGACCAGTACTGCAAGGACCACTTC
AGCGACGGGCACTGCGACCAGGGCTGCAACAGCGCGGAGTGCGAGTGGGACGGGCTGGAC
TGTGCGGAGCATGTACCCGAGAGGCTGGCGGCCGGCACGCTGGTGGTGGTGGTGCTGATG
CCGCCGGAGCAGCTGCGCAACAGCTCCTTCCACTTCCTGCGGGAGCTCAGCCGCGTGCTG
CACACCAACGTGGTCTTCAAGCGTGACGCACACGGCCAGCAGATGATCTTCCCCTACTAC
GGCCGCGAGGAGGAGCTGCGCAAGCACCCCATCAAGCGTGCCGCCGAGGGCTGGGCCGCA
CCTGACGCCCTGCTGGGCCAGGTGAAGGCCTCGCTGCTCCCTGGTGGCAGCGAGGGTGGG
CGGCGGCGGAGGGAGCTGGACCCCATGGACGTCCGCGGCTCCATCGTCTACCTGGAGATT
GACAACCGGCAGTGTGTGCAGGCCTCCTCGCAGTGCTTCCAGAGTGCCACCGACGTGGCC
GCATTCCTGGGAGCGCTCGCCTCGCTGGGCAGCCTCAACATCCCCTACAAGATCGAGGCC
GTGCAGAGTGAGACCGTGGAGCCGCCCCCGCCGGCGCAGCTGCACTTCATGTACGTGGCG
GCGGCCGCCTTTGTGCTTCTGTTCTTCGTGGGCTGCCGGGTGCTGCTGTCCCGCAAGCGC
CGGCGGCAGCATGGCCAGCTCTGGTTCCCTGAGGGCTTCAAAGTGTCTGAGGCCAGCAAG
AAGAAGCGGCGGGAGCCCCTCGGCGAGGACTCCGTGGGCCTCAAGCCCCTGAAGAACGCT
TCAGACGGTGCCCTCATGGACGACAACCAGAATGAGTGGGGGGACGAGGACCTGGAGACC
AAGAAGTTCCGGTTCGAGGAGCCCGTGGTTCTGCCTGACCTGGACGACCAGACAGACCAC
CGGCAGTGGACTCAGCAGCACCTGGATGCCGCTGACCTGCGCATGTCTGCCATGGCCCCC
ACACCGCCCCAGGGTGAGGTTGACGCCGACTGCATGGACGTCAATGTCCGCGGGCCTGAT
GGCTTCACCCCGCTCATGATCGCCTCCTGCAGCGGGGGCGGCCTGGAGACGGGCAACAGC
GAGGAAGAGGAGGACGCGCCGGCCGTCATCTCCGACTTCATCTACCAGGGCGCCAGCCTG
CACAACCAGACAGACCGCACGGGCGAGACCGCCTTGCACCTGGCCGCCCGCTACTCACGC
TCTGATGCCGCCAAGCGCCTGCTGGAGGCCAGCGCAGATGCCAACATCCAGGACAACATG
GGCCGCACCCCGCTGCATGCGGCTGTGTCTGCCGACGCACAAGGTGTCTTCCAGATCCTG
ATCCGGAACCGAGCCACAGACCTGGATGCCCGCATGCATGATGGCACGACGCCACTGATC
CTGGCTGCCCGCCTGGCCGTGGAGGGCATGCTGGAGGACCTCATCAACTCACACGCCGAC
GTCAACGCCGTAGATGACCTGGGCAAGTCCGCCCTGCACTGGGCCGCCGCCGTGAACAAT
GTGGATGCCGCAGTTGTGCTCCTGAAGAACGGGGCTAACAAAGATATGCAGAACAACAGG
GAGGAGACACCCCTGTTTCTGGCCGCCCGGGAGGGCAGCTACGAGACCGCCAAGGTGCTG
CTGGACCACTTTGCCAACCGGGACATCACGGATCATATGGACGGCCTGCCGCGCGACATC
GCACAGGAGCGCATGCATCACGACATCGTGAGGCTGCTGGACGAGTACAACCTGGTGCGC
AGCCCGCAGCTGCACGGAGCCCCGCTGGGGGGCACGCCCACCCTGTCGCCCCCGCTCTGC
TCGCCCAACGGCTACCTGGGCAGCCTCAAGCCCGGCGTGCAG3GCAAGAAGGTCCGCAAG
CCCAGCAGCAAAGGCCTGGCCTGTGGAAGCAAGGAGGCCAAGGACCTCAAGGCACGGAGG
AAGAAGTCCCAGGACGGCAAGGGCTGCCTGCTGGACAGCTCCGGCATGCTCTCGCCCGTG
GACTCCCTGGAGTCACCCCATGGCTACCTGTCAGACGTGGCCTCGCCGCCACTGCTGCCC
TCCCCGTTCCAGCAGTCTCCGTCCGTGCCCCTCAACCACCTGCCTGGGATGCCCGACACC
CACCTGGGCATCGGGCACCTGAACGTGGCGGCCAAGCCCGAGATGGCGGCGCTGGGTGGG
GGCGGCCGGCTGGCCTTTGAGACTGGCCCACCTCGTCTCTCCCACCTGCCTGTGGCCTCT
GGCACCAGCACCGTCCTGGGCTCCAGCAGCGGAGGGGCCCTGAATTTCACTGTGGGCGGG
TCCACCAGTTTGAATGGTCAATGCGAGTGGCTGTCCCGGCTGCAGAGCGGCATGGTGCCG
AACCAATACAACCCTCTGCGGGGGAGTGTGGCACCAGGCCCCCTGAGCACACAGGCCCCC
TCCCTGCAGCATGGCATGGTAGGCCCGCTGCACAGTAGCCTTGCTGCCAGCGCCCTGTCC
CAGATGATGAGCTACCAGGGCCTGCCCAGCACCCGGCTGGCCACCCAGCCTCACCTGGTG
CAGACCCAGCAGGTGCAGCCACAAAACTTACAGATGCAGCAGCAGAACCTGCAGCCAGCA
AACATCCAGCAGCAGCAAAGCCTGCASCCGCCACCACCACCACCACAGCCGCACCTTGGC
GTGAGCTCAGCAGCCAGCGGCCACCTGGGCCGGAGCTTCCTGAGTGGAGAGCCGAGGCAG
GCAGACGTGCAGCCACTGGGCCCCAGCAGCCTGGCGGTGCACACTATTCTGCCCCAGGAG
AGCCCCGCCCTGCCCACGTCGCTGCCATCCTCGCTGGTCCCACCCGTGACCGCAGCCCAG
TTCCTGACGCCCCCCTCGCAGCACAGCTACTCCTCGCCTGTGGACAACACCCCCAGCCAC
CAGCTACAGGTGCCTGAGCACCCCTTCCTCACCCCGTCCCCTGAGTCCCCTGACCAGTGG
TCCAGCTCGTCCCCGCATTCCAACGTCTCCGACTGGTCCGAGGGCGTCTCCAGCCCTCCC
ACCAGCATGCAGTCCCAGATCGCCCGCATTCCGGAGGCCTTCAAGTAAACGGCGCGCCCC
ACGAGACCCCGGCTTCCTTTCCCAAGCCTTCGGGCGTCTGTGTGCGCTCTGTGGATGCCA
GGGCCGACCAGAGGAGCCTTTTTAAAACACATGTTTTTATACAAAATAAGAACGAGGATT
TTAATTTTTTTTAGTATTTATTTATGTACTTTTATTTTACACAGAAACACTGCCTTTTTA
TTTATATGTACTGTTTTATCTGGCCCCAGGTAGAAACTTTTATCTATTCTGAGAAAACAA
GCAAGTTCTGAGAGCCAGGGTTTTCCTACGTAGGATGAAAAGATTCTTCTGTGTTTATAA
AATATAAACAAAGATTCATGATTTATAAATGCCATTTATTTATTGATTCCTTTTTTCAAA
ATCCAAAAAGAAATGATGTTGGAGAAGGGAAGTTGAACGAGCATAGTCCAAAAAGCTCCT
GGGGCGTCCAGGCCGCGCCCTTTCCCCGACGCCCACCCAACCCCAAGCCAGCCCGGCCGC
TCCACCAGCATCACCTGCCTGTTAGGAGAAGCTGCATCCAGAGGCAAACGGAGGCAAAGC
TGGCTCACCTTCCGCACGCGGATTAATTTGCATCTGAAATAGGAAACAAGTGAAAGCATA
TGGGTTAGATGTTGCCATGTGTTTTAGATGGTTTCTTGCAAGCATGCTTGTGAAAATGTG
TTCTCGGAGTGTGTATGCCAAGAGTGCACCCATGGTACCAATCATGAATCTTTGTTTCAG
GTTCAGTATTATGTAGTTGTTCGTTGGTTATACAAGTTCTTGGTCCCTCCAGAACCACCC
CGGCCCCCTGCCCGTTCTTGAAATGTAGGCATCATGCATGTCAAACATGAGATGTGTGGA
CTGTGGCACTTGCCTGGGTCACACACGGAGGCATCCTACCCTTTTCTGGGGAAAGACACT
GCCTGGGCTGACCCCGGTGGCGGCCCCAGCACCTCAGCCTGCACAGTGTCCCCCAGGTTC
CGAAGAAGATGCTCCAGCAACACAGCCTGGGCCCCAGCTCGCGGGACCCGACCCCCCGTG
GGCTCCCGTGTTTTGTAGGAGACTTGCCAGAGCCGGGCACATTGAGCTGTGCAACGCCGT
GGGCTGCGTCCTTTGGTCCTGTCCCCGCAGCCCTGGCAGGGGGCATGCGGTCGGGCAGGG
GCTGGAGGGAGGCGGGGGCTGCCCTTGGGCCACCCCTCCTAGTTTGGGAGGAGCAGATTT
TTGCAATACCAAGTATAGCCTATGGCAGAAAAAATGTCTGTAAATATGTTTTTAAAGGTG
GATTTTGTTTAAAAAATCTTAATGAATGAGTCTGTTGTGTGTCATGCCAGTGAGGGACGT
CAGACTTGGCTCAGCTCGGGGAGCCTTAGGCGCCCATGCACTGGGGACGCTCCGCTGCCG
TGCCGCCTGCACTCCTCAGGGCAGCCTCCCCCGGCTCTACGGGGGCCGCGTGGTGCCATC
CCCAGGGGGCATGACCAGATGCGTCCCAAGATGTTGATTTTTACTGTGTTTTATAAAATA
GAGTGTAGTTTACAGAAAAAGACTTTAAAAGTGATCTACATGAGGAACTGTAGATGATGT
ATTTTTTTCATCTTTTTTGTTAACTGATTTGCAATAAAAATGATACTGATGGTGAAAAAA
AAAAAAAAA

What is claimed is:

1. A method of treating a hematologic cancer in a human subject comprising administering to the subject a therapeutically effective amount of an antibody that binds human NOTCH1, wherein the antibody comprises:
(a) a heavy chain CDR1 comprising RGYWIE (SEQ ID NO: 15), a heavy chain CDR2 comprising QILPGTGRTNYNEKFKG (SEQ ID NO:16), and a heavy chain CDR3 comprising FDGNYGYYAMDY (SEQ ID NO: 17); and/or
(b) a light chain CDR1 comprising RSSTGAVTTSNYAN (SEQ ID NO: 18), a light chain CDR2 comprising GTNNRAP (SEQ ID NO: 19), and a light chain CDR3 comprising ALWYSNHWVFGGGTKL (SEQ ID NO:20),
wherein the hematologic cancer is chronic lymphocytic leukemia (CLL), hairy cell leukemia, chronic myelogenous leukemia (CML), non-Hodgkin lymphoma, diffuse large B-cell lymphoma (DLBCL), mantle cell lymphoma (MCL), or cutaneous T-cell lymphoma (CTCL).

2. A method of treating a hematologic cancer in a human subject comprising administering to the subject a therapeutically effective amount of an antibody that binds human NOTCH1, wherein the antibody comprises:
(a) a heavy chain CDR1 comprising RGYWIE (SEQ ID NO: 15), or a variant thereof comprising 1, 2, 3, or 4 amino acid substitutions;
(b) a heavy chain CDR2 comprising QILPGTGRTNYNEKFKG (SEQ ID NO:16), or a variant thereof comprising 1, 2, 3, or 4 amino acid substitutions;
(c) a heavy chain CDR3 comprising FDGNYGYYAMDY (SEQ ID NO: 17), or a variant thereof comprising 1, 2, 3, or 4 amino acid substitutions;
(d) a light chain CDR1 comprising RSSTGAVTTSNYAN (SEQ ID NO: 18), or a variant thereof comprising 1, 2, 3, or 4 amino acid substitutions;
(e) a light chain CDR2 comprising GTNNRAP (SEQ ID NO: 19), or a variant thereof comprising 1, 2, 3, or 4 amino acid substitutions; and
(f) a light chain CDR3 comprising ALWYSNHWVFGGGTKL (SEQ ID NO:20), or a variant thereof comprising 1, 2, 3, or 4 amino acid substitutions,
wherein the hematologic cancer is chronic lymphocytic leukemia (CLL), hairy cell leukemia, chronic myelogenous leukemia (CML), non-Hodgkin lymphoma, diffuse large B-cell lymphoma (DLBCL), mantle cell lymphoma (MCL), or cutaneous T-cell lymphoma (CTCL).

3. The method of claim 2, wherein the amino acid substitutions are conservative amino acid substitutions.

4. A method of treating a hematologic cancer in a human subject comprising administering to the subject a therapeutically effective amount of an antibody that binds human NOTCH1, wherein the antibody comprises:
(a) a heavy chain variable region having at least about 90% sequence identity to SEQ ID NO:14 or SEQ ID NO:24; and/or
(b) a light chain variable region having at least about 90% sequence identity to SEQ ID NO:8, SEQ ID NO:28, or SEQ ID NO:32,
wherein the hematologic cancer is chronic lymphocytic leukemia (CLL), hairy cell leukemia, chronic myelogenous leukemia (CML), non-Hodgkin lymphoma, diffuse large B-cell lymphoma (DLBCL), mantle cell lymphoma (MCL), or cutaneous T-cell lymphoma (CTCL).

5. The method of claim 4, wherein the antibody comprises:
(a) a heavy chain variable region has at least about 95% sequence identity to SEQ ID NO: 14 or SEQ ID NO:24; and/or
(b) a light chain variable region has at least about 95% sequence identity to SEQ ID NO:8, SEQ ID NO:28, or SEQ ID NO:32.

6. The method of claim 4, wherein the antibody comprises:
(a) a heavy chain variable region comprising SEQ ID NO: 14 or SEQ ID NO:24; and/or
(b) a light chain variable region comprising SEQ ID NO:8, SEQ ID NO:28, or SEQ ID NO:32.

7. The method of claim 6, wherein the antibody comprises:
(a) a heavy chain variable region comprising SEQ ID NO: 14 and
(b) a light chain variable region comprising SEQ ID NO:8.

8. The method of claim 6, wherein the antibody comprises:
(a) a heavy chain variable region comprising SEQ ID NO:24 and
(b) a light chain variable region comprising SEQ ID NO:28.

9. The method of claim 6, wherein the antibody comprises:
(a) a heavy chain variable region comprising SEQ ID NO:24 and
(b) a light chain variable region comprising SEQ ID NO:32.

10. The method according to any one of claims 1-9, wherein the antibody is a monoclonal antibody.

11. The method according to any one of claims 1-9, wherein the antibody is a recombinant antibody, a chimeric antibody, a humanized antibody, a human antibody, or an antibody fragment.

12. The method according to any one of claims 1-11, wherein the antibody is a monospecific antibody or a bispecific antibody.

13. The method according to any one of claims 1-11, wherein the antibody is a monovalent antibody.

14. The method according to any one of claims 1-13, wherein the antibody is an IgA, IgD, IgE, IgG or IgM antibody.

15. The method of claim 14, wherein the antibody is an IgG1 or IgG2 antibody.

16. A method of treating a hematologic cancer in a human subject comprising administering to the subject a therapeutically effective amount of an antibody that binds human NOTCH1, wherein the antibody is a humanized version of the antibody produced by the hybridoma cell line deposited with the ATCC as 52M51 or Patent Deposit PTA-9405,
wherein the hematologic cancer is chronic lymphocytic leukemia (CLL), hairy cell leukemia, chronic myelogenous leukemia (CML), non-Hodgkin lymphoma, diffuse large B-cell lymphoma (DLBCL), mantle cell lymphoma (MCL), or cutaneous T-cell lymphoma (CTCL).

17. A method of treating a hematologic cancer in a human subject comprising administering to the subject a therapeutically effective amount of an antibody that binds human NOTCH1, wherein the antibody is encoded by the polynucleotide deposited with the ATCC as 52M51-H4L3 or Patent Deposit PTA-9549,
wherein the hematologic cancer is chronic lymphocytic leukemia (CLL), hairy cell leukemia, chronic myelogenous leukemia (CML), non-Hodgkin lymphoma, diffuse large B-cell lymphoma (DLBCL), mantle cell lymphoma (MCL), or cutaneous T-cell lymphoma (CTCL).

18. A method of treating a hematologic cancer in a human subject comprising administering to the subject a therapeutically effective amount of an antibody that binds human NOTCH1, wherein the antibody binds an epitope on human NOTCH1 that overlaps with the epitope on human NOTCH1 to which antibody 52M51 produced by the hybridoma cell line deposited as ATCC Patent Deposit PTA-9405 binds,
wherein the hematologic cancer is chronic lymphocytic leukemia (CLL), hairy cell leukemia, chronic myelogenous leukemia (CML), non-Hodgkin lymphoma, diffuse large B-cell lymphoma (DLBCL), mantle cell lymphoma (MCL), or cutaneous T-cell lymphoma (CTCL).

19. A method of treating a hematologic cancer in a human subject comprising administering to the subject a therapeutically effective amount of an antibody that binds human NOTCH1, wherein the antibody competes with antibody 52M51 for binding to human NOTCH1,
wherein the hematologic cancer is chronic lymphocytic leukemia (CLL), hairy cell leukemia, chronic myelogenous leukemia (CML), non-Hodgkin lymphoma, diffuse large B-cell lymphoma (DLBCL), mantle cell lymphoma (MCL), or cutaneous T-cell lymphoma (CTCL).

20. A method of inhibiting growth of hematologic cancer cells, comprising contacting the cancer cells with an effective amount of an antibody that binds human NOTCH1, and wherein the antibody comprises:
(a) a heavy chain CDR1 comprising RGYWIE (SEQ ID NO: 15), a heavy chain CDR2 comprising QILPGTGRTNYNEKFKG (SEQ ID NO: 16), and a heavy chain CDR3 comprising FDGNYGYYAMDY (SEQ ID NO:17); and/or
(b) a light chain CDR1 comprising RSSTGAVTTSNYAN (SEQ ID NO: 18), a light chain CDR2 comprising GTNNRAP (SEQ ID NO: 19), and a light chain CDR3 comprising ALWYSNHWVFGGGTKL (SEQ ID NO:20),
wherein the hematologic cancer is chronic lymphocytic leukemia (CLL), hairy cell leukemia, chronic myelogenous leukemia (CML), non-Hodgkin lymphoma, diffuse large B-cell lymphoma (DLBCL), mantle cell lymphoma (MCL), or cutaneous T-cell lymphoma (CTCL).

21. The method according to any one of claims 1-20, wherein the antibody binds the non-ligand binding membrane proximal region of human NOTCH1.

22. The method according to claim 21, wherein the antibody binds the non-ligand binding membrane proximal region of NOTCH1 comprises SEQ ID NO:2.

23. The method according to any one of claims 1-22, wherein the antibody is an antagonist of NOTCH1.

24. The method according to any one of claims 1-23, wherein the antibody inhibits activation of NOTCH1.

25. The method according to any one of claims 1-24, wherein the antibody inhibits NOTCH1 activity.

26. The method of claim 25, wherein the antibody inhibits activity of a constitutively activated NOTCH1.

27. The method according to any one of claims 1-26, wherein the antibody inhibits NOTCH1 signaling or inhibits release of the intracellular domain (ICD) of NOTCH1.

28. The method according to any one of claims 1-27, wherein the antibody inhibits cleavage within the membrane proximal region.

29. The method of claim 28, wherein the antibody inhibits cleavage at the S2 site within the membrane proximal region.

30. The method according to any one of claims 1-29, wherein the hematologic cancer is a cutaneous T-cell lymphoma (CTCL).

31. The method according to claim 30, wherein the hematologic cancer is Sézary Syndrome.

32. The method according to any one of claims 1-29, wherein the hematologic cancer is CLL.

33. The method according to any one of claims 1-29, wherein the hematologic cancer is MCL.

34. The method according to any one of claims 1-29, wherein the hematologic cancer is DLBCL.

35. The method according to any one of claims 1-29, wherein the subject has developed or is developing Richter's syndrome.

36. The method according to any one of claims 1-35, wherein the hematologic cancer comprises a NOTCH1 mutation.

37. The method of claim 36, wherein the NOTCH1 mutation is an activating mutation.

38. The method according to any one of claims 1-37, wherein the antibody inhibits growth of the cancer cells.

39. The method according to any one of the claims 1-38, which further comprises administering at least one additional therapeutic agent or therapy.

40. The method of claim 39, wherein the additional therapeutic agent is a chemotherapeutic agent.

41. The method of claim 39, wherein the additional therapeutic agent is an additional antibody therapeutic.

42. The method of claim 41, wherein the additional antibody therapeutic comprises an antibody that binds a NOTCH receptor other than NOTCH1.

43. The method of claim 41, wherein the additional antibody therapeutic comprises an antibody that binds a NOTCH receptor ligand.

44. The method according to any one of claims 1-43, wherein the antibody is conjugated to a cytotoxic agent.

45. The method according to any one of claims 1-44, wherein the antibody is administered after radiation therapy.

46. The method according to any one of claims 1-44, wherein the antibody is administered with radiation therapy.

47. The method of any one of claims 1-46, which further comprises a first step of determining that the subject's hematologic cancer comprises an activating mutation in the NOTCH1 gene prior to administration of the antibody.

48. The method of any one of claims 1-47, which further comprises selecting the subject for initial or additional treatment with the antibody based at least in part on the subject's hematologic cancer comprising a mutation in the NOTCH1 gene.

49. A method of selecting a human subject having a hematologic cancer for treatment with an antibody that binds to human NOTCH1, comprising determining whether a cancer cell from the subject has an activating mutation in the NOTCH1 gene, wherein if the cell has said mutation, the subject is selected for treatment with the antibody,
wherein the antibody is selected from the group consisting of
(a) an antibody comprising (i) a heavy chain CDR1 comprising RGYWIE (SEQ ID NO: 15), a heavy chain CDR2 comprising QILPGTGRTNYNEKFKG (SEQ ID NO: 16), and a heavy chain CDR3 comprising FDGNYGYYAMDY (SEQ ID NO:17); and (ii) a light chain CDR1 comprising RSSTGAVTTSNYAN (SEQ ID NO: 18), a light chain CDR2 comprising GTNNRAP (SEQ ID NO:19), and a light chain CDR3 comprising ALWYSNHWVFGGGTKL (SEQ ID NO:20);
(b) an antibody comprising (i) a heavy chain variable region comprising SEQ ID NO: 14 or SEQ ID NO:24; and (ii) a light chain variable region comprising SEQ ID NO:8, SEQ ID NO:28, or SEQ ID NO:32;
(c) an antibody produced by the hybridoma cell line deposited with the ATCC as 52M51 or Patent Deposit PTA-9405;
(d) an antibody encoded by the polynucleotide deposited with the ATCC as 52M51-H4L3 or Patent Deposit PTA-9549;
(e) an antibody that binds an epitope on human NOTCH1 that overlaps with the epitope on human NOTCH1 that any one of antibodies (a)-(e) binds; and
(f) an antibody that competes for binding to human NOTCH1 with any one of the antibodies (a)-(e);
and wherein the hematologic cancer is selected from the group consisting of chronic lymphocytic leukemia (CLL), hairy cell leukemia, chronic myelogenous leukemia (CML), non-Hodgkin lymphoma, diffuse large B-cell lymphoma (DLBCL), mantle cell lymphoma (MCL), and cutaneous T-cell lymphoma (CTCL).

50. The method of any one of claims 47-49, which further comprises a step of obtaining a body sample containing the cancer cells from the subject.

51. The method of claim 50, wherein the body sample is whole blood, plasma, serum, or tissue.

52. The method of any one of claims 47-51, wherein the step of determining whether cancer cells from the subject contain the mutation comprises nucleic acid sequencing.

53. An isolated polynucleotide encoding a mutant human NOTCH1 receptor, wherein said polynucleotide comprises a deletion at position 7444 or a missense mutation at position 4168 of the human NOTCH1 gene.

54. The isolated polynucleotide of claim 53, wherein the missense mutation is C4168A.

55. An isolated polypeptide encoded by the polynucleotide of claim 53 or 54.

56. A recombinant vector comprising the polynucleotide of claim 55.

57. A cell comprising the vector of claim 56.

58. A method of selecting a subject having diffuse large B-cell lymphoma (DLBCL) for treatment with an antibody that binds to human NOTCH1 and inhibits NOTCH1 activation or signaling comprising (a) determining whether cancer cells from the subject comprise an activating mutation in the NOTCH1 gene, and (b) selecting the subject whose cancer cells comprise the mutation for treatment with the antibody.

59. A method of treating a subject having DLBCL that comprises an activating mutation in the NOTCH1 gene, comprising administering to the subject a therapeutically effective amount of an antibody that binds to human NOTCH1 and inhibits activation or signaling of NOTCH1.

60. A method of treating a subject having DLBCL, comprising (a) determining that cancer cells from the subject comprise an activating mutation in the NOTCH1 gene, and (b) administering to the subject a therapeutically effective amount of an antibody that binds to human NOTCH1 and inhibits activation or signaling of NOTCH1.

61. The method of claim 58 or 60, which further comprises a step of obtaining a body sample containing the cancer cells from the subject.

62. The method of claim 61, wherein the body sample is whole blood, plasma, serum, or tissue.

63. The method of any one of claims 58 and 60-62, wherein the step of determining whether cancer cells from the subject comprise the mutation comprises nucleic acid sequencing.

64. The method of any one of claims 58-63, wherein the activating mutation is in the sequence of NOTCH1 encoding the PEST domain.

65. The method of any one of claims 58-64, wherein the activating mutation results in a truncation of the encoded NOTCH1 protein in the PEST domain.

66. The method of claim 65, wherein the mutation is a deletion at position 7444.

67. The method of any of claims 58-63, wherein the activating mutation is a missense mutation in the sequence of NOTCH1 encoding EGF domain 36.

68. The method of claim 67, wherein the activating mutation is at position 4168.

69. The method of claim 68, wherein the activating mutation is C4168A.

70. The method of any of claims 1-29, 34, 36-52 wherein the subject has DLBCL comprising an activating NOTCH1 mutation.

71. The method of claim 70, wherein the activating mutation is a mutation in the human NOTCH1 gene that results in a truncation of the encoded NOTCH1 protein in the PEST domain.

72. The method of claim 71, wherein the activating mutation is a deletion at position 7444.

73. The method of claim 70, wherein the activating mutation is a missense mutation in EGF domain 36.

74. The method of claim 73, wherein the activating mutation is a missense mutation at position 4168 of the human NOTCH1 gene.

75. The method of any one of claims 1-30 or 36-52, wherein the hematologic cancer is mycosis fungoides.

76. The method of claim 75, wherein the hematologic cancer is transformed mycosis fungoides.

77. The method of any one of claims 1-52 or 58-76, where the subject has previously failed a cancer therapy.
